(19) United States
(12) Patent Application Publication
(10) Pub. No.: US 2021/0205471 A1
(43) Pub. Date: Jul. 8, 2021

(54) CONCEPTS FOR THE TREATMENT OF GENETIC DISORDERS WITH HIGH-CAPACITY PLAL-GENERATED GOLD NANOPARTICLES

(71) Applicant: Zentrum für Forschungsförderung in der Pädiatrie GmbH, Hannover (DE)

(72) Inventors: Helmut Hanenberg, Düsseldorf (DE); Maj-Kristin Holz, Essen (DE); Katharina Jansen, Dortmund (DE); Katharina Waack-Buchholz, Essen (DE)

(21) Appl. No.: 17/155,411

(22) Filed: Jan. 22, 2021

Related U.S. Application Data
(63) Continuation of application No. PCT/EP2018/079997, filed on Nov. 2, 2018.
Application No. PCT/EP2018/079997 is a continuation of application No. PCT/EP2018/070453, filed on Jul. 27, 2018.

(51) Int. Cl. A61K 47/69; A61K 38/21; A61K 47/59; A61K 9/51; A61K 38/48; A61P 1/16; A61K 38/37

(52) U.S. Cl. CPC A61K 47/6929; A61K 38/215; A61K 47/59; A61K 38/37; A61K 38/4846; C12Y 304/21022; A61P 1/16; A61K 9/5192

(57) ABSTRACT

The present invention relates to conjugated gold nanoparticles, preferably for the use in the treatment of a monogenetic disorder resulting from a mutation in a gene coding for a liver-specific and/or liver-expressed protein, comprising laser-ablated gold nanoparticles, polyethylenimine (PEI) and/or derivatives and/or salts thereof and a nucleic acid molecule. Furthermore, the present invention relates to the use of such gold nanoparticles, a method for the preparation of conjugated gold nanoparticles, a nanoparticle-based delivery system and the use of such delivery system. In addition, the present invention relates to a method for transfection of target cells, a transfected target cell as well as a vector for the expression of a liver-specific and/or liver-expressed protein.

The present invention relates to the medical field of monogenetic disorders, in particular monogenetic disorders associated with mutations in genes coding for proteins expressed for example in the liver.

In particular, the present invention relates to conjugated gold nanoparticles, preferably for the use in the treatment of a monogenetic disorder resulting from a mutation in a gene coding for a liver-specific and/or liver-expressed protein, and the respective use of the particles.

Furthermore, the present invention relates a method for the preparation of conjugated gold nanoparticles, a nanoparticle-based delivery system and the use of the respective delivery system. A further subject of the present invention relates to a method for transfection of target cells and transfected target cells as such. Finally, the present invention relates to a vector to be used in the gold nanoparticles according to the present invention.

The liver is a vital organ of the human body and has a wide range of functions, including the detoxification of various metabolites, protein synthesis and the production of biochemicals necessary for digestion. Furthermore, the liver plays a central role in metabolism, regulation of glycogen storage, decomposition of red blood cells and hormone production.

As outlined before, one main function of the liver is the production of proteins and their subsequent secretion into the blood. Proteins produced and secreted by the liver include major plasma proteins, carrier proteins, hormones, prohormones and apolipoproteins. In particular, the liver produces and secretes proteins and factors, which regulate hemostasis, i.e. blood clotting.

Furthermore, the liver produces and secretes proteins involved in lipometabolism, amino acid metabolism, bilirubin metabolism, urea cycle metabolism, carbohydrate metabolism, proteoglycan metabolism and sphingolipid metabolism. Additionally, the liver produces the antiprotease alpha-1-antitrypsin as well as proteins involved in transportation processes.

Hemostasis occurs when blood is present outside of the body or blood vessels. During hemostasis three steps occur in a rapid sequence. The first step includes a vascular spasm or a vasoconstriction, respectively. By vasoconstriction, the amount of blood flow can be reduced and the blood loss can be limited. Furthermore, collagen is exposed at the site of injury, thereby promoting platelets to adhere to the injury site. The second step of hemostasis includes the formation of platelet plugs.

Thereby, platelets adhere to the damaged endothelium to form a plug. This process is also called primary hemostasis. Once the plug has been formed, clotting factors begin creating the clot. Thereby, the clotting factors begin to form fibrin factor (FIa). Fibrin is a fibrous, non-globular protein, which is formed by the action of the protease thrombin factor (FII). This third step of hemostasis including the coagulation is also called secondary hemostasis. Thereby, the platelet plug is reinforced, wherein fibrin threads function as glue for the sticky platelets.

A multitude of factors and proteins is involved in the secondary hemostasis, for example fibrinogen (FI), prothrombin (FII), tissue factor/tissue thromboplastin (FIII), calcium (FIV), proaccelerin (FV), proconvertin (FVII), antihemophilic factor A (FVIII), antihemophilic factor B (FIX), Stuart-Prower factor (FX), plasma thromboplastin antecedent (FXI), Hageman factor (FXII) and fibrin-stabilizing factor (FXIII), wherein the list of factors is not exhaustive with respect to factors and proteins regulating hemostasis.

A diminished or absent production of blood clotting factors can lead to a phenotype or disease called hemophilia. Hemophilia is a term for a group of blood clotting disorders whose clinical symptoms are caused by a diminished or absent activity of blood clotting factors. Hemophilia is a mostly inherited in particular monogenetic disorder that impairs the body's ability to make blood clots, a process needed to stop bleeding. People suffering from hemophilia usually bleed longer after an injury and bruise easily. Furthermore, the disorder leads to an increased risk of bleeding inside joints or the brain.

The two most common subforms are hemophilia A with an incidence of 1:10.000 due to loss-of-function mutations in the gene coding for coagulation factor FVIII and hemophilia B with an incidence of 1:50.000 due to mutations in the factor FIX gene. Hemophilia A and B are caused by inherited and also de novo mutations in the X-chromosomally localized FVIII and FIX genes, which lead to loss of protein activity and thereby interfere with the coagulation cascade causing severe bleeding episodes. Because of the X-chromosomal recessive inheritance, almost exclusively boys and men are affected, while females as heterozygous germ-line mutation carriers show a reduction of the factor activity measurable in the laboratory, but are clinically healthy, i.e. without symptoms. Based on the residual activity of FVIII or FIX in the plasma, severe (less than 1% activity), moderate (1 to 5% activity), mild (6 to 24% activity) and subhemophilia (25 to 50% activity) can be distinguished. Notably, more than 50% of patients are affected by severe hemophilia. Patients with severe and also moderate hemophilia suffer about 30 to 40 severe bleeding episodes per year. Bleeding occurs spontaneously or after slight trauma. Mild and subhemophilia are clinically apparent only after surgery, trauma or treatment with acetylsalicylic acid or related drugs.

The WHO currently estimates that the number of patients worldwide is >400.000, of which approximately 10.000 hemophils are living in Germany. The current therapy for clinically severe moderate hemophilia involves a regular prophylactic use of concentrated FVIII or FIX products by intravenous injections. This prophylaxis allows an almost normal life expectancy and quality of life for hemophilia patients. According to the scientific publication of Oldenburg: “Optimal treatment strategies for hemophilia: achievements and limitations of current prophylactic regiments”, published in Blood, 2015, 125(13):2038-44, in context with prophylactic treatment of hemophilia, concentrated FVIII or FIX products are either isolated as plasmatic factors from healthy blood donors or recovered as recombinant factors from specific cell cultures. A regular prophylaxis prevents long-lasting clinical consequences of the bleeding episodes including disabilities due to intracranial hemorrhage and chronic joint diseases and musculoskeletal crippling problems. Disadvantageously, the prophylactic treatment generates very high costs per year for each patient to be treated. Furthermore, the recurring treatments are rather stressful for the patients. Moreover, according to Peyvandi et al.: “A randomized trial of factor VIII and neutralizing antibodies in hemophilia A”, published in N. Engl. J. Med., 2016, 374(21):2054-64, more than 50% of patients with severe hemophilia do not produce any endogenous FVIII or FIX. In this patients, administration of the exogenous proteins results in the development of neutralizing antibodies, so-called inhibitors, in up to 45% of the cases. These inhibitors neutralize the substituted factors and thereby render the factor replacement therapy ineffective. In patients with inhibitors, immune tolerance induction can be achieved by treatment with high doses of factors over a period of one to two years. However, this approach is only successful in 50 to 70% of patients. Additionally, the immune tolerance induction leads to a significant increase of costs per patient per year.

Since hemophilia is—in the majority of cases—a monogenetic disorder, multiple efforts to treat the disease with different gene therapy strategies have been pursued. The basic goal of all gene therapy approaches is the permanent introduction of an intact copy of the defective gene as complementary DNA (cDNA) into the nucleus of the target cell.

Recombinant gene delivery systems for the intact gene are so-called vectors, which are mostly derived from viral systems. These wild-type viruses are evolutionarily optimized in terms of their properties to efficiently transfer their genetic information to the target cell and into the nucleus of the cell, respectively.

The viral gene transfer system most frequently used for hemophilia originates from the adeno-associated virus (AAV), which exists in various different serotypes and can infect primary liver cells particularly well. The use of an AAV-based gene transfer system has been described by High and Anguela: “Adeno-associated viral vectors for the treatment of hemophilia”, published in Hum. Mol. Genet., 2016, 25(R1):R36-41. In addition, lentiviral vectors derived from the human immunodeficiency virus (HIV-1) have been used and can very efficiently integrate into the DNA of dividing and also non-dividing cells. In all these viral approaches, the integration of the vector DNA into the genome of the target cell appears to be the greatest risk. Here, the function or expression of a gene located in the vicinity of the insertion site can be altered or modified by the integration event and thus can lead to a malignant transformation of the cell.

Another viral approach on the basis of a gene therapy for hemophilia B with an AAV-FIX vector was described by Nathwani et al. in the scientific publications “Adenovirus-associated virus vector-mediated gene transfer in hemophilia B”, N. Engl. J. Med., 2011, 365(25):2357-65, and “Long-term safety and efficacy of factor IX gene therapy in hemophilia B”, N. Engl. J. Med., 2014, 371(21):1994-2004. An important side effect or a severe adverse event, respectively, of the therapy was an increase of the liver enzymes. The liver toxicity required an additional cortisone therapy. Furthermore, patients once treated with a specific AAV serotype will develop lifelong immunity to the specific AAV envelope protein and can never be treated with the same vector or serotype again.

Similarly, a concept for a gene therapy for hemophilia A on the basis of an AAV-FVII vector has been developed. According to the scientific publication of Nault et al. “Recurrent AAV2-related insertional mutagenesis in human hepatocellular carcinomas”, Nat. Genet., 2015, 47(10):1187-93, a therapy on the basis of wildtype AAV might be linked with the risk of developing hepatocellular carcinoma in humans.

Moreover, non-genetic approaches for the treatment of hemophilia consist in the use of antibodies. In this context, for the treatment of hemophilia A, a bispecific humanized recombinant antibody has been described by Muto et al.: “Anti-factor IXa/x bispecific antibody ACE910 prevents joint bleeds in a long-term primate model of acquired hemophilia A”, published in Blood, 2014, 124(20):3165-71 as well as Kitazawa et al.: “A bispecific antibody to factors IXa and X restores factor VIII hemostatic activity in hemophilia A model”, published in Nat. Med. 2012, 18(10):1570-4. The respective antibody can replace the cross-linking of FIX or the active form FIXa, respectively, and FX as an essential function of FVIII in the coagulation cascade. Even though antibodies are not associated with the risk of mutagenesis, however, also a non-genetic therapy on the basis of antibodies can be linked with undesired side effects, in particular with respect to undesired immunological reactions.

Furthermore, with respect to gene therapy in general, efforts have been made with respect to the use of nanoparticles, such as chemically generated gold nanoparticles, as carrier for nucleic acids. In general, chemically generated gold nanoparticles are suitable to mediate a gene or DNA transfer to the target cells. Nevertheless, there use is linked with several adverse effects. On the one hand, in particular organic residues of the preparation process lead to a certain cell toxicity and undesired interactions between the particles, in particular agglomerations of particles. On the other hand, the loadability with transfection agents and genetic material is still not sufficient and linked with a reduced transfer of target DNA.

Overall, there is a strong need for improved therapeutic concepts and/or approaches with respect to the treatment of monogenetic diseases associated with mutations in genes coding for proteins predominantly expressed in the liver, in particular proteins of the coagulation cascade and/or proteins involved in hemostasis. Especially, there is a strong need for improved therapeutic concepts for the treatment of hemophilia.

Against the background of the severe disadvantages of known therapeutic concepts for the treatment of monogenetic disorders, in particular hemophilia, as delineated before, the problem of the present invention is based on the supply of a new therapeutic concept for the treatment of monogenetic disorders associated with mutations in genes coding for liver-specific and/or liver-expressed proteins and/or proteins predominantly expressed in the liver, in particular proteins involved in hemostasis and/or proteins or factors of the coagulation cascade.

In particular, the object of the present invention has to be seen in a therapeutic concept for the treatment of monogenetic disorders associated with the liver, especially hemophilia, on the basis of preferably non-viral gene therapy with an improved efficiency with respect to the transfer of genetic material as well as reduced side effects and a lowered cell toxicity.

The applicant has surprisingly found, that the aforementioned problem can be solved—according to the first aspect of the present invention—on the basis of a conjugated gold nanoparticles as claimed in claim 1; further, in particular advantageous embodiments of this aspect are subject-matter of the respective dependent claims.

Additionally, the present invention relates to—according to the second aspect of the present invention—the inventive use of the conjugated gold nanoparticles according to the respective independent claim; further, in particular advantageous embodiments of this aspect are subject-matter of the respective dependent claims.

Furthermore, subject-matter of the present invention is—according to the third aspect of the present invention—a method for the preparation of conjugated gold nanoparticles according to the respective independent claim; further, in particular advantageous embodiments of this aspect are subject-matter of the respective dependent claims.

The present invention also relates to—according to the fourth aspect of the present invention—a nanoparticle-based delivery system according to the respective independent claim; further, in particular advantageous embodiments of this aspect are subject-matter of the respective dependent claims.

In addition, subject-matter of the present invention is—according to the fifth aspect of the present invention—the use of a nanoparticle-based delivery system according to the respective independent claim; further, in particular advantageous embodiments of this aspect are subject-matter of the respective dependent claims.

Furthermore, the present invention relates to—according to a sixth aspect of the present invention—a method for transfection of target cells.

Another subject-matter of the present invention is—according to a seventh aspect of the present invention—a transfected target cell; further, in particular advantageous embodiments of this aspect are subject-matter of the respective dependent claims.

Finally, the present invention relates—according to an eighth aspect of the present invention—a vector for the expression of a liver-specific and/or liver expressed protein; further, in particular advantageous embodiments of this aspect are subject-matter of the respective dependent claims.

With respect to the aspects of the present invention it has to be pointed out that explanations, which have been made in relation to one aspect self-evidently also apply with respect to the other aspects.

Apart from this, a person skilled in the art can—depending on the application or depending on the individual case—deviate from the specified weights, specified quantities and specified ranges that are stated below without departing from the scope of the present invention.

Moreover, all specified values or specified parameters or the like that are mentioned below can absolutely be ascertained or determined using normed or standardized or explicitly specified determination methods or else using determination or measurement methods familiar per se to a person skilled in the art in this field.

With this said, the present invention will now be elucidated in detail below:

The present invention therefore provides—according to a first aspect of the present invention—conjugated gold nanoparticles, preferably for the use in the treatment, in particular a non-viral gene therapy, of a monogenetic disorder resulting from a mutation in a gene coding for a liver-specific and/or liver-expressed protein, comprising:
(a) laser-ablated gold nanoparticles;
(b) polyethylenimine (PEI) and/or derivatives and/or salts thereof; and
(c) at least one nucleic acid molecule, especially a vector, comprising (i) a promoter, preferably a promoter directing gene expression in mammalian, especially human cells and (ii) a coding sequence containing a nucleic acid sequence coding for a liver-specific and/or liver-expressed protein and/or preferably physiologically active domains and/or fragments thereof.

On the basis of the present invention conjugated gold nanoparticles have been developed which are suitable for the use in a novel non-viral gene therapy approach, wherein the transfection efficiency and/or the gene transfer efficiency is surprisingly improved on the basis of the use of laser-ablated gold nanoparticles. Furthermore, the use of laser-ablated gold nanoparticles is linked with a lowered toxicity and immunogenicity, when compared to the use of chemically synthesized gold nanoparticles. The conjugated gold nanoparticles according to the present invention are suitable for the transfer of intact copies of any coding sequence containing a nucleic acid sequence coding for a liver-specific and/or liver-expressed protein in order to allow the production of a therapeutically effective amount of the protein in the transfected cells.

In particular, the conjugated gold nanoparticles are suitable for the use in a novel gene therapy for hemophilia, allowing for a therapeutically effective production of the missing blood clotting factors in the patients, preferably coagulation factors VIII and/or IX. Nevertheless, the concept according to the present invention is suitable for the transfer of any other liver-specific and/or liver-expressed protein, in particular liver-specific and/or liver-expressed proteins which are associated with a monogenetic disorder.

The conjugated gold nanoparticles according to the present invention are linked with several advantages over known therapeutic concepts for the treatment of monogenetic disorders, in particular hemophilia:

According to the present invention it was surprisingly found that the use of laser-ablated gold nanoparticles as carrier material or carrier system is linked with several advantages when compared with known genetic approaches for the treatment of monogenetic disorders, in particular viral gene transfer systems, on the one hand, and chemically sympathized gold nanoparticles, on the other hand.

The use of gold nanoparticles obtained by laser ablation, in particular pulsed laser ablation in liquid, leads to an improved transfection efficiency, i.e. a higher transfection rate of target cells, as well as an efficient endosomal release of the nucleic acid molecules, in particular the vector, after cellular uptake. Furthermore, gold nanoparticles obtained by laser ablation are linked with a lesser toxicity and immunogenicity, in particular when compared to chemically synthesized gold nanoparticles. Overall, the use of gold nanoparticles obtained by laser ablation in the conjugated nanoparticles according to the present invention is safer when compared to approaches on the basis of viral vectors and chemically synthesized gold nanoparticles, on the one hand, and linked with an improved therapeutic efficacy, on the other hand. Furthermore, in contrast to chemically synthesized particles, undesired reactions, in particular the formation of agglomerates, can be prevented.

Without being bound to this theory, the advantages over chemically synthesized particles might be a result of the following physico-chemical properties of laser-ablated gold nanoparticles: In contrast to chemically synthesized gold nanoparticles, gold nanoparticles obtained by laser ablation do not contain any organic residues, in particular there is no need of stabilizing agents on the basis of citrate. Furthermore, the particles are free of gold-thiol bonds, which are necessary in chemically synthesized particles in order to achieve a stable binding of the transfection agent and the nucleic acid molecules bound thereto. Since the laser-ablated gold nanoparticles are essentially free of any organic residues, the particles comprise a freely accessible gold surface which leads to a higher carrier capacity with respect to the transfection agent, on the one hand, and the nucleic acid molecules to be transferred, on the other hand. Therefore, an improved loading with transfection agent and nucleic acid molecules resulting in a higher transfection efficiency is achieved.

Overall, it was not foreseeable at all that the use of laser-ablated gold nanoparticles is linked with the aforementioned advantageous effects when used as conjugated gold nanoparticles for the delivery of a gene coding for a liver-specific and/or liver-expressed protein. In this context, reference is also made to the working examples which show that laser-ablated gold nanoparticles lead to superior properties in comparison to chemically synthesized gold nanoparticles.

The conjugated gold nanoparticles according to the present invention are a promising candidate for the use in a therapeutic concept for the treatment of a variety of monogenetic disorders in order to introduce an intact copy of the mutated and/or deficient gene into the target cells. In this context, the conjugated gold nanoparticles are suitable for the transfection of liver cells.

In particular, the conjugated gold nanoparticles according to the present invention are suitable for the treatment of monogenetic disorders, particularly but not exclusively associated with an impaired and/or reduced hemostasis and/or blood clotting, especially wherein the disorder is a hemophilia, in particular hemophilia A and/or hemophilia B. Furthermore, the conjugated gold nanoparticles according to the present invention are suitable for the treatment of monogenetic lipometabolic disorders.

In other words, the conjugated gold nanoparticles according to the present invention are suitable to provide a long-term expression of the liver-specific and/or liver-expressed protein in the target cells, in particular liver cells. On this basis, it is possible to achieve an excellent efficacy of a therapeutic concept on the basis of the conjugated gold nanoparticles according to the present invention. When compared to genetic approaches known in the prior art as well as delivery systems on the basis of chemically synthesized gold nanoparticles, the concept according to the present invention is not only linked with an improved efficacy, but also with an improved safety, a lowered toxicity and a reduced number of required treatment units due to the highly efficient long-term expression of the liver-specific and/or liver-expressed protein.

Prior to further specifications of particularly preferred embodiments of the present invention, relevant definitions of terms used according to the present invention are given with respect to a better understanding of the claimed subject-matter:

The term “monogenetic disorder”, “monogenetic disease” or “single-gene disorder” refers to diseases or disorders, which result from modifications, in particular mutations, in a single gene occurring in all cells of the preferably human body. The mutations are in general linked with a partial or complete loss of the physiological function of the protein (“loss-of-function-mutation”). In particular, monogenetic disorders can result from sex-linked, recessive or dominant heredity. Furthermore, monogenetic disorders can result from sporadic mutations in a single gene.

According to the present invention, the term “nanoparticle” refers to particles having an average particle diameter between 1 and 100 nm. Nanoparticles according to the present invention are based on inorganic material, preferably ligand-free gold. Nanoparticles of this kind are particularly suitable for medical purposes, especially for the transfer and/or delivery of nucleic acid molecules, since they are substantially chemically inert. Surprisingly, on the basis of the present invention, gold nanoparticles have turned out as particularly well-suited carriers for nucleic acid molecules comprising nucleic acid sequences coding for liver-specific and/or liver-expressed proteins due to their non-toxicity and excellent biocompatibility, on the one hand, and their transfection efficiency, in particular with respect to liver cells, on the other hand. Gold nanoparticles are well tolerated in various mammals. After intravenous injection, they are preferably taken up by the liver and then excreted again via the bile.

“Laser ablation” in the sense of the present invention indicates a process of removing material from a solid surface, in particular gold, by irradiating the solid with a laser beam. According to a preferred embodiment of the present invention, removing of the material is performed with a pulsed laser, preferably by pulsed laser ablation in liquid (PLAL). The principal of pulsed laser ablation in liquid is based on focusing a laser beam on a solid target for ablation, in particular gold. The properties of the resulting particles, in particular the size, are controlled by the laser parameters used as well as solvent, temperature, pressure or wave length, pulse duration, energy or reputation rate. In general, the skilled practitioner is able to adapt the settings of the laser ablation to produce gold nanoparticles with the desired properties, in particular an appropriate size/diameter.

The term “polyethylenimine”, synonymous also “PEI”, “poly[imino(1,2-ethanediyl)]” a “polyaziridine”, as used according to the present invention, especially refers to a polycationic polymer with repeating units of an amine group and two carbon aliphatic CH₂CH₂ as a spacer between the repeating units of the amine groups. The chemical name of this polymer according to IUPAC is poly(iminoethylene). Linear polyethylenimines contain all secondary amines, wherein branched polyethylenimines contain primary, secondary and tertiary amino groups. Polyethylenimine was one of the first discovered transfection agents. When used as transfection agent—without being bound to this theory—, polyethylenimine condenses DNA into positively charged particles, which bind to anionic cell surface residues. The complex on the basis of DNA and polyethylenimine is then brought into the cell via endocytosis. Subsequently, the polyethylenimine causes an influx of water molecules into the endosomes, resulting in a bursting of the endosomes and a release of the DNA into the cytoplasm. According to the present invention, it was surprisingly found that polyethylenimines are not only suitable for the mediation of transfection as such, but also as a ligand for gold nanoparticles in order to build a gold nanoparticle/PEI/DNA complex. With respect to further information concerning polyethylenimine, reference is made to the encyclopedia RÖMPP Chemielexikon, 1999, 10^{th} edition, Georg Thieme Verlag Stuttgart, New York, page 3448, key word “polyethylenimine”.

Examples for variants of polyethylenimine for the delivery system according to the present invention are commercially available from Sigma-Aldrich Chemie GmbH, Munich, DE (branched PEI, 25 kDa), Polysciences Inc., Warrington, US (linear PEI, 10 kDa; linear PEI, 25 kDa; linear PEI, commercially available under the tradename Transporter5™) and/or Polyplus Inc., Illkirch, FR (JetPEI™, linear PEI, JetPEI™-Hepatocyte, galactose-conjugated linear PEI).

The term “vector” is used for a DNA molecule which is suitable for the use as a vehicle to artificially carry foreign genetic material, in particular genetic material comprising a nucleic acid sequence coding for a liver-specific and/or liver-expressed protein and/or preferably physiologically active domains and/or fragments thereof, into target cells. According to a preferred embodiment of the present invention, the vector used in the conjugated gold nanoparticles is a non-viral or mini circle vector in order to improve the safety and compatibility when used in gene therapy. Particularly, the vector used according to the present invention does not integrate into the genome. Thereby, the vector used according to the present invention still provides for a efficient transfection of the target cells and allows for a long-term expression of the coding sequence, preferably on the basis of an episomal attachment to the chromosomal DNA. In contrast to known approaches with respect to gene therapy for the treatment of monogenetic disorders, the vector used according to the present invention is not a viral vector, in particular no vector on the basis of the adeno-associated virus (AAV).

The term “promoter” as used according to the present invention relates to a DNA (desoxyribonucleic acid) or nucleic acid sequence, in particular a regulatory sequence, which is required for the expression of a coding sequence linked to the promoter, in particular a corresponding coding sequence located 3′ or downstream to the promoter. In order to achieve a stable and reliable expression of the nucleic acid sequence coding for a liver-specific and/or liver-expressed protein, the nucleic acid molecules, in particular the vector, comprise preferably a promoter derived from a eukaryotic, in particular human gene or a promoter derived from a virus. On this basis, the compliance of the conjugated gold nanoparticles with the nucleic acid molecules, in particular the vector, on the one hand, in the patient and the efficiency of expression of the coding sequence, on the other hand, can be improved. A promoter according to the present invention can comprise a core promoter, including a transcription start site, a binding site for RNA polymerases and binding sites for general transcription factors.

The term “coding sequence”, “coding region” or “nucleic acid coding sequence” refers to a nucleic acid sequence coding for a protein or domains or fragments of a protein. Furthermore, the coding sequence can refer to a nucleic acid sequence coding for fusion proteins, in particular fusion proteins on the basis of a liver-specific and/or liver-expressed protein and an albumin. In other words, the coding sequence according to the present invention contains a nucleic acid sequence coding for a liver-specific and/or liver-expressed protein and/or domains and/or fragments thereof and can contain further nucleic acid sequences, which results in a coding sequence coding for a fusion protein. In particular, according to a preferred embodiment of the present invention, the coding sequence is based on the cDNA sequence coding for a protein and/or domains or fragments of a protein.

In the following, particularly preferred embodiments of the present invention are delineated:

According to the present invention it is preferred when the laser-ablated gold nanoparticles are obtained by pulse laser ablation in liquid (PLAL). The use of laser-ablated gold nanoparticles in the conjugated gold nanoparticles according to the present invention is linked with several advantages, in particular with respect to the efficacy of gene transfer and a safe application without undesired side effects or adverse reactions, as delineated before.

With respect to the production of the laser-ablated gold nanoparticles as such, i.e. the particles before conjugation or “naked” particles, it is particularly preferred to use pulsed laser irradiation with a wave length in the range from 3.300 to 1.500 nm, preferably in the range from 800 to 1.200 nm. On this basis, particles with a suitable size and an even particle size distribution are obtained. With respect to further information regarding the laser ablation, reference can also be made to the third aspect of the present invention, which relates to the method for the preparation of conjugated gold nanoparticles.

Furthermore, in this context it is preferred when the gold nanoparticles before conjugation, i.e. the naked particles, have an average particle diameter dₚ [nm] in the range from 0.01 to 100 nm, in particular 0.05 to 80 nm, preferably 0.1 to 50 nm, particularly preferred 0.5 to 30 nm, even more preferred 1 to 15 nm, especially preferred 2 to 10 nm, preferably determined by analytical disc centrifugation (ADC) and/or transmission electron microscopy (TEM) and/or UV/VIS spectra.

Particularly reliable results with respect to the determination of the particle size are obtained by analytical disc centrifugation. Analytical disc centrifuge is an analytical device that can accurately determine the size distribution of colloidal systems. The method is particularly suitable for microscopic to submicroscopic spherical particles with sizes between 3 nm and 100 μm. The analysis of the particles is based on the sedimentation principle, in which a separation by different radii of the particles takes place upon penetration of a liquid medium. Regarding particles of the same density, the larger particles sediment faster than the smaller particles. If spherical bodies are used, the sedimentation rate can be determined by the Stokes equation.

Further information with respect to the determination of the particle diameter of the gold nanoparticles on the basis of analytical disc centrifugation and/or transmission electron microscopy are evident from the scientific publication of Fissan et al.: “Comparison of different characterization methods for nanoparticle dispersions before and after aerosolization”, published in Anal. Methods, 2014, 6: 7324-7334, the disclosure of which is hereby incorporated by reference. With respect to the determination of the particle diameter of the gold nanoparticles by UV/VIS spectra, further information are evident from the scientific publication of Haiss et al.: “Determination of Size and Concentration of Gold Nanoparticles from UV-Vis Spectra”, published in Anal. Chem., 2007, 79(11), 4215-4221, wherein the disclosure of the publication, in particular with respect to the details of the determination methods, is hereby incorporated by reference.

The particle size is adjusted by variation of laser energy, wavelength of the pulsed laser irradiation and time. The adjustment of the particle as such is performed on the basis von general knowledge of the skilled practitioner. Preparation of the gold nanoparticles by pulsed laser ablation in liquid can be performed by using a picosecond laser (commercially available from Ekspla, Vilnius, Lithuania).

In particular, the uptake of the gold nanoparticles by the cells can be significantly increased on the basis of the use of gold nanoparticles having the aforementioned size. Furthermore, a purposeful selection of the size and/or average particle diameter is relevant with respect to avoid the potential toxicity of gold nanoparticles. In particular, gold nanoparticles with a size below the aforementioned ranges behave different in cells leading to a certain toxicity. Gold nanoparticles having a size above the aforementioned ranges, however, are not able to penetrate the cell membrane and are therefore not suitable for a transfer of nucleic acid molecules. The use of gold nanoparticles having the aforementioned sizes leads to an efficiency enhancement with respect to the transfection efficiency, on the one hand, and a reduced, preferably non-existent toxicity—in other words an improved biocompatibility—with respect to the cells.

With respect to the “naked”, i.e. the unconjugated gold nanoparticles, it is advantageous when the gold nanoparticles before conjugation have a gold surface, wherein at least 90%, preferably at least 95% of said gold surface is freely accessible and not attached to any molecules. On this basis, the loading capacity of the gold nanoparticles with nucleic acid molecules and transfection agent is improved. As a result, the transfection efficiency as such as well as the DNA transfer, in particular the endosomal release of the nucleic acid molecules after uptake by the cell, are further improved.

Furthermore, with respect to the particle size of the conjugated particles, it is advantageous when the conjugated gold nanoparticles have an average hydrodynamic diameter d_{hd} [nm] in the range from 0.05 to 150 nm, in particular 0.1 to 100 nm, preferably 0.5 to 80 nm, particularly preferred 1 to 50 nm, even more preferred 2 to 40 nm, especially preferred 10 to 30 nm, preferably determined by the method of dynamic light-scattering.

With respect to the determination of the average hydrodynamic diameter of the conjugated nanoparticles, known methods for the measurement of the hydrodynamic diameter of nanoparticles are used, in particular dynamic light scattering. With respect to the determination of the hydrodynamic radius of the conjugated gold nanoparticles by dynamic light scattering, reference can be made to the publication according to Menendez-Manjon and Barcikowski: “Hyrodynamic size distribution of gold nanoparticles controlled by repetition rate during pulsed laser ablation in water”, published in Appl. Surf. Sci., Vol. 257, Issue 9, 2011.

In order to provide an efficient transfection, on the one hand, and a stable binding of the nucleic acid molecules, in particular the vector, on the other hand, the polyethylenimine and/or derivatives and/or salts thereof are bound to the gold nanoparticles, preferably through electrostatic interaction with the surface of the gold nanoparticles. In particular and without being bond to this theory, it is assumed that the electrostatic interaction is based on partial charges of the nitrogen atoms of the polyethylenimine, on the one hand, and the gold nanoparticles, on the other hand. The respective single electrostatic bonds are rather weak, but in total, i.e. the sum of all bonds, a bonding of the polyethylenimine to the gold nanoparticles is achieved, which is strong enough in order to provide stable conjugated gold nanoparticles but thereby still allowing an endosomal release of the nucleic acid molecules, in particular of the vector, after uptake by the cell.

With respect to the transfection agent, it is particularly preferred when the polyethylenimine and/or derivatives and/or salts thereof are selected from the group of (i) linear polyethylenimines and/or derivatives and/or salts thereof; (ii) branched polyethylenimines and/or derivatives and/or salts thereof; and/or (iii) monosaccharide-conjugated, preferably galactose-conjugated polyethylenimines and/or derivatives and/or salts thereof.

Polyethylenimine is a particularly efficient transfection agent with respect to the conjugated gold nanoparticles according to the present invention since it is highly compatible and linked with a high loading capacity with respect to the nucleic acid molecules, resulting in an efficient DNA transfer. Particularly good results with respect to compatibility and non-toxicity and furthermore with respect to transfection efficiency can be achieved with the use of linear polyethylenimines. Furthermore, the use of a monosaccharide-conjugated polyethylenimine, preferably galactose-conjugated polyethylenimine, is linked with an additional function of the polyethylenimine. For, on this basis a targeting of the conjugated gold nanoparticles is possible. In particular liver cells comprise in their membrane galactose specific cell surface receptors, for example galactose-specific membrane lectins as asialoglycoprotein receptors (ASGPR). By the use of a polyethylenimine conjugated with galactose, the conjugated gold nanoparticles can specifically bind to the respective receptors in the cell surface of liver cells, followed by an uptake of the conjugated gold nanoparticles by the cells. On this basis, the specificity of the conjugated gold nanoparticles according to the present invention can be further improved. Galactose conjugated polyethylenimine is commercially available from Polyplus Inc., Illkirch, FR under the tradename “JetPEI®-hepatocyte”.

Furthermore, according to a particularly preferred embodiment of the present invention, the conjugated gold nanoparticles comprise at least two layers of polyethylenimine and/or derivatives and/or salts thereof. With respect to this embodiment, it is particularly preferred when the conjugated gold nanoparticles comprise the at least two layers of polyethylenimine in the sense of a layer-by-layer assembly, i.e. an inner layer on the basis of polyethylenimine, wherein nucleic acid molecules are bound to this inner layer of polyethylenimine. The gold nanoparticles conjugated with said inner layer and nucleic acid molecules bound thereto are further conjugated with a second polyethylenimine layer and/or an outer layer on the basis of polyethylenimine.

In particular, the conjugated gold nanoparticles comprise alternating layers of polyethylenimine and/or derivatives and/or salts thereof and nucleic acid molecules, in particular an inner and an outer layer comprising polyethylenimine and/or derivatives and/or salts thereof, wherein nucleic acid molecules are bound to the inner and/or the outer layer.

With respect to the embodiment of the conjugated gold nanoparticles according to the present invention comprising of a layer-by-layer assembly, it is particularly intended that the inner layer comprises linear and/or branched, preferably linear polyethylenimines and/or derivatives and/or salts thereof. On this basis, the surface of the gold nanoparticles to be conjugated is covered with a sufficient amount of transfection agent providing a good loadability of the particles with nucleic acid molecules. After loading and/or conjugating the inner layer with nucleic acid molecules, the conjugated gold nanoparticles are covered or coated with an outer layer, also on the basis of polyethylenimines and/or derivatives and/or salts thereof. In this context, it is particularly preferred when the outer layer comprises linear, branched and/or monosaccharide-conjugated, preferably monosaccharide-conjugated polyethylenimines and/or derivatives and/or salts thereof.

On the basis of an outer layer and/or a layer-by-layer assembly of the polyethylenimine and the nucleic acid molecules, the transfection efficiency and the transfer of nucleic acid molecules is further improved. Furthermore, on the basis of the use of monosaccharide-conjugated polyethylenimines, in particular galactose-conjugated polyethylenimines, in the outer layer a specific targeting of the gold nanoparticles to liver cells is achieved.

Furthermore, the transfection efficiency and compatibility of the delivery system according to the present invention can be further improved on the basis of the use of polyethylenimines and/or derivatives and/or salts thereof having a defined number average molecular weight. In particular, it is preferred when the polyethylenimine and/or derivatives and/or salts thereof have a number average molecular weight Mₙ in the range from 10 Da to 200 kDa, in particular from 100 kDa to 150 kDa, especially from 1 kDa to 100 kDa, particularly from 2 kDa to 50 kDa, preferably from 5 kDa to 40 kDa, more preferably from 8 kDa to 30 kDa, for example determined by means of gel permeation chromatography and/or according to DIN 55672-3:2016-03. In this context, reference is made to the working examples performed by the applicant, which show that the purposeful selection of polyethylenimine and/or derivatives and/or salts thereof having a certain molecular weight leads to an improved transfection efficiency as well as a reduced toxicity.

With respect to a particularly compatible therapeutic concept with a lowered risk of undesired side effects, in particular an undesired integration of the nucleic acid molecules into the gene known, it is preferred that the vector is a non-viral and not integrating vector. In other words, the conjugated gold nanoparticles according to the present invention are designed for a non-viral approach with respect to transfection and gene delivery. In particular, the conjugated gold nanoparticles are free from vectors on the basis of adeno-associated viruses (AAV), lentiviruses, retroviruses, adenoviruses and hybrids on the basis of the aforementioned vector systems. In particular, this means that the transfection mechanisms used according to the present invention is not based on viral systems. It is still possible though that the vectors used in the conjugated gold-nanoparticles comprise promoter sequences or elements of viral origin for the regulation of transcription.

In order to provide a stable and/or specific expression of the coding sequence contained in the nucleic acid molecule, it is preferred when the promoter is inducible and/or constitutive in mammalian cells, in particular human cells, preferably liver cells and/or fibroblasts. Likewise, it is possible that the promoter directs a tissue-specific, in particular liver-specific expression of the coding sequence. On this basis, the specificity of the promoter or the specificity of the expression directed by the promoter is variable and can be purposefully tailored or adjusted. In particular, any promoter directing a preferably constitutive expression of the coding sequence in several mammalian cells, cell types or tissues can be used in the nucleic acid molecules in the conjugated gold nanoparticles according to the present invention. Likewise, on the basis of tissue-specific promoters, in particular liver-specific promoters, the expression of the coding sequence can be purposefully targeted or adjusted. In this context, the promoter can be tailored and/or selected depending on the target cells, the severeness of the monogenetic disorder and the coding sequence to be expressed.

According to the present invention, the specificity of the promoter or the specificity of the expression directed by the promoter is variable and can be purposefully tailored or adjusted. In particular, any promoter directing a preferably constitutive expression of the coding sequence in several mammalian cells, cell types or tissues can be used in the nucleic acid molecules, in particular the vectors. In particular, in connection with the expression of coding sequences having nucleic acid sequence coding for a protein involved in hemostasis, the use of a constitutively active promoter is preferred.

According to a preferred embodiment of the present invention, the promoter is derived from the gene coding for human Elongation Factor-1 alpha (EF1a). In particular, according to a further preferred embodiment of the present invention, the promoter is derived from the promoter of the gene coding for human Elongation Factor-1 alpha (EF1a) and the first intron and/or a fragment of the first intron of the gene coding for human Elongation Factor-1 alpha (EF1a). A promoter derived from human Elongation Factor-1 alpha (EF1a) directs a reliable and constant expression of the coding sequences in mammalian cells, in particular human cells, preferably liver cells and/or fibroblasts, especially hepatocytes and/or fibroblasts. In this context, reference is also made to the working examples performed by the applicant. The working examples performed by applicant show that different promoters derived from the gene coding for human Elongation Factor-1 lead to a stable long-term expression of the coding sequence in several cell types, for example liver cells or fibroblasts (cf. also working examples).

Furthermore, according to another preferred embodiment of the present invention, the promoter is derived from the human SERPINA1 promoter. The SERPINA1 promoter directs a reliable and constant expression of the coding sequences in mammalian cells, in particular human cells, preferably liver cells and/or fibroblasts.

According to another, likewise preferred embodiment of the present invention, the promoter is derived from the hAAT (human alpha1-antitrypsin) promoter. The use of this promoter is particularly suitable with respect to directing a constant and stable expression of the coding sequence in mammalian cells, in particular human cells, preferably liver cells and/or fibroblasts. In this context, reference can also be made to the working examples performed by the applicant. On the basis of the working examples, it can be seen that the hAAT promoter leads to a stable long-term expression of the coding sequence in various cell types, in particular liver cells or fibroblasts.

According to another preferred embodiment of the present invention, the promoter is derived from Cytomegalovirus (CMV), in particular human CMV. In other words, according to this embodiment of the present invention, the promoter is the CMV promoter. The CMV promoter directs a stable and reliable gene expression in several mammalian cell types, for examples liver cells, in particular hepatocytes, or fibroblasts. With respect to the expression level of the coding sequence, reference is made to the working examples performed by applicant, which verify the stable expression of the coding sequence under control of the CMV promoter.

Furthermore, according to the present invention it can be intended that the promoter comprises a codon-optimized nucleic acid sequence and/or a nucleic acid sequence optimized for human gene expression and/or human codon usage. In particular, this applies for embodiments with a promoter containing further regulatory elements, for example on the basis of introns or parts of introns of a gene, especially of the gene the promoter is derived from.

According to a preferred embodiment of the present invention, the promoter has a nucleotide sequence according to SEQ ID NO. 1, SEQ ID NO. 2, SEQ ID NO. 3, SEQ ID NO. 4 and/or SEQ ID NO. 5, preferably SEQ ID NO. 2, SEQ ID NO. 3 and/or SEQ ID NO. 4. Likewise, according to a preferred embodiment of the present invention, the promoter has a nucleic acid sequence having at least 85%, in particular at least 90%, preferably at least 95% identity with SEQ ID NO. 1, SEQ ID NO. 2, SEQ ID NO. 3, SEQ ID NO. 4 and/or SEQ ID NO. 5, preferably SEQ ID NO. 2, SEQ ID NO. 3 and/or SEQ ID NO. 4.

Particularly preferred promoter sequence contained in the nucleic acid molecules used in the conjugated gold nanoparticles according to the present invention is derived from the gene, in particular the promoter, of human Elongation Factor-1 alpha (EF1a). According to a preferred embodiment of the present invention, the promoter on the basis of EF1a contains a sequence optimized first intron, which has been considerably shortened. Furthermore, a cryptic splice side contained in the native nucleotide sequence has been deleted. The promoter according to SEQ ID NO. 2 and/or SEQ ID NO. 3 leads to a stable and highly efficient expression of the coding sequence in mammalian cells.

Likewise, according to another particularly preferred embodiment of the present invention, the nucleic acid molecules comprise the hAAT promoter in order to direct the expression of the coding sequence. In this context, it is preferred when the promoter has a nucleic acid sequence according to SEQ ID NO. 4.

In order to further enhance the expression of the coding sequence, it can be intended that the nucleic acid molecules, in particular the vector, contain at least one further cis-regulatory element, especially at least one further transcriptional enhancer.

According to a preferred embodiment of the present invention, the cis-regulatory element is derived from the apolipoprotein E gene, in particular the apolipoprotein E hepatic locus control region. The additional use of a cis-regulatory element on the basis of the apolipoprotein E hepatic locus control region (HCR) leads to an improved expression of the coding sequence in the target cells.

In this context, it is particularly preferred when the cis-regulatory element has a nucleotide sequence according to SEQ ID NO. 6 and/or when the cis-regulatory element has a nucleic acid sequence having at least 85%, in particular at least 90%, preferably at least 95% identity with SEQ ID NO. 6.

In particular, a further cis-regulatory element has been proven to be advantageous with respect to the expression efficiency when used together with a SERPINA1 promoter or a hAAT promoter.

A preferred design of the coding sequence contained in the nucleic acid molecules, in particular the vector, according to the present invention is delineated in the following:

In order to achieve an improved expression of the coding sequence, according to the present invention it is intended that the nucleic acid sequence of the coding sequence is codon-optimized for human gene expression and/or human codon usage. The introduction of synonymous mutations, i.e. mutations that lead to the same translational product, leads to an efficient enhancement of the protein expression. On the basis of a replacement of rare codons with preferred codons, the expression of the coding sequence and the production of the target protein in the target cells can be further improved.

With respect to the selection of the coding sequence, according to a preferred embodiment of the present invention, the coding sequence comprises a nucleic acid sequence coding for a liver-specific and/or liver-expressed protein selected from proteins produced and/or predominantly expressed in the liver. As delineated before, the production and secretion of proteins belong to the main functions of the liver. The proteins produced and secreted by the liver in particular include proteins involved in hemostasis, i.e. proteins regulating blood clotting. Mutations in genes coding for liver-specific and/or liver-expressed proteins can lead to a reduced or completely lacking production of the protein. Furthermore, mutations can result in the production of defective proteins, i.e. proteins that lost their physiological functionality (so called lost-of-function-mutation).

Factors involved in hemostasis and fibrinolysis are of particular importance for the present invention, since mutations in genes coding for such factors or proteins, in particular factors of the coagulation cascade, lead to a group of monogenetic disorders subsumed as hemophilia. Liver-specific and/or liver-expressed proteins involved in hemostasis and fibrinolysis are in particular all factors of the coagulation cascade, especially fibrinogen (FI), prothrombin (FII), tissue factor or tissue thromboplastin (FIII), proaccelurin or labile factor (FV), stable factor or proconvertin (FVII), antihemophilic factor A (FVIII), antihemophilic factor B, synonymously also known as Christmas factor (FIX), Stuart-Prower factor (FX), plasma thromboplastin antecedent (FXI), Hageman factor (FXII), fibrin-stabilizing factor (FXIII), von Willebrand factor (VWF), Fletcher factor, synonymous also prekallicrein, high-molecular weight kininogen or Fitzgerald factor, fibronectin, antithrombin III, heparin-co-factor II, protein-C, protein-S, protein-Z, plasminogen, alpha2-antiplasmin, tissue plasminogen activator, urokinase and plasminogen activator inhibitor-1 (PAI1). Mutations in genes coding for the aforementioned coagulation factors and related substances can lead to genetic disorders, in particular to different types or subforms of hemophilia.

Further liver-specific and/or liver-expressed proteins of particular interest with respect to the present invention are proteins of the amino acid metabolism, in particular fumarylacetoacetate hydrolase, p-hydroxyphenylpyruvate hydroxylase and/or phenylalanine-4-hydroxylase, antiproteases, in particular alpha-1 antitrypsin, proteins of the bilirubin metabolism, in particular uridine diphospho-glucuronosyltransferase, proteins of the urea cycle, in particular arginase, argininosuccinate synthase and/or ornithine transcarbamylase, proteins of the carbohydrate metabolism, in particular alpha-glucan phosphorylase, amylo-1,6-glucosidase and/or glucose-6-phosphatase, proteins of the proteoglycan metabolism, in particular idursulfase, proteins of the sphingolipid metabolism, in particular glucocerebrosidase, and/or proteins involved in transport processes, in particular p-type ATPase, cystic fibrosis transmembrane regulator and/or low-density lipoprotein (LDL) receptor.

According to a preferred embodiment of the present invention, the coding sequence has a nucleic acid sequence coding for a human liver-specific and/or liver-expressed protein selected from the group of:
(i) major plasma proteins, in particular human serum albumin, alpha-fetoprotein, soluble plasma fibronectin, C-reactive protein and/or preferably physiologically active domains and/or fragments thereof;
(ii) stimulators and/or factors for coagulation, preferably coagulation factor FVII, FVIII, FIX, FX, FXI, FXII, FXIII and/or preferably physiologically active domains and/or fragments thereof, preferably FVIII, FIX and/or preferably physiologically active domains and/or fragments thereof;
(iii) inhibitors of coagulation, preferably alpha2-macroglobulin, alpha1-antitrypsin, antithrombin III, protein S, protein C and/or preferably physiologically active domains and/or fragments thereof;
(iv) stimulators of fibrinolysis, preferably plasminogen and/or preferably physiologically active domains and/or fragments thereof; and/or
(v) inhibitors of fibrinolysis, preferably alpha2-antiplasmin and/or preferably physiologically active domains and/or fragments thereof; and/or
(vi) proteins of the amino acid metabolism, in particular fumarylacetoacetate hydrolase, p-hydroxyphenylpyruvate hydroxylase and/or phenylalanine-4-hydroxylase; and/or
(vii) antiproteases, in particular alpha-1 antitrypsin; and/or
(viii) proteins of the bilirubin metabolism, in particular uridine diphospho-glucuronosyltransferase; and/or
(ix) proteins of the urea cycle, in particular arginase, argininosuccinate synthase and/or ornithine transcarbamylase; and/or
(x) proteins of the carbohydrate metabolism, in particular alpha-glucan phosphorylase, amylo-1,6-glucosidase and/or glucose-6-phosphatase; and/or
(xi) proteins of the proteoglycan metabolism, in particular idursulfase; and/or
(xii) proteins of the sphingolipid metabolism, in particular glucocerebrosidase; and/or
(xiii) proteins involved in transport processes, in particular p-type ATPase, cystic fibrosis transmembrane regulator and/or low-density lipoprotein (LDL) receptor; and/or
(xiv) proteins involved in lipometabolism and/or proteins linked with monogenetic lipometabolic disorders.

In particular, mutations in genes coding for coagulation factors are associated with genetic disorders, which are commonly summed up as hemophilia, in particular hemophilia A (factor FVIII deficiency), hemophilia B (factor FIX deficiency), von Willebrand disease (von Willebrand factor deficiency) and the rare factor deficiencies including deficiencies in factor FI, FII, FV, FVII, FX, FXI, FXII and/or FXIII. The conjugated gold nanoparticles with the nucleic acid molecules, in particular the vectors, can be used to transfer an intact copy of the genes coding for coagulation factors into the target cells, in particular liver cells. On this basis, the physiological deficiency with respect to respective coagulation factor can be balanced and/or improved through the stable expression of the coding sequence in the target cells, in particular liver cells.

It is especially preferred when the coding sequence has a nucleic acid sequence coding for a coagulation factor, in particular coagulation factor FVII, FVIII, FIX, FX, FXI, FXII, FXIII and/or preferably physiologically active domains and/or fragments thereof, preferably coagulation factor FVIII, FIX and/or preferably physiologically active domains and/or fragments thereof.

More particularly preferred is an embodiment of the present invention, wherein the coding sequence has a nucleic acid sequence coding for coagulation factor FVIII and/or preferably physiologically active domains and/or fragments thereof. In hemostasis, factor FVIII functions as cofactor for factor FIXa, which is necessary for the formation of factor FX. Mutations, in particular loss-of-function-mutations, in the gene coding for factor FVIII are linked with hemophilia A.

According to a particularly preferred embodiment of the present invention, the coding sequence has a nucleic acid sequence coding for coagulation factor FVIII with a deleted B-domain. The native FVIII protein has a total length of 2.351 amino acids with the so-called B-domain constituting of 911 amino acids. The B-domain is a highly glycosylated region of the protein but is not required for the physiological procoagulation activity of FVIII. On the basis of the deletion of the B-domain and the replacement of the B-domain by a short 14 amino acid linker, a fully functional fragment of FVIII can be provided which shows—due to the reduction of the length—an improved expression in the target cells.

According to a likewise preferred embodiment of the present invention, the coding sequence has a nucleic acid sequence coding for coagulation factor FIX and/or preferably physiologically active domains and/or fragments thereof. The physiological function of factor FIX is, together with Ca²⁺, membrane phospholipids and a factor FVIII cofactor, the formation of factor FX. Mutations, especially loss-of-function-mutations, in the gene coding for coagulation factor FIX result in hemophilia B. Conjugated gold nanoparticles comprising a nucleic acid sequence coding for coagulation factor FIX are therefore suitable for the use in a gene therapy for the treatment of hemophilia B in order to balance the loss of function caused by the mutation.

According to a further preferred embodiment of the present invention, the coding sequence has a nucleic acid sequence coding for a fusion protein on the basis of a coagulation factor and/or preferably physiologically active domains and/or fragments thereof, in particular coagulation factor FVIII and/or FIX, preferably coagulation factor FIX, and an albumin and/or domains and/or fragments thereof. On the basis of a fusion of coagulation factors to albumin, the pharmacokinetic properties of the coagulation factors can be significantly improved. In particular, coagulation factors on the basis of fusions with albumin comprise an extended half-life time. On this basis, the treatment intervals of the patients suffering from monogenetic disorders, in particular hemophilia, can be prolonged, i.e. a less frequent dosing is enabled.

Nevertheless, the list of coding sequences is not exhaustive, since the nucleic acid sequences coding for any liver-specific and/or liver-expressed protein associated with a monogenetic disorder can be integrated into the nucleic acid molecules used in the conjugated gold nanoparticles.

According to a preferred embodiment of the present invention, the coding sequence has a nucleotide sequence coding for coagulation factor FVIII and/or preferably physiologically active domains and/or fragments thereof. According to a particularly preferred embodiment of the present invention the coding sequence has a nucleotide sequence according to SEQ ID NO. 7 and/or SEQ ID NO. 8, preferably SEQ ID NO. 8, and/or wherein the coding sequence has a nucleotide sequence having at least 85%, in particular at least 90%, preferably at least 95% identity with SEQ ID NO. 7 and/or SEQ ID NO. 8, preferably SEQ ID NO. 8. Likewise, the coding sequence can have a nucleic acid sequence corresponding to the nucleic acid sequence of the native cDNA coding for human coagulation factor FVIII and/or the coding sequence can code for a protein having an amino acid sequence according to SEQ ID NO. 9 and/or an amino acid sequence having at least 85%, in particular at least 90%, preferably at least 95% identity with SEQ ID NO. 9.

According to a likewise preferred embodiment of the present invention, the coding sequence comprises a nucleic acid sequence coding for coagulation factor FIX and/or preferably physiologically active domains and/or fragments thereof. With respect to the nucleic acid molecules comprising a coding sequence for expressions of a protein, which carries out the physiologically functions of coagulation factor FIX, according to a preferred embodiment of the present invention the coding sequence has a nucleotide acid sequence according to SEQ ID NO. 10, SEQ ID NO. 11 and/or SEQ ID NO. 12 and/or a nucleotide sequence having at least 85%, in particular at least 90%, preferably at least 95% identity with SEQ ID NO. 10, SEQ ID NO. 11 and/or SEQ ID NO. 12. Likewise, the coding sequence can have a nucleotide sequence corresponding to the nucleotide sequence of the native cDNA coding for human coagulation factor FIX and/or wherein the coding sequence codes for a protein having an amino acid sequence according to SEQ ID NO. 13 and/or SEQ ID NO. 14 and/or an amino acid sequence having at least 85%, in particular at least 90%, preferably at least 95% identity with SEQ ID NO. 13 and/or SEQ ID NO. 14.

According to a further preferred embodiment of the present invention, the coding sequence has a nucleic acid sequence coding for a fusion protein on the basis of a coagulation factor and/or preferably physiologically active domains and/or fragments thereof, in particular coagulation factor FVIII and/or FIX, preferably coagulation factor FIX, and an albumin and/or domains an/or fragments thereof.

On the basis of a fusion of coagulation factors to albumin, the pharmacokinetic properties of the coagulation factors can be significantly improved. In particular, coagulation factors on the basis of fusions with albumin comprise an extended half life time. On this basis, the treatment intervals of the patience suffering from monogenetic disorders, in particular hemophilia, can be prolonged, i.e. a less frequent dosing leads to desired therapeutic effect.

In this context, according to a preferred embodiment of the present invention, the coding sequence has a nucleotide sequence according to SEQ ID NO. 15 and/or SEQ ID NO. 16 and/or a nucleotide sequence having at least 85%, in particular at least 90%, preferably at least 95% identity with SEQ ID NO. 15 and/or SEQ ID NO. 16. Likewise, the coding sequence can code for a protein having an amino acid sequence according to SEQ ID NO. 17 and/or SEQ ID NO. 18 and/or an amino acid sequence having at least 85%, in particular at least 90%, preferably at least 95% identity with SEQ ID NO. 17 and/or SEQ ID NO. 18.

Nevertheless, the list of coding sequences is not exhaustive, since the nucleic acid sequences coding for any liver-specific and/or liver-expressed protein associated with a monogenetic disorder can be integrated into the nucleic acid molecules, in particular the vectors, used according to the present invention.

As delineated before, the conjugated gold nanoparticles according to the present invention are designed in order to provide a non-viral genetic approach for the treatment of monogenetic disorders. In other words, the conjugated gold nanoparticles are designed in order to import an intact copy of a gene coding for a liver-specific and/or liver-expressed protein into the target cells, preferably liver cells or fibroblasts, in order to provide a therapeutically efficient expression of the protein. Since the nucleic acid molecules, in particular the vectors, contained in the conjugated gold nanoparticles do not integrate or insert into the genome, there is a possibility that the transfected cells lose the transferred nucleic acid molecules during the cell cycle. According to the present invention, it was found that the long-term expression of the coding sequence is improved with an increase of the episomal persistence of the nucleic acid molecule in the target cells. In this context it was surprisingly found that the episomal persistence is significantly improved when the vector comprises a scaffold/matrix attachment region, in particular a scaffold/matrix attachment region derived from the gene coding for human Interferon-beta (IFN-beta).

The term “scaffold/matrix attachment region”, also indicated as “S/MAR element” or “scaffold-attachment region” or “matrix-associated region”, refers to DNA sequences of eukaryotic chromosomes where the nuclear matrix attaches. Scaffold/matrix attachment regions of the eukaryotic DNA consist of about 70% T-rich regions and naturally mediate the structural organization of the chromatin within in the nucleus. In particular, the S/MAR elements constitute anchor points of the DNA for the chromatin scaffold and serve to organize the chromatin into structural domains. According to the present invention, it was surprisingly found that the use of the nucleotide sequence of a scaffold/matrix attachment region in the nucleic acid sequences, in particular the vectors, mediates the attachment of the transfected nucleic acid molecules to the nuclear matrix or the chromatin. On this basis, the non-integration of the nucleic acid molecules or the vector can be assured, thereby still allowing a stable expression of the coding sequence and a replication of the introduced nucleic acid molecule in particular during the S-phase of mitosis. The use of a scaffold/matrix attachment region increases the long-term episomal persistence of the nucleic acid molecules or the vector in the transfected target cells. Overall, the use of a nucleic acid sequence derived from a scaffold/matrix attachment region of a human gene is linked with a central advantage of the present invention, namely the prevention of an integration of the transferred transgenic nucleic acid molecules into the genomic DNA of the target cells. On this basis, the risk of further mutations, which can lead to the occurrence of malignant cells, can be significantly reduced.

In this context, it is particularly preferred when scaffold/matrix attachment region and for the SIMAR element has a nucleotide sequence according to SEQ ID NO. 19 and/or SEQ ID NO. 20, in particular SEQ ID NO. 20, and/or a nucleotide sequence having at least 85%, in particular at least 90%, preferably at least 95% identity with SEQ ID NO. 19 and/or SEQ ID NO. 20, in particular SEQ ID NO. 20. With respect to the assembly of the elements of the nucleic acid molecules, in particular the vector, it is preferred when the nucleic acid sequence derived from the scaffold/matrix attachment region of a eukaryotic gene is located 3′ to the promoter and/or the coding sequence.

The vector used according to the present invention can contain further elements advantageous or necessary for directing a stable expression of the coding sequence in the target cells. On the basis of the general knowledge, the skilled practitioner is able to select such further elements.

In particular, the vector can contain a transcription termination signal. The term “transcriptional termination signal” or “polyadenylation signal” as used according to the present invention refers to the section of a nucleic acid sequence that marks the end of a gene and/or a coding sequence during transcription. This sequence mediates the transcriptional termination by providing signals in the newly synthesized mRNA that trigger processes, which release the mRNA from the transcriptional complex. With respect to the present invention, the use of any transcriptional terminator suitable for the use in humans can be intended. The selection of a transcriptional termination signal and/or a polyadenylation signal does not represent a problem for the skilled practitioner.

Additionally, in order to optimally direct the expression of the coding sequence, the arrangement of the different elements of nucleic acid sequences within the nucleic acid molecules, in particular the vector, is of significance. In context with explanations concerning the assembly and/or arrangement of the nucleic acid sequence elements within the vector, the term “5′ to . . . ” is used synonymously to “upstream to . . . ”. Likewise, the term “3′ to . . . ” is used synonymously to “downstream to . . . ”. In other words, the terms upstream (“5′ to . . . ”) and downstream (“3′ to . . . ”) relate to the 5′ to 3′ direction in which RNA transcription takes place. In relation to double-stranded DNA, upstream is toward the 5′ end of the coding strand for the respective coding sequence and downstream is toward the 3′ end of the coding strand.

According to a preferred embodiment of the present invention, the promoter is located 5′ to the coding sequence and optionally the nucleic acid sequence derived from a scaffold/matrix attachment region of a human gene and/or a transcriptional termination signal. In particular, the elements, especially the promoter and the coding sequence, are arranged that the promoter can direct the expression of the coding sequence. Likewise, according to a preferred embodiment of the present invention, the optional nucleic acid sequence derived from the scaffold/matrix attachment region of a eukaryotic, in particular human gene is located 3′ to the promoter and/or the coding sequence. On this basis, a stable expression of the coding sequence and a high episomal persistence are provided.

With respect to a transcriptional termination signal in the vector, it is preferred when the transcriptional termination signal is located 3′ to the promoter and/or the coding sequence and/or optionally to a nucleic acid sequence derived from the scaffold/matrix attachment region of a human gene. As delineated before, the transcriptional termination signal is located such that the termination of the transcription of the coding sequence is enabled.

With respect to the transfection mediated by the conjugated gold nanoparticles according to the present invention it was found that transfection efficiency is not only influenced by gold nanoparticles, transfection reagent and nucleic acid molecules as such, but also by their proportions or ratios to one another, as delineated in the following:

In particular the transfer of nucleic acid molecules into the target cells can be improved on the basis of a defined weight related ratio of polyethylenimine to nucleic acid molecules. Particularly good results are achieved, when the weight related ratio of polyethylenimine to nucleic acid molecules is in the range of from 1:100 to 60:1, in particular from 1:50 to 40:1, especially from 1:30 to 20:1, preferably from 1:10 to 10:1, more preferred from 1:1 to 10:1, further preferred from 1:1 to 6:1. Likewise, it is preferred when the weight related ratio of polyethylenimine and/or derivatives and/or salts thereof to gold nanoparticles is in the range of from 1:100 to 100: 1, especially from 1:50 to 50:1, preferably from 1:30 to 20:1, in particular preferred from 1:20 to 10:1, even more preferred from 1:10 to 1:1.

With respect to the weight related ratios of the component of the delivery system according to the present invention, reference is also made to the working examples performed by applicant, which show that a purposefully selected weight related ratio leads to an improvement of the transfection efficiency and the resulting transfer of nucleic acid molecules into the target cells.

Overall, the conjugated gold nanoparticles according to the present invention are suitable for the use in the treatment, in particular a non-viral gene therapy, of a monogenetic disorder resulting from a mutation in a gene coding for a liver-specific and/or liver-expressed protein. In particular, the conjugated gold nanoparticles are able to transfer a intact copy of a gene coding for a liver specific and/or liver-expressed protein by transfection into the target cells, in particular mammalian cells, preferably human cells, for example liver cells of fibroblasts.

In context with the use of the gold nanoparticles according to the present invention it is particularly preferred when the monogenetic disorder is associated with an impaired and/or reduced hemostasis and/or blood clotting, especially wherein the disorder is a hemophilia, in particular hemophilia A and/or hemophilia B.

A further subject of the present invention is—according to a second aspect of the present invention—the use of conjugated gold nanoparticles as described before in the treatment, in particular a non-viral gene therapy, of a monogenetic disorder resulting from a mutation in a gene coding for a liver-specific and/or liver-expressed protein, and/or for the preparation of a medicament for the treatment, in particular a non-viral gene therapy, of a monogenetic disorder resulting from a mutation in a gene coding for a liver-specific and/or liver-expressed protein, preferably via transfection.

In this context, it is particularly preferred when the monogenetic disorder is associated with an impaired and/or reduced hemostasis and/or blood clotting, especially wherein the disorder is a hemophilia, in particular hemophilia A and/or hemophilia B.

For further details concerning this aspect of the invention, reference can be made to the above explanations in relation to the first inventive aspect, referring to the conjugated gold nanoparticles according to the present invention, said explanations also applying accordingly with regard to this aspect of the invention.

A further subject of the present invention is—according to a third aspect of the present invention—a method for the preparation of conjugated gold nanoparticles, wherein the gold nanoparticles comprise polyethylenimine (PEI) and/or derivatives and/or salts thereof, in particular conjugated gold nanoparticles as described before, and

wherein the method comprises the following method steps:
(a) providing unconjugated (naked) gold nanoparticles by laser ablation, especially pulsed laser ablation in liquid (PLAL);
(b) conjugating the gold nanoparticles with polyethylenimine (PEI) and/or derivatives and/or salts thereof; and
(c) conjugating the gold nanoparticles with nucleic acid molecules, especially a vector, comprising (i) a promoter, preferably a promoter directing gene expression in mammalian, especially human cells, and (ii) a coding sequence containing a nucleic acid sequence coding for a liver-specific and/or liver-expressed protein and/or preferably physiologically active domains and/or fragments thereof, wherein mutations in the nucleic acid sequence coding for the liver-specific and/or liver-expressed protein are associated with a monogenetic disorder, preferably by admixing the gold nanoparticles with the nucleic acid molecules.

The method described in the following is particularly suitable in order to provide conjugated gold nanoparticles as described before according to the first aspect of the present invention.

Prior to further specifications of particularly preferred embodiments of the method according to the present invention, relevant definitions of terms are given with respect to a better understanding.

The term “unconjugated” or “naked” gold nanoparticle means that the surface of the gold nanoparticles is substantially free of any molecular attachments, in particular organic resins or side products. According to a preferred embodiment of the present invention the naked and/or unconjugated gold nanoparticles comprise a gold surface, wherein the gold surface is to at least 90%, preferably at least 95%, even more preferred to at least 99% not attached to any molecules and freely accessible. In other words, on the basis of laser ablation, in particular pulsed laser ablation in liquid, ligand-free gold nanoparticles are synthesized.

The laser ablation, in particular the pulsed laser ablation in liquid, is known to the skilled practitioner, as already delineated with regard to the conjugated gold nanoparticles as such. The following settings of the laser ablation has been proven to be particularly advantageous with respect to the properties of the gold nanoparticles against the background of an improved therapeutic concept for the treatment of monogenetic disorders.

According to a preferred embodiment of the present invention, laser ablation is performed with a pulsed laser irradiation having a wave length in the range from 330 to 1,500 nm, preferably in the range from 800 to 1,200 nm.

Furthermore, according to another preferred embodiment of the present invention, the pulse energy is in the range of 1 to 1,000 μJ especially 5 to 500 μJ, particularly 10 to 250 μJ, preferably 50 to 200 μJ, even more preferred 90 to 150 μJ.

With respect to the pulse repetition rate it is advantageous when the pulse repetition rate is in the range of 1 to 1,000 kHz, especially 5 to 500 kHz, particularly 10 to 250 kHz, preferably 50 to 200 kHz, even more preferred 80 to 150 kHz.

Furthermore, it is advantageous when the pulse duration is in the range of 0.1 to 500 ps, especially 0.5 to 100 ps, particularly 1 to 50 ps, preferably 2 to 25 ps, even more preferred 5 to 15 ps.

On the basis of the aforementioned parameters, gold nanoparticles are produced, which are particularly suitable for the use in the medical field, in particular a non-viral gene therapy. In this context, it is of particular interest, that the gold nanoparticles are produced with an average particle diameter that allows the gold nanoparticles to be taken up by cells, in particular mammalian cells, preferably human cell types. Nevertheless, the particle size should not be linked with a higher cell toxicity.

Therefore, according to a preferred embodiment of the present invention, the the gold nanoparticles are adjusted to an average particle diameter dₚ [nm] in the range from 0.01 to 100 nm, in particular 0.05 to 80 nm, preferably 0.1 to 60 nm, particularly preferred 0.5 to 50 nm, even more preferred 1 to 25 nm, especially preferred 2 to 10 nm, preferably determined by analytical disc centrifugation (ADC) and/or transmission electron microscopy (TEM) and/or UV/VIS spectra. As delineated in connection with the conjugated gold nanoparticles as such, are particles with the aforementioned sizes able to be taken up by the cell and thereby still non-toxic.

The particle size, in particular the average particle diameter, is adjusted by variation of laser energy, wavelength of the pulsed laser irradiation, pulse duration, repetition rate and duration of laser ablation. The above-described parameters are particularly suitable in order to provide particles having the preferred sizes, which enable the gold nanoparticles to cross the membrane of the target cells without showing a significant toxicity or immunogenicity.

According to a preferred embodiment of the present invention, a gold target is used for laser ablation, wherein the gold nanoparticles are ablated from such gold target. In this context, it is particularly preferred when the gold target has a thickness in the range of 0.1 to 20,000 μm, especially 1 to 15,000 μm, particularly 10 to 10,000 μm, preferably 50 to 8,000 μm, even more preferred 100 to 5,000 μm. It is particularly preferred to use gold foil as gold target for laser ablation.

In order to provide a good compatibility of the gold nanoparticles, it is preferred when laser ablation is performed in a non-toxic, compatible liquid and/or medium. Therefore, according to a preferred embodiment of the present invention, laser ablation, in particular pulsed laser ablation in liquid, is performed in (i) purified water and/or (ii) phosphate based buffer, preferably sodium phosphate buffer (NaPB) and/or phosphate buffer saline (PBS) as liquid.

The conjugation of the gold nanoparticles with polyethylenimine as transfection reagent, i.e. method step (b) according to the present invention, can be performed in different ways, which are delineated in the following:

With respect to a first preferred embodiment of the present invention, method step (b) and/or conjugating the gold nanoparticles with polyethylenimine and/or derivatives and/or salts thereof is performed simultaneously with method step (a) and/or laser ablation of the unconjugated (naked) gold nanoparticles. In this context, the laser ablation, in particular the pulsed laser ablation in liquid, is performed in the presence of polyethylenimine and/or derivatives and/or salts thereof.

According to this preferred embodiment it was surprisingly found that a stable conjugation of the gold nanoparticles with polyethylenimine can be achieved on the basis of the addition of the transfection agent to the liquid used for laser ablation. In this context, it was particularly surprising that the laser pulses are not hindering or interfering with respect to the interaction between the gold nanoparticles and the transfection agent. For, the bonding of the transfection reagent to the gold nanoparticles is based on rather weak electrostatic interactions on the basis—without being bound to this theory—of the partial charges of gold, on the one hand, and the nitrogen atoms of the transfection agent, on the other hand. Despite the high energy input by the laser, a sufficient conjugation of the gold nanoparticles with the transfection reagent is achieved according to this embodiment of the method.

In order to achieve a good loadability of the conjugated gold nanoparticles with nucleic acid molecules and to provide an efficient gene transfer and uptake of the particles by the cells, it is particularly preferred when polyethylenimine and/or derivatives and/or salts thereof is added to the liquid, especially wherein polyethylenimine and/or derivatives and/or salts thereof is added to a concentration in the range from 0.1 to 1.000 μg/ml, especially in the range from 0.5 to 800 μg/ml, preferably in the range from 5 to 500 μg/ml, in particular in the range from 10 to 300 μg/ml, particularly preferred in the range from 20 to 200 μg/ml, based on the liquid for pulsed laser ablation.

According to a second, likewise preferred embodiment of the present invention, conjugating the gold nanoparticles with the transfection agent polyethylenimine is performed after generating the unconjugated, naked gold nanoparticles by laser ablation:

According to this further preferred embodiment of the present invention, method step (b) and/or conjugating the gold nanoparticles with polyethylenimine and/or derivatives and/or salts thereof is performed by admixing the laser-ablated gold nanoparticles with polyethylenimine and/or derivatives and/or salts thereof. In particular, according to this embodiment of the present invention admixing the gold nanoparticles with polyethylenimine and/or derivatives and/or salts thereof is performed as a separate method step and/or simultaneously with method step (c), i.e. the conjugation of the gold nanoparticles with nucleic acid molecules.

Both embodiments of the present invention with respect to conjugating the gold nanoparticles with the transfection reagent lead to highly competent conjugated gold nanoparticles with a high loadability for nucleic acid molecules and a high transfection efficiency, in particular an improved ability to cross the membrane of the target cells with subsequent endosomal release of the nucleic acid molecules.

With respect to method step (c) and/or conjugating the gold nanoparticles with nucleic acid molecules, admixing the nucleic acid molecules with the nanoparticles can be performed immediately before and/or within tranfection.

According to the present invention it was surprisingly found that gold nanoparticles obtained by laser ablation, in particular pulsed laser ablation in liquid, provide a particularly good loadability with respect to the transfection agent and the nucleic acid molecules.

Particularly good results with respect to the transfer of genetic material as well as the transfection efficiency are achieved when the conjugated gold nanoparticles prepared by the method of the present invention comprise the nanoparticles and the transfection agent in a defined weight related ratio. In order to provide such gold nanoparticles with improved properties, it is preferred when polyethylenimine and/or derivatives and/or salts thereof and gold nanoparticles are employed in the method of the present invention in a weight related ratio in the range from 1:100 to 100:1, especially from 1:50 to 50:1, preferably from 1:30 to 20:1, in particular preferred from 1:20 to 10:1, even more preferred from 1:10 to 1:1.

In this context, with respect to an efficient load of the conjugated gold nanoparticles with nucleic acid molecules, it is also preferred when polyethylenimine and/or derivatives and/or salts thereof and nucleic acid molecules are employed in a weight related ratio of polyethylenimine and/or derivatives and/or salts thereof to nucleic acid molecules in the range from 1:100 to 150:1, especially from 1:50 to 100:1, preferably from 1:20 to 50:1, in particular preferred from, 1:10 to 20:1, even more preferred from 1:1 to 10:1.

Overall, the high loadability of the laser-ablated gold nanoparticles with polyethylenimine, on the one hand, and nucleic acid molecules, on the other hand, was completely surprising and not foreseeable at all. Particularly good results with respect to transfection efficiency and gene transfer efficiency are achieved, when transfection agents, gold nanoparticles and nucleic acid molecules are employed in the above describe weight related ratios.

According to a particularly preferred embodiment of the present invention, the method for preparation of gold nanoparticles is suitable to provide conjugated gold nanoparticles comprising a so called layer-by-layer assembly on the basis of alternating layers of polyethylenimine and nucleic acid molecules. In order to provide such layer-by-layer assembly, subsequent to method steps (a) to (c) a method step further method step (d) is performed, wherein in method step (d) the particles obtained by method steps (a) to (c) are conjugated with a further outer layer comprising polyethylenimine and/or derivatives and/or salts thereof, preferably galactose-conjugated polyethylenimine and/or derivatives and/or salts thereof.

As described above in connection with the conjugated gold nanoparticles as such, a layer-by-layer assembly is advantageous with respect to an increase of the transfection efficiency. Furthermore, on the basis of galactose-conjugated polyethylenimine in the outer layer, conjugated gold nanoparticles allowing a purposeful targeting of the transfection of the target cells, in particular liver cells, can be prepared.

Overall, the present invention does not only provide conjugated gold nanoparticles as such, but also a method which is suitable to obtain such particles.

For further details concerning this aspect of the invention, reference can also be made to the above explanations with respect to the aspects outlined before, said explanations also applying accordingly with regard to the method according to the present invention.

Furthermore, subject-matter of the present invention—according to a fourth aspect of the present invention—is a nanoparticle-based delivery system for a coding sequence, preferably for the use in the treatment, in particular non-viral gene therapy, of a monogenetic disorder resulting from a mutation in a gene coding for a liver-specific and/or liver-expressed protein, wherein the delivery system comprises conjugated gold nanoparticles as described above according to the first aspect of the present invention and a physiologically and/or pharmaceutically acceptable carrier.

According to a preferred embodiment, the nanoparticle-based delivery system is prepared as a medicament, drug, pharmaceutical drug and/or agent, i.e. the nanoparticle-based delivery system is prepared as a drug used to diagnose, cure, treat or prevent diseases, in particular monogenetic disorders, as described before.

With respect to a particular preferred embodiment of the present invention, the nanoparticle-based delivery system is prepared for a systemic application, in particular an intravenous and/or oral, preferably systemic application.

With respect to the use of the nanoparticle-based delivery system according to the present invention it is preferred when the disorder or disease to be treated is associated with an impaired and/or reduced hemostasis and/or blood clotting, especially wherein the disorder is a hemophilia, in particular hemophilia A and/or hemophilia B.

For further information with respect to this aspect of the present invention, reference can also be made to the afore described aspects, wherein said explanations with respect to the aforementioned aspects also apply accordingly with respect to this aspect of the present invention.

Also subject-matter of the present invention is—according to a fifth aspect of the present invention—the use of a delivery system as described before in the treatment, in particular a non-viral gene therapy, of a monogenetic disorder resulting from a mutation in a gene coding for a liver-specific and/or liver-expressed protein and/or for the preparation of a medicament for the treatment of a monogenetic disorder resulting from a mutation in a gene coding for a liver-specific and/or liver-expressed protein.

In this context, it is particularly preferred when monogenetic disorder is associated with an impaired and/or reduced hemostasis and/or blood clotting, especially wherein the disorder is a hemophilia, in particular hemophilia A and/or hemophilia B.

For further details concerning this aspect of the present invention, reference can be made to the above explanations in relation to the aspects outlined before, said explanations also applying accordingly with regard to this aspect of the present invention.

Additionally, subject-matter of the present invention is—according to a sixth aspect of the present invention—a method for the transfection of target cells, especially mammalian cells, preferably human cells, preferably liver-cells and/or fibroblasts, wherein conjugated gold nanoparticles as described before are used in that method.

With respect to the method for transfection of target cells, reference is made to the above-described aspects of the present invention as well as the working examples.

For further details concerning this aspect of the present invention, reference can be made to the above explanations in relation to the aspects outlined before, said explanations also applying accordingly with regard to this aspect of the present invention.

Furthermore, subject-matter of the present invention is—according to a seventh aspect of the present invention—a transfected cell, preferably mammalian, in particular human cell, especially for the use in the treatment, in particular non-viral gene therapy, of a monogenetic disorder resulting from a mutation in a gene coding for a liver-specific and/or liver-expressed protein, wherein transfection has been performed with conjugated gold nanoparticles as described above and/or wherein the transfected cell comprises conjugated gold nanoparticles as described above.

With respect to the transfected cells, reference is made to the above-described aspects of the present invention as well as the working examples.

For further details concerning this aspect of the present invention, reference can be made to the above explanations in relation to the aspects outlined before, said explanations also applying accordingly with regard to this aspect of the present invention.

Finally, subject-matter of the present invention is—according to an eighth aspect of the present invention—a vector, in particular non-viral vector, preferably for the expression of a liver-specific and/or liver-expressed protein and/or preferably physiologically active domains and/or fragments thereof in a patient suffering from a monogenetic disorder caused by a mutation in the gene coding for the liver-specific and/or liver-expressed protein, wherein the vector comprises:
(a) a promoter, wherein the promoter is derived from a human gene;
(b) a coding sequence containing a nucleic acid sequence coding for a liver-specific and/or liver-expressed protein and/or preferably physiologically active domains and/or fragments thereof, wherein mutations in the nucleic acid sequence coding for the liver-specific and/or liver-expressed protein are associated with a monogenetic disorder;
(c) a nucleic acid sequence derived from the scaffold/matrix attachment region of a eukaryotic, preferably human gene; and
(d) a transcriptional termination signal.

The vector according to the present invention is particularly suitable for the use in conjugated gold nanoparticles according to the present invention. In particular, the vector allows an expression of the coding sequence in the transfected target cells, preferably in order to compensate an impaired or total loss of the endogenous production of the respective liver-specific and/or liver-expressed protein.

With respect to the elements of the vector, in particular the coding sequence, the scaffold/matrix attachment region and the promoter, reference can be made to above explanations with respect to the nucleic acid sequences, in particular the vector, used in the conjugated gold nanoparticles according to the first aspect of the present invention.

However, according to a particularly preferred embodiment of the present invention, the promoter is derived from the gene coding to human Elongation Factor-1 alpha (EF1a) and/or from the human SERPINA1 promoter and/or from the hAAT (human 1-antitrypsin) promoter.

With respect to the promoter, it is particularly preferred when the promoter comprises a nucleotide sequence according to SEQ ID NO. 3, SEQ ID NO. 4 and/or SEQ ID NO. 5, especially SEQ ID NO. 3 and/or SEQ ID NO. 4. Likewise, the promoter can comprise a nucleic acid sequence having at least 85%, in particular at least 90%, preferably at least 95% identity with SEQ ID NO. 3, SEQ ID NO. 4 and/or SEQ ID NO. 5, especially SEQ ID NO. 3 and/or SEQ ID NO. 4.

Furthermore, it is also possible that the vector contains at least one further cis-regulatory element, especially at least one further transcriptional enhancer. In this context, it is particularly preferred when the cis-regulatory element is derived from the apolipoprotein E gene, in particular the apolipoprotein E hepatic locus control region (HCR). According to a preferred embodiment of the present invention, the cis-regulatory element has a nucleotide sequence according to SEQ ID NO. 6. Likewise, the cis-regulatory element can have a nucleic acid sequence having at least 85%, in particular at least 90%, preferably at least 95% identity with SEQ ID NO. 6.

For further details concerning this aspect of the present invention, reference can be made to the above explanations in relation to the aspects outlined before, said explanations also applying accordingly with regard to this aspect of the present invention.

Further advantages, properties and features of the present invention are apparent from the following description of preferred examples of the present invention shown in the drawings:

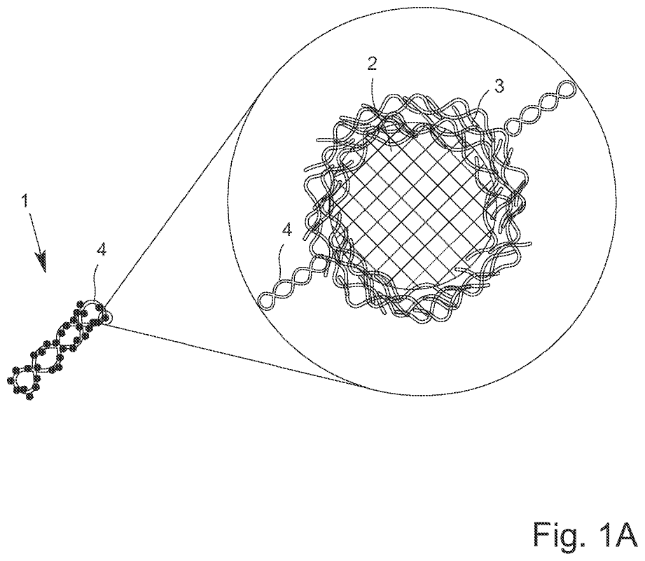

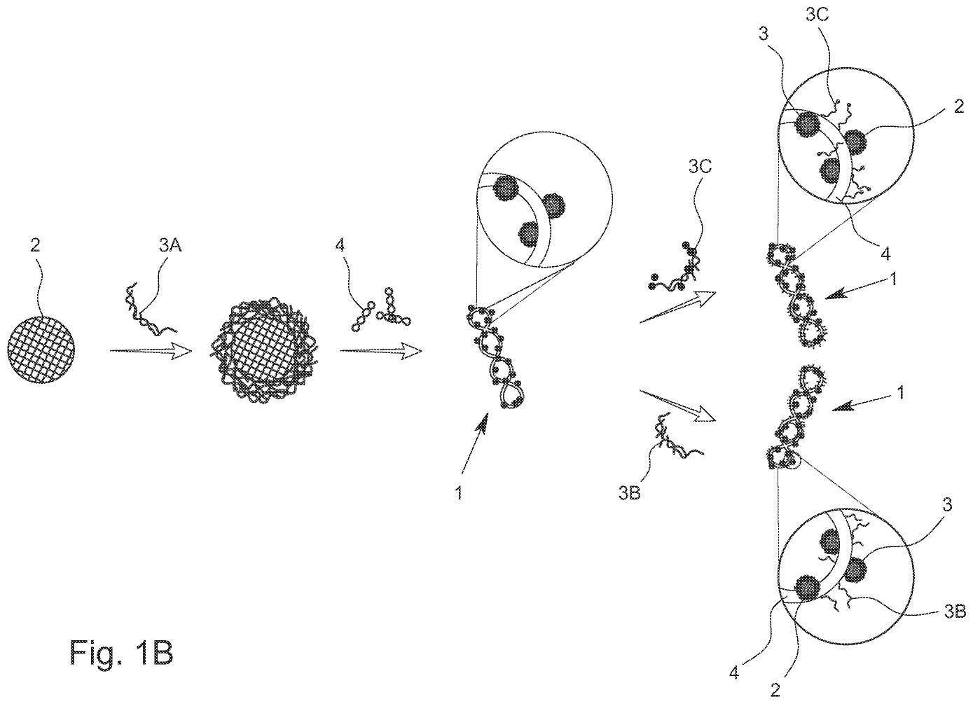

FIGS. 1A-1B shows a schematic representation of preferred embodiments of conjugated gold nanoparticles according to the present invention;

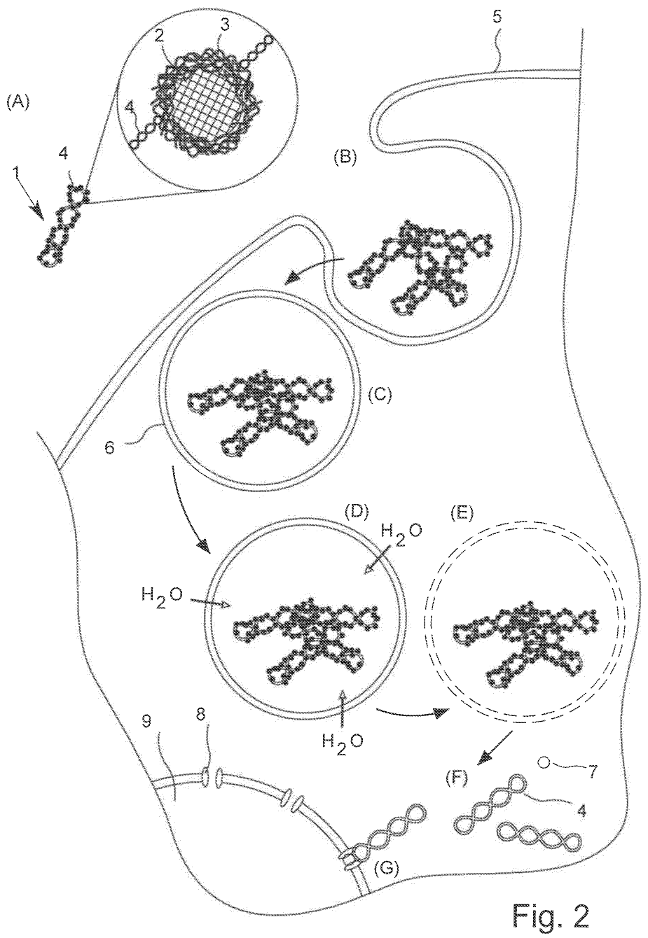

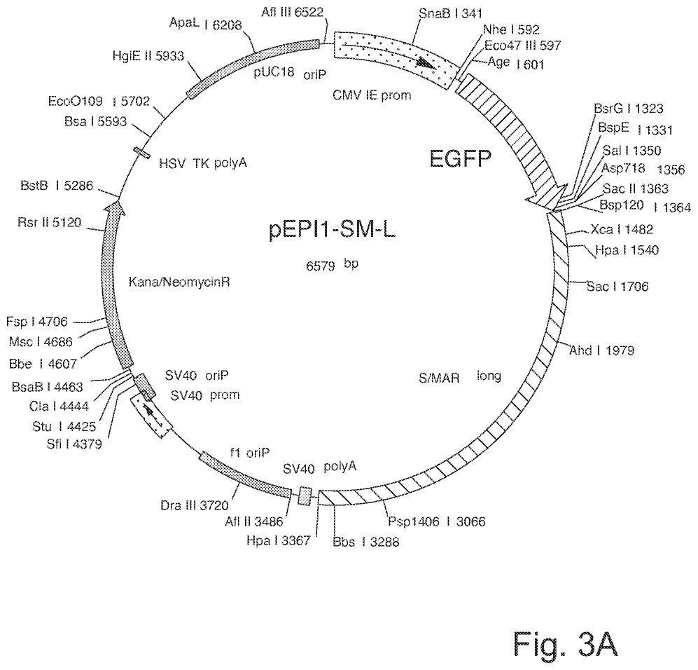

FIG. 2 shows a schematic representation of the transfection mechanism for the transfer of nucleic acid molecules into target cells on the basis of schematic illustrations of a section of a target cell during transfection with the conjugated gold nanoparticles according to the present invention;

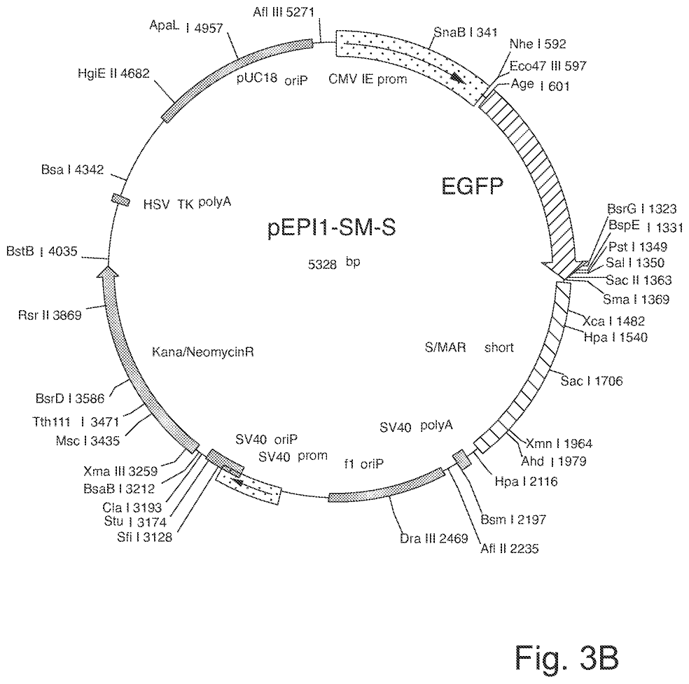

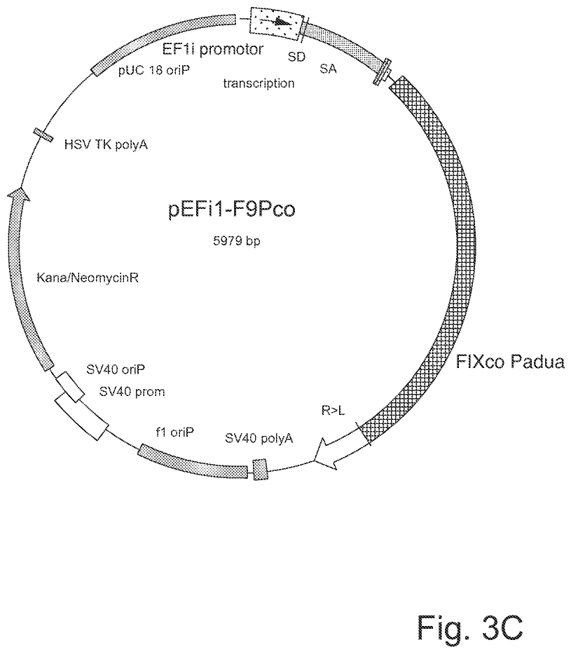

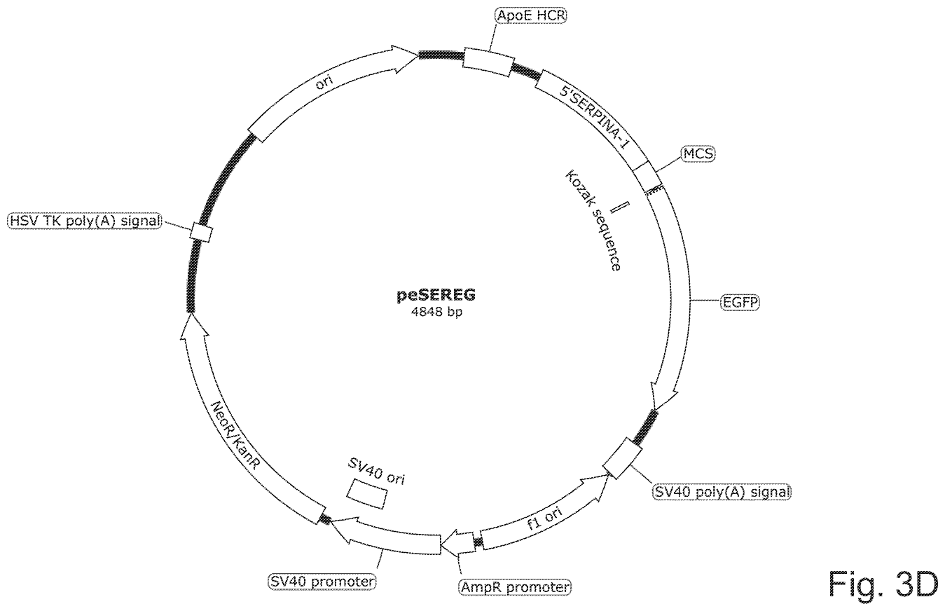

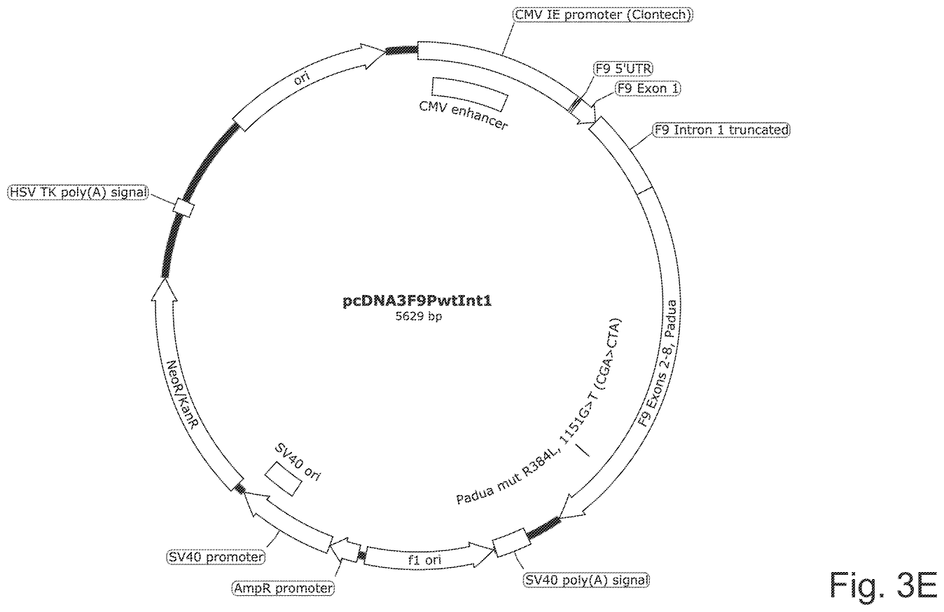

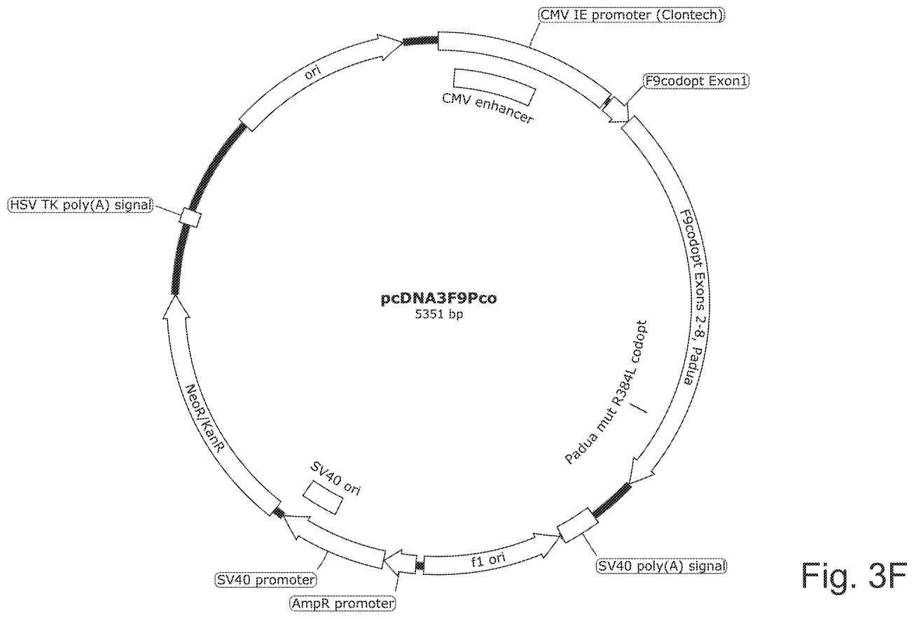

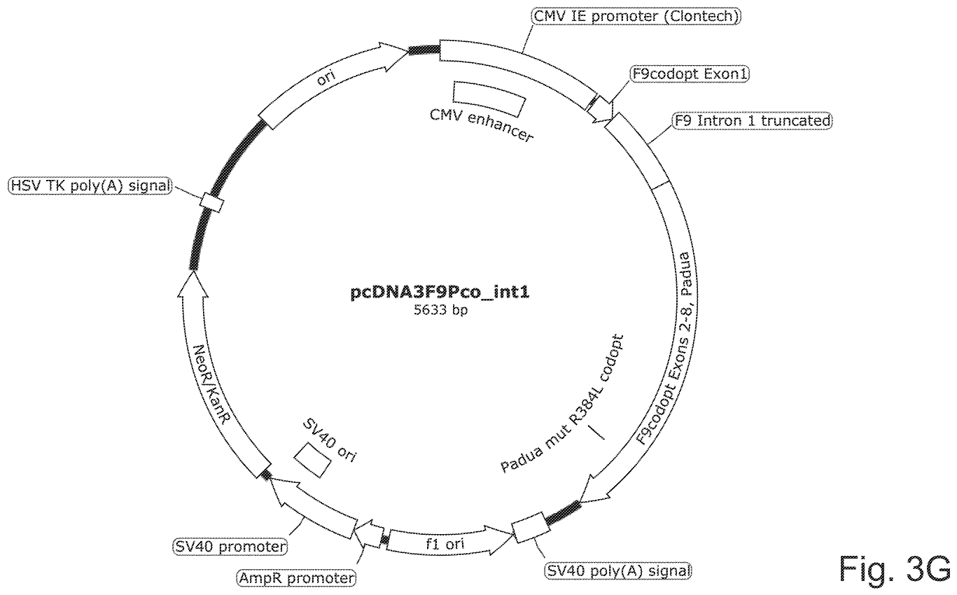

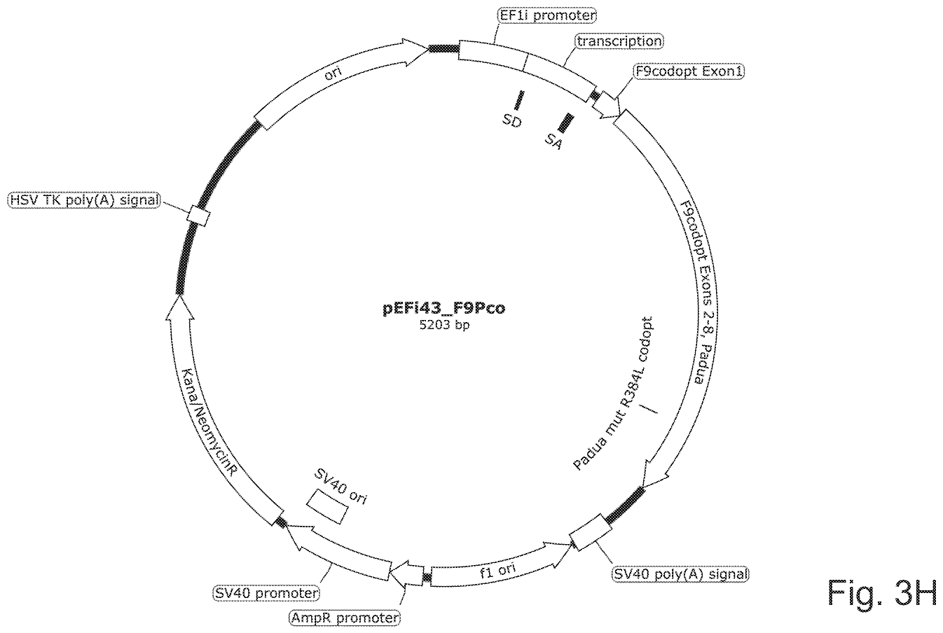

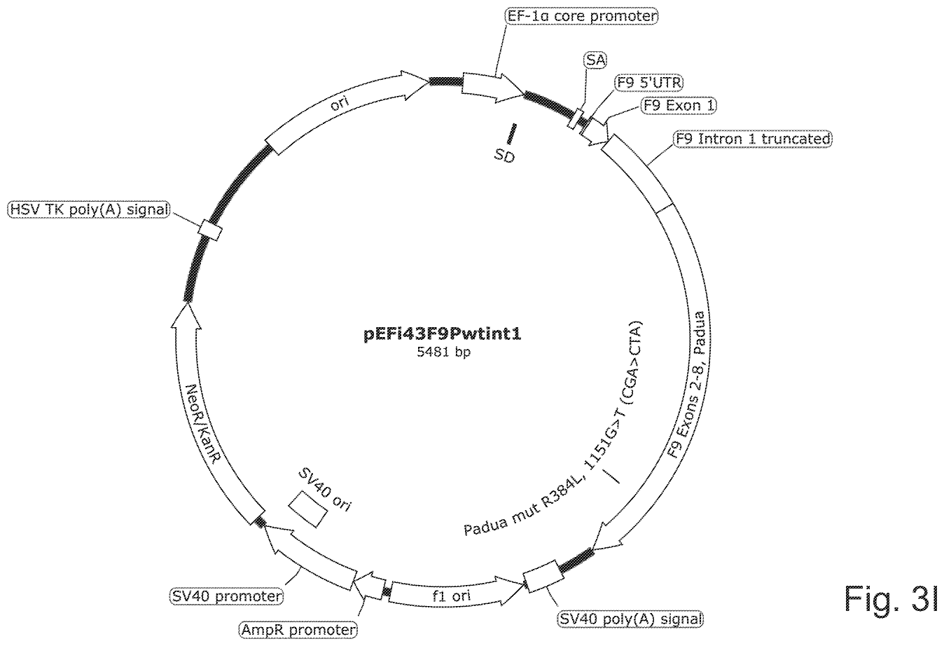

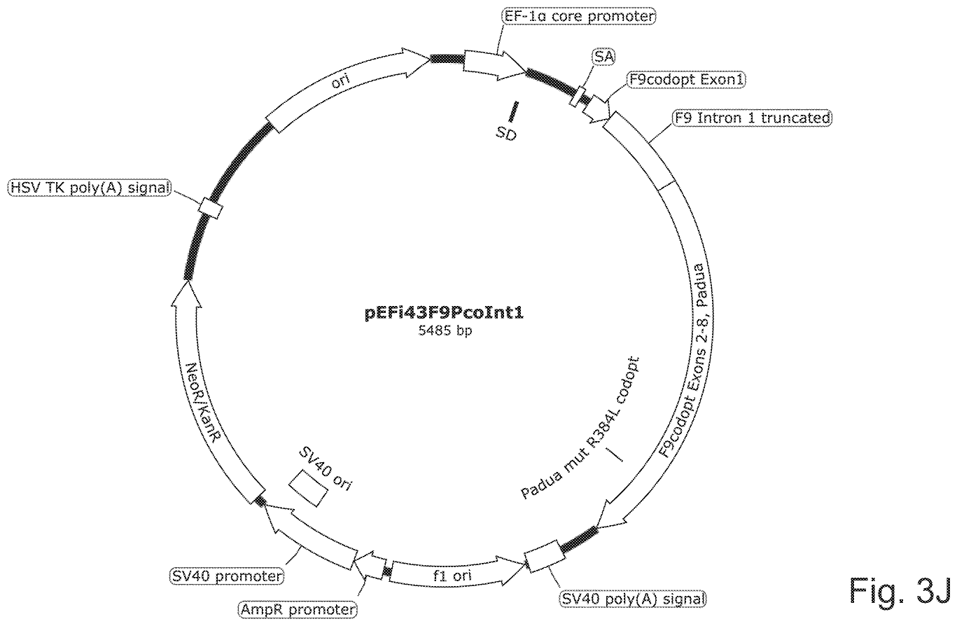

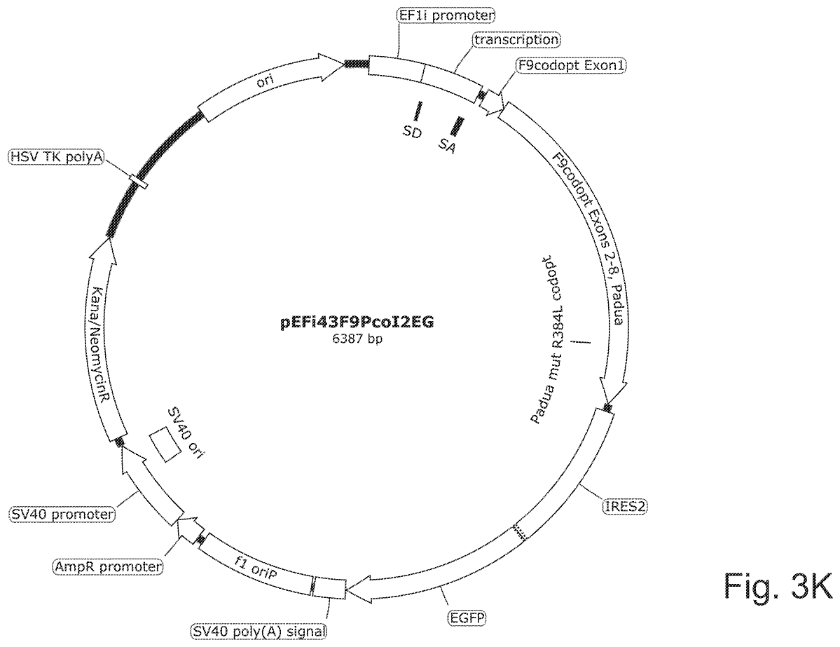

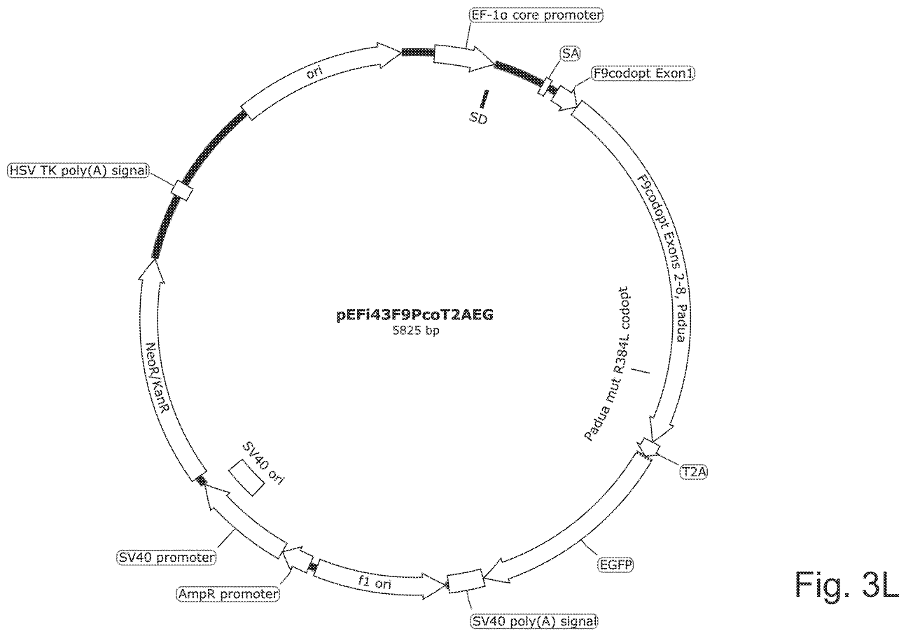

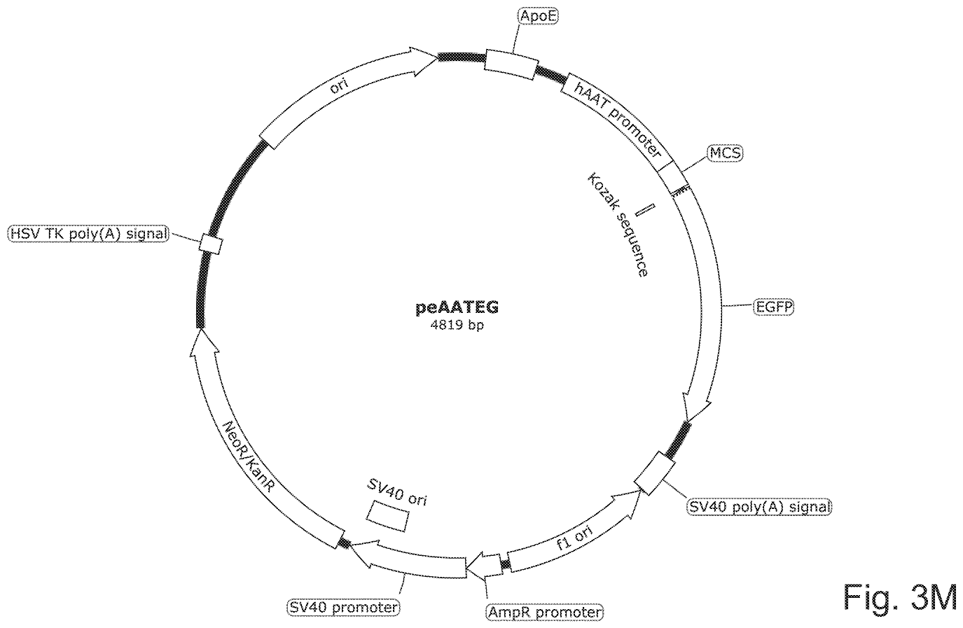

FIGS. 3A-3M shows schematic representations of plasmids and/or vectors, respectively, used for transfection experiments and studies in order to analyze the transfection efficiency of conjugated gold nanoparticles according to the present invention;

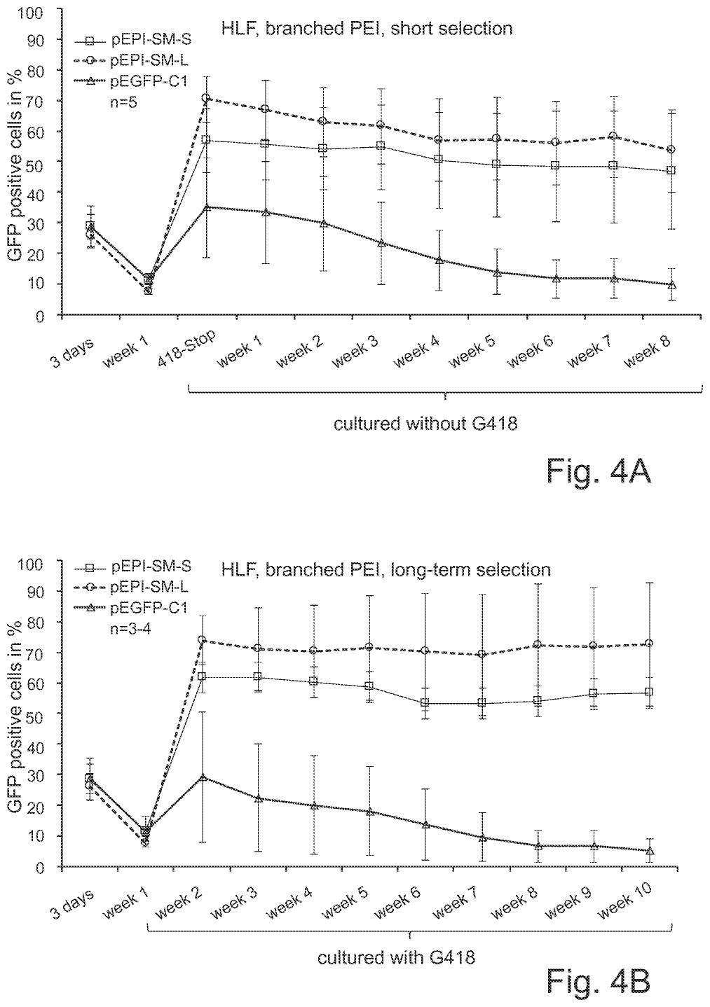

FIGS. 4A-4B shows the graphic representation of the result of studies in liver cancer cell line HLF concerning the effect of the presence of different S/MAR elements on the long-term expression levels of eGFP in transfected cells;

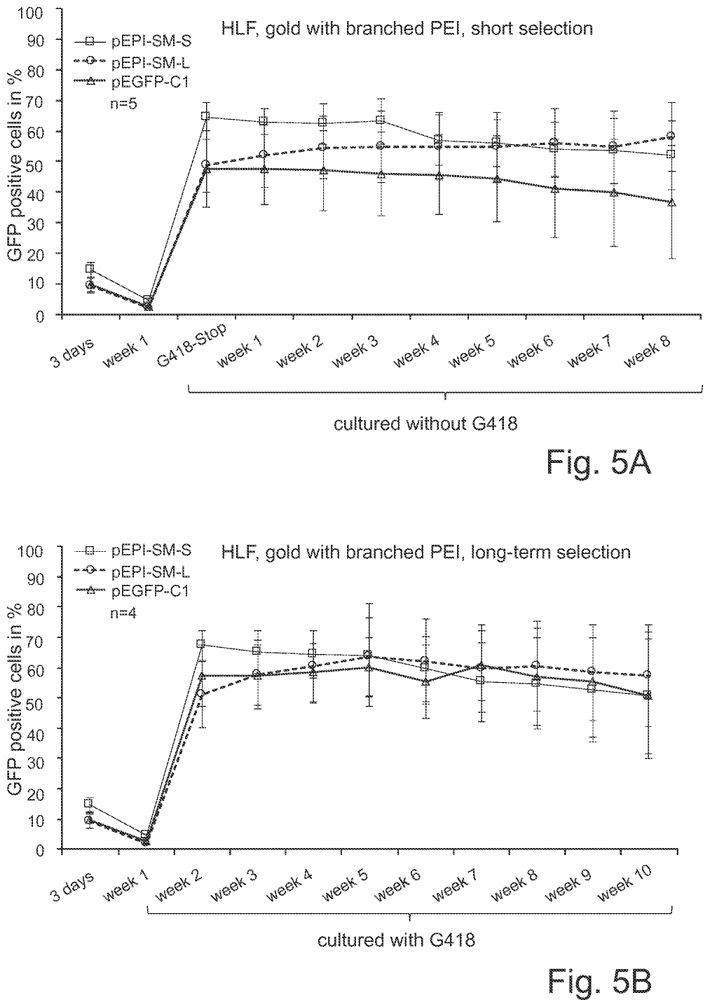

FIGS. 5A-5B shows the graphic representation of the result of studies in liver cancer cell line HLF concerning the effect of the presence of S/MAR elements on the long-term expression levels of eGFP in transfected cells, wherein conjugated gold nanoparticles according to the present invention were used for transfection;

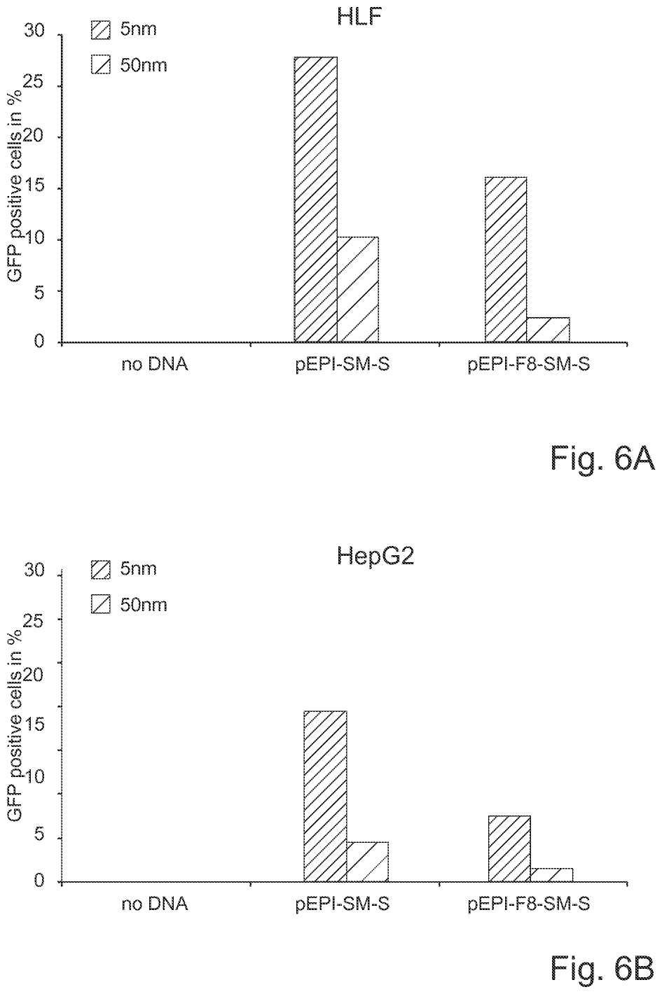

FIGS. 6A-6B shows a graphic representation of the results of studies in liver cell lines HLF and HepG2, wherein the influence of the particle size of the gold nanoparticles on transfection efficiency has been analyzed;

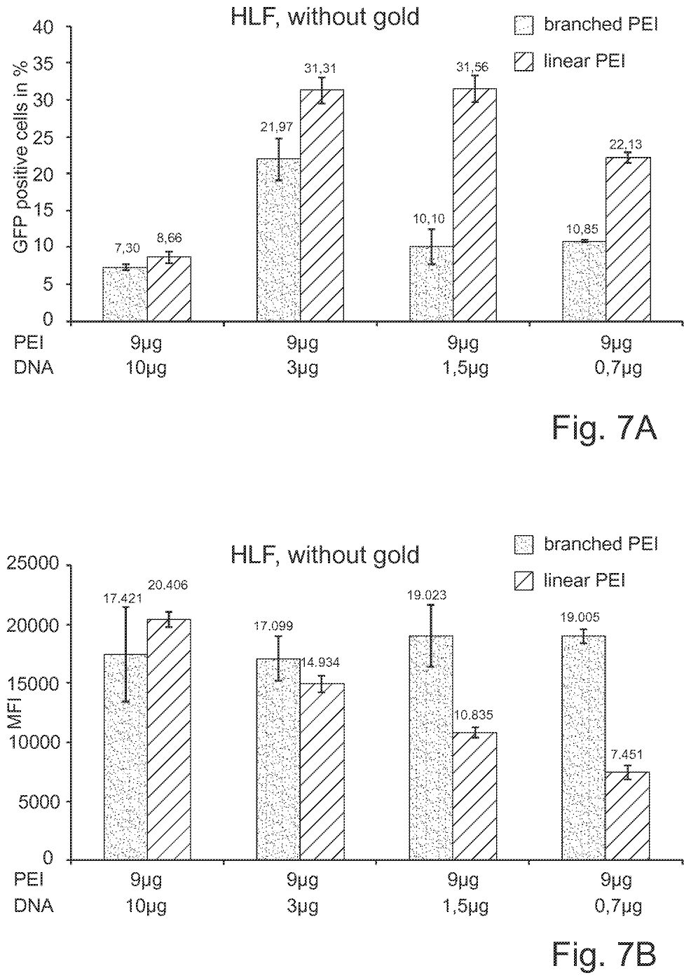

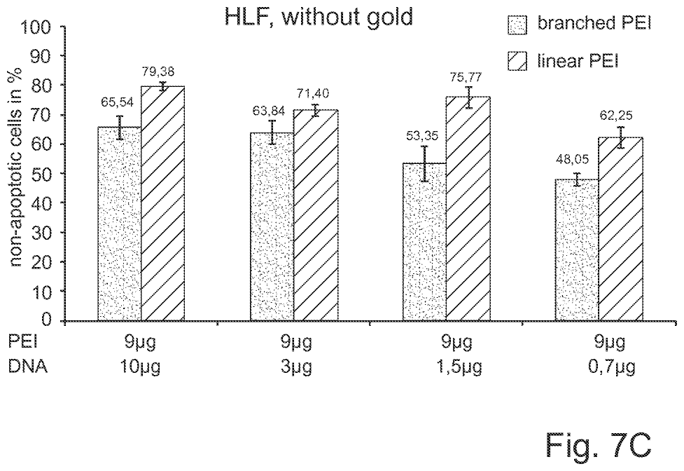

FIGS. 7A-7C shows the graphic representation of the results of studies in liver cancer cell line HLF, wherein the impact of the weight related ratio of nucleic acid molecules to polyethylenimine on the transfection efficiency has been analyzed;

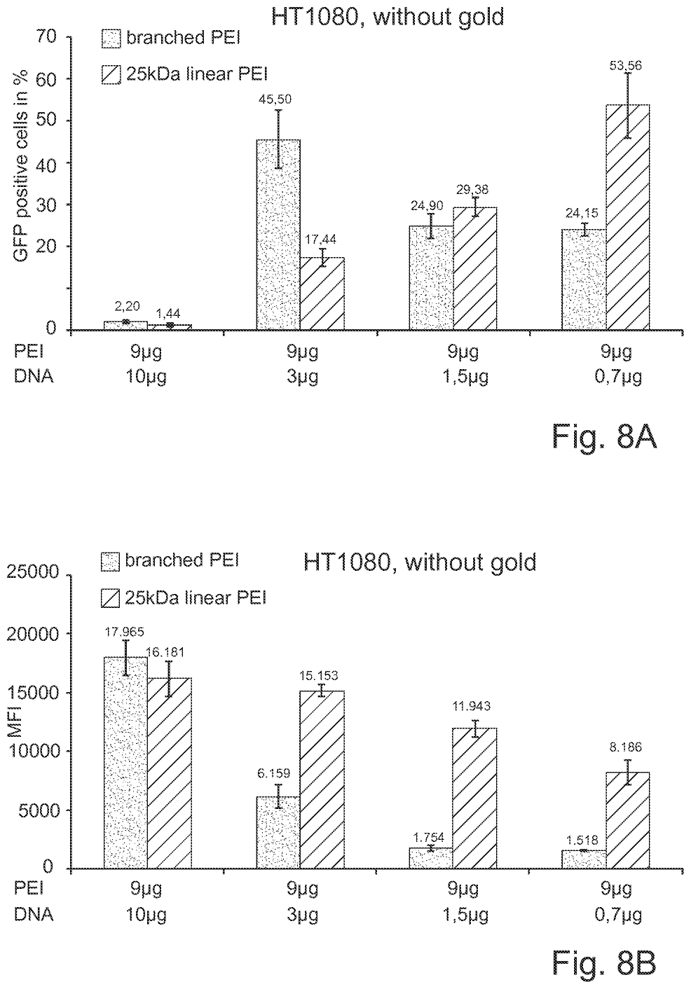

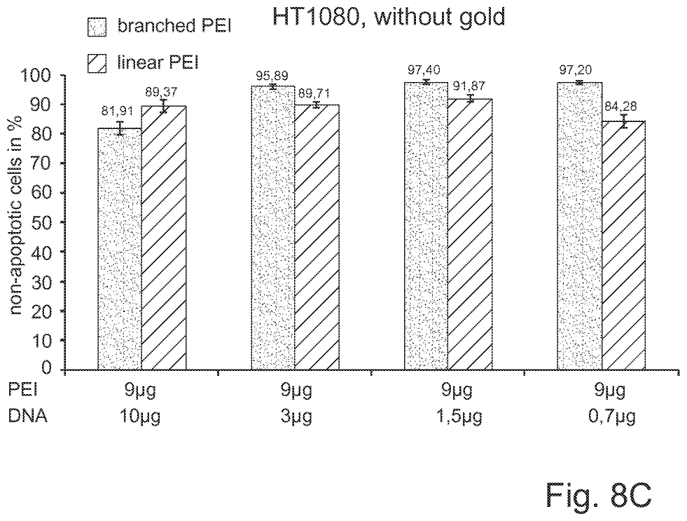

FIGS. 8A-8C shows the graphic representation of the results of studies in fibrosarcoma cell line HT1080, wherein the influence of the weight related ratio of DNA to polyethylenimine on the transfection efficiency has been analyzed;

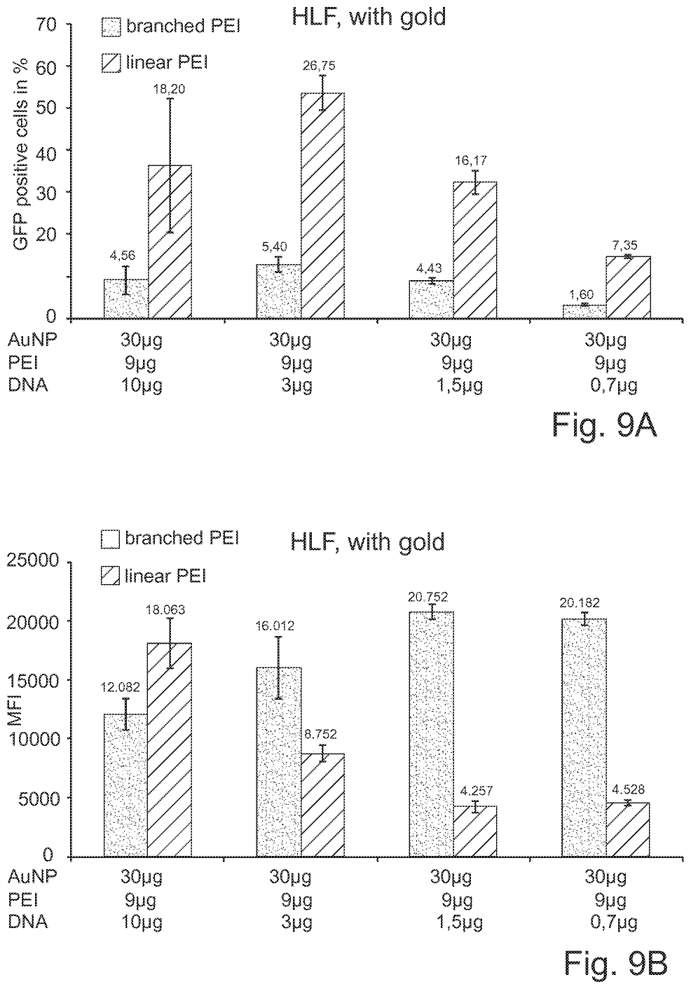

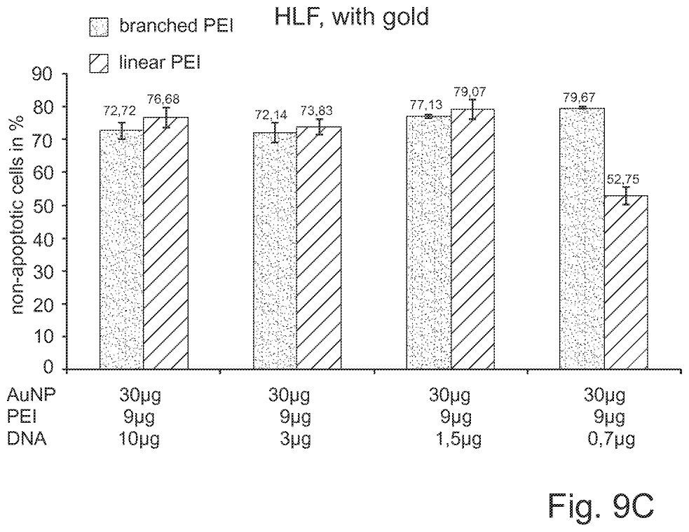

FIGS. 9A-9C shows the graphic representation of the result of studies in liver cancer cell line HLF, wherein the influence of the weight related ratio of nucleic acid molecules to transfection agent in conjugated gold nanoparticles has been analyzed.

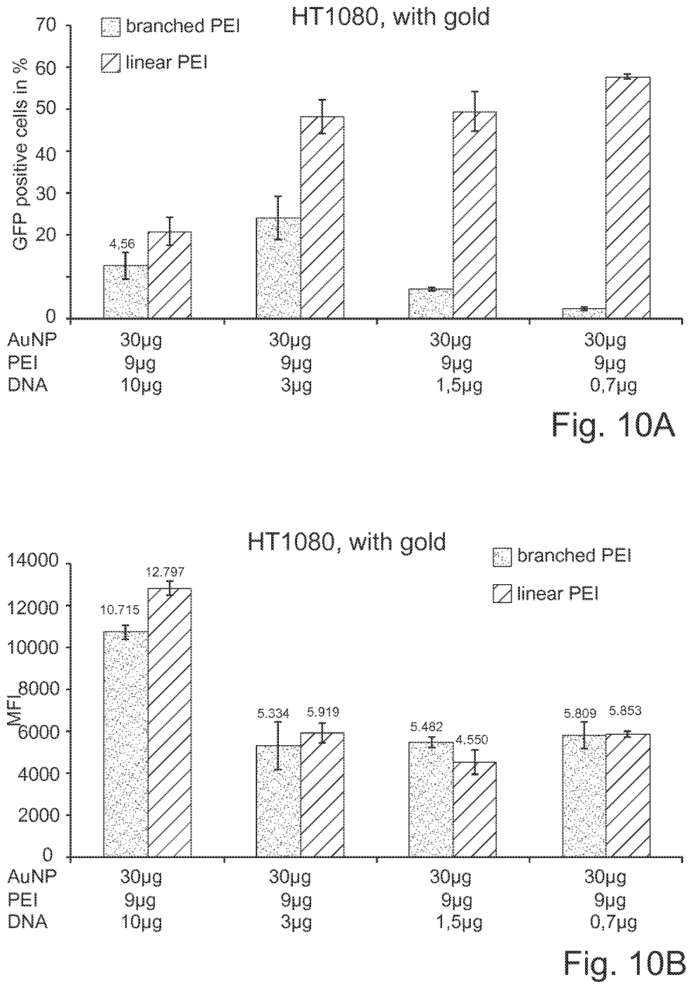

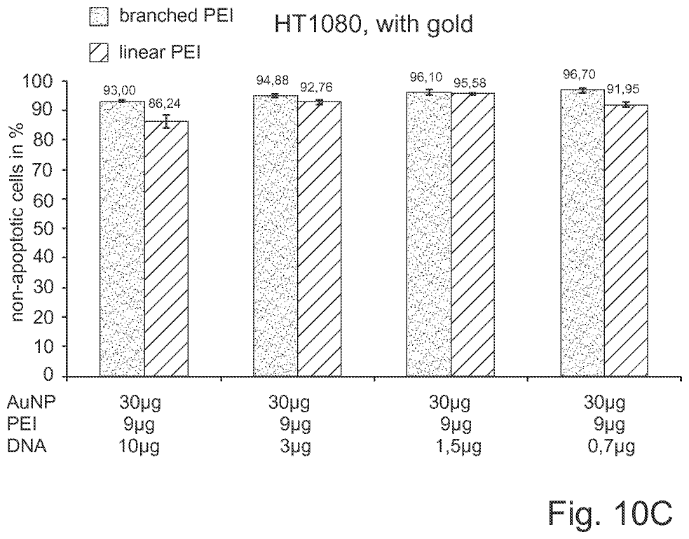

FIGS. 10A-10C shows the graphic representation of the results of studies performed in fibrosarcoma cell line HT1080, wherein the influence of the weight related ratio of polyethylenimine to nucleic acid molecules in conjugated gold nanoparticles has been analyzed;

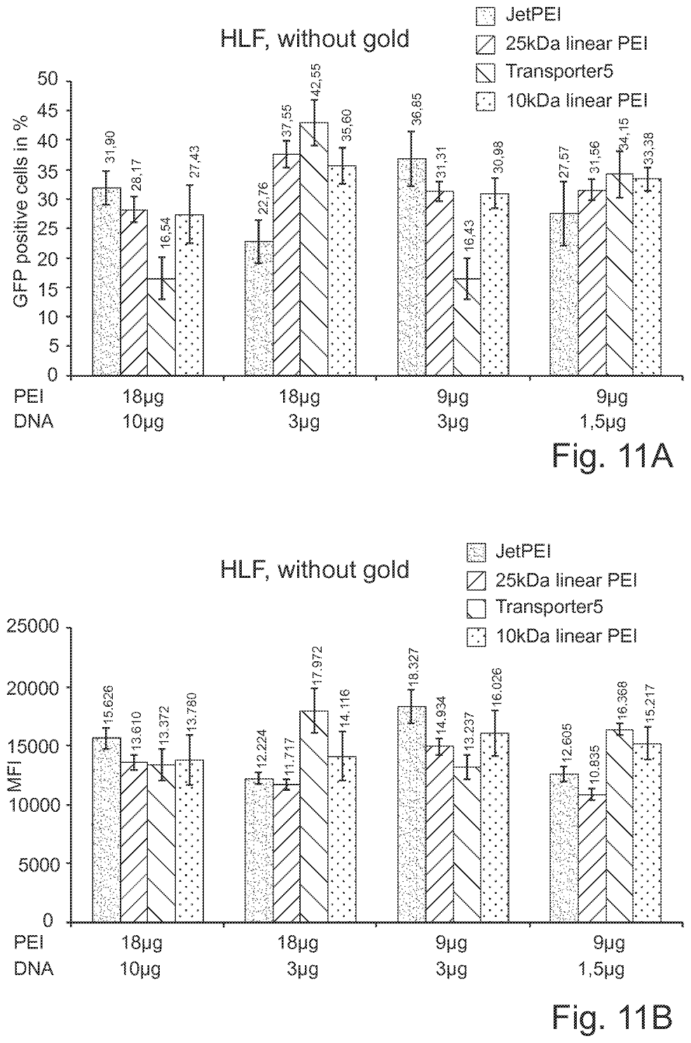

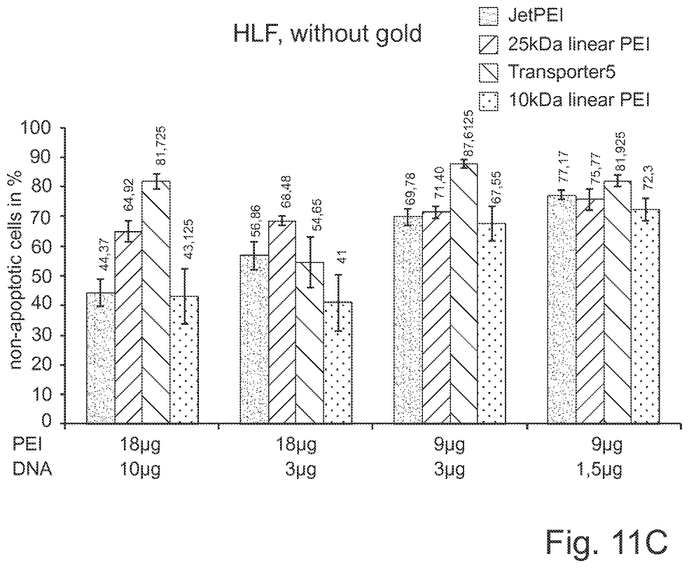

FIGS. 11A-11C shows the graphic representation of the results of studies in HLF cells, wherein the influence of different linear PEI variants on transfection efficiency and toxicity has been analyzed;

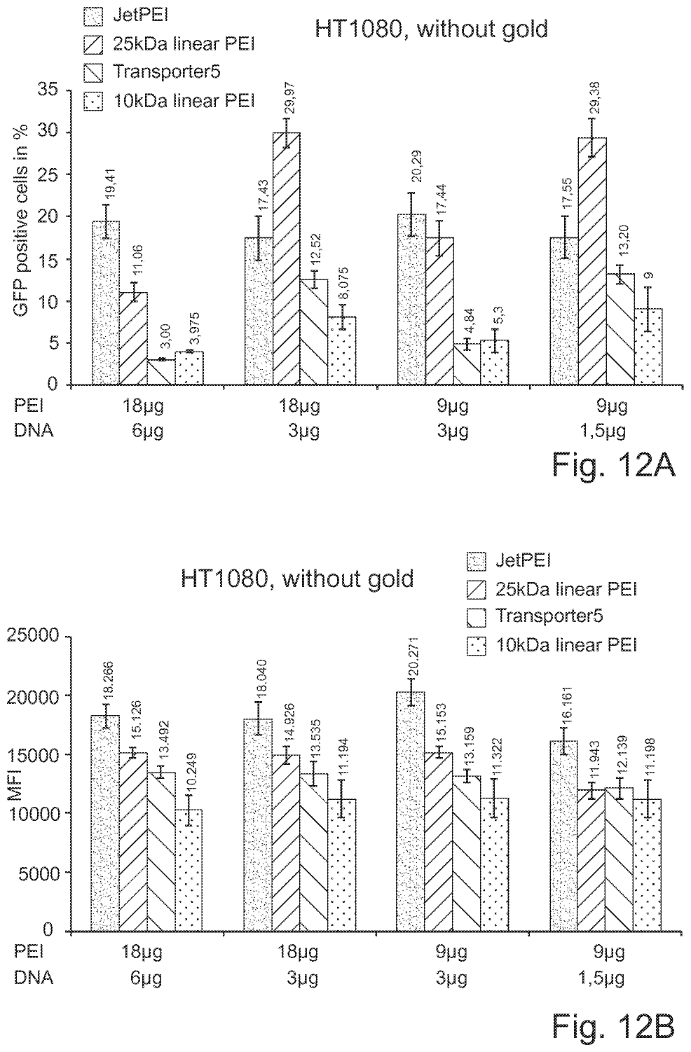

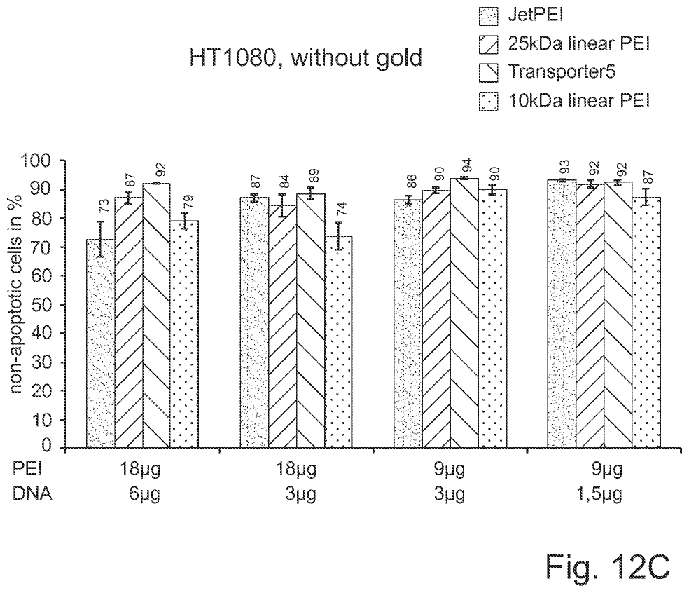

FIGS. 12A-12C shows the graphic representation of the results of studies in fibrosarcoma cells HT1080, wherein the influence of different linear PEI variants on transfection efficiency and toxicity has been analyzed;

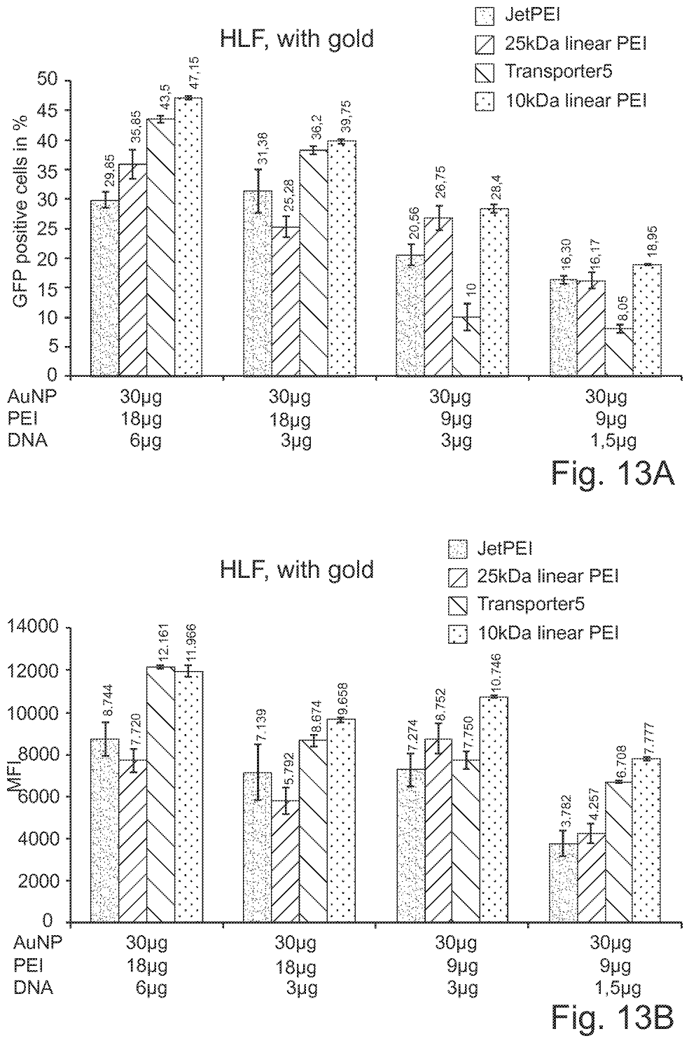

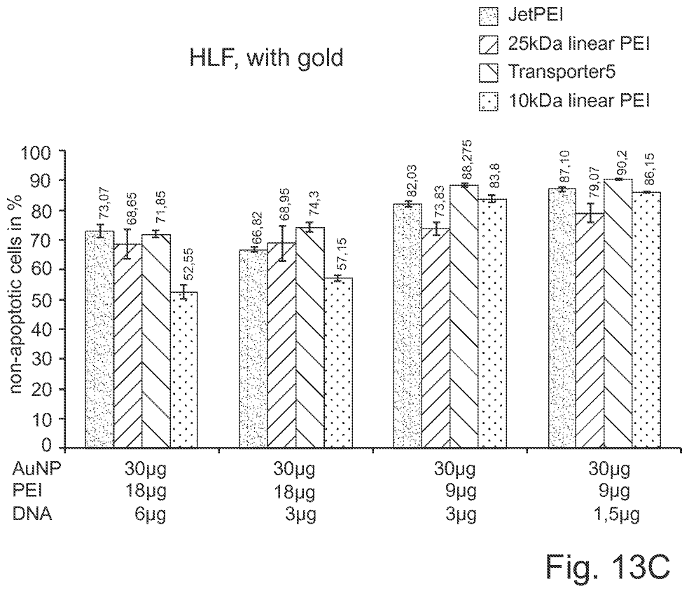

FIGS. 13A-13C shows the graphic representation of the results of studies in liver cancer cell line HLF, wherein the transfection efficiency of conjugated gold nanoparticles comprising different quantities of polyethylenimine and different weight related ratios of polyethylenimine to nucleic acid molecules has been analyzed;

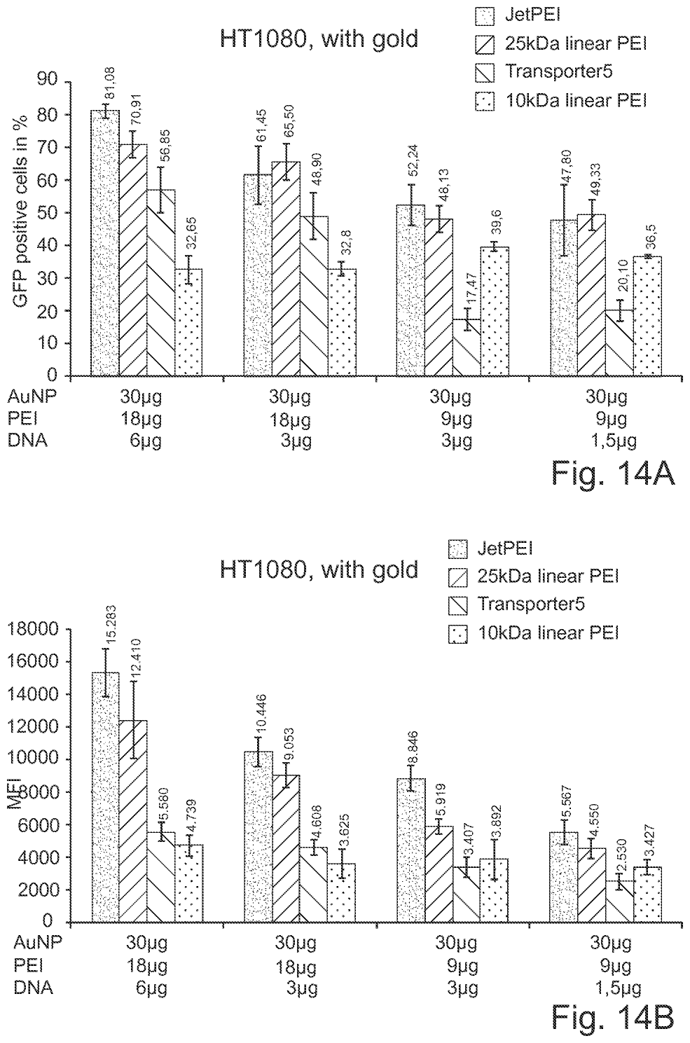

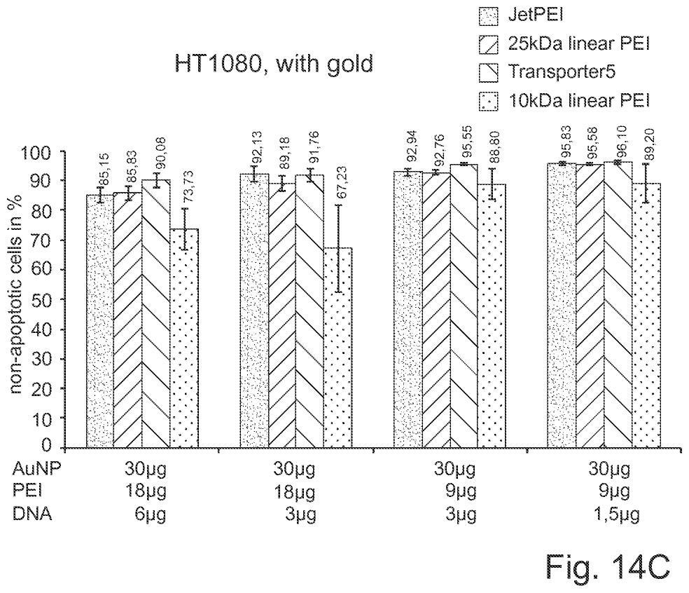

FIGS. 14A-14C shows the graphic representation of the results of studies performed in fibrosarcoma cell line HT1080, wherein the transfection efficiency of conjugated gold nanoparticles with different quantities of polyethylenimine and different weight related ratios of polyethylenimine to nucleic acid molecules has been analyzed;

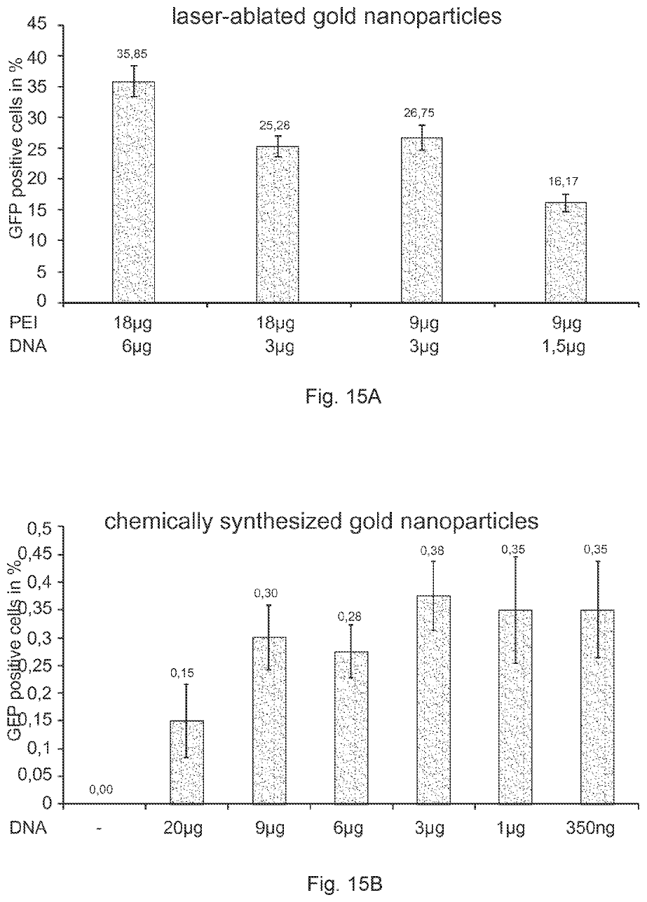

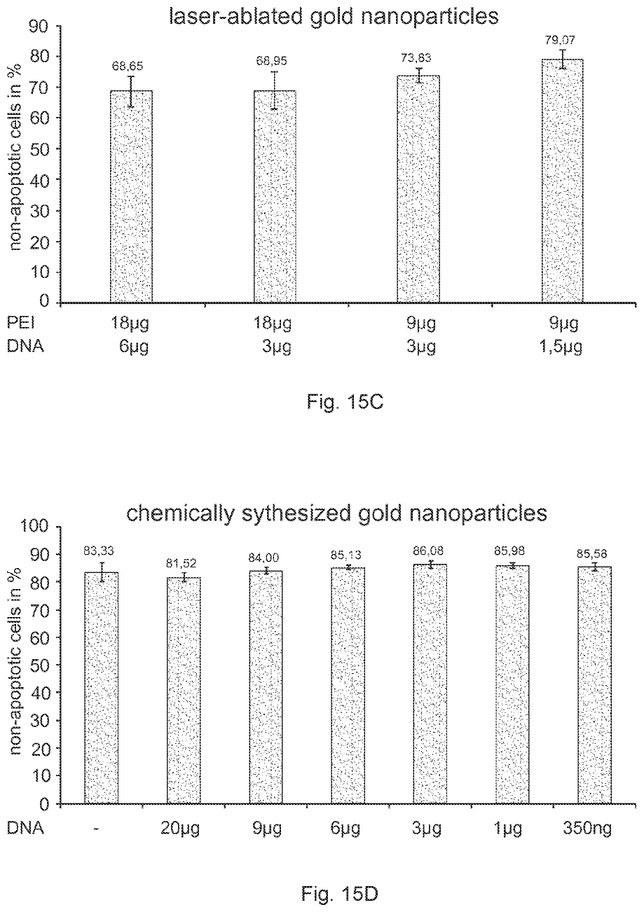

FIGS. 15A-15D shows the graphic representation of the results of studies in liver cancer cell line HLF, wherein conjugated gold nanoparticles obtained by laser ablation have been compared with comparative conjugated gold nanoparticles on the basis of chemically synthesized particles;

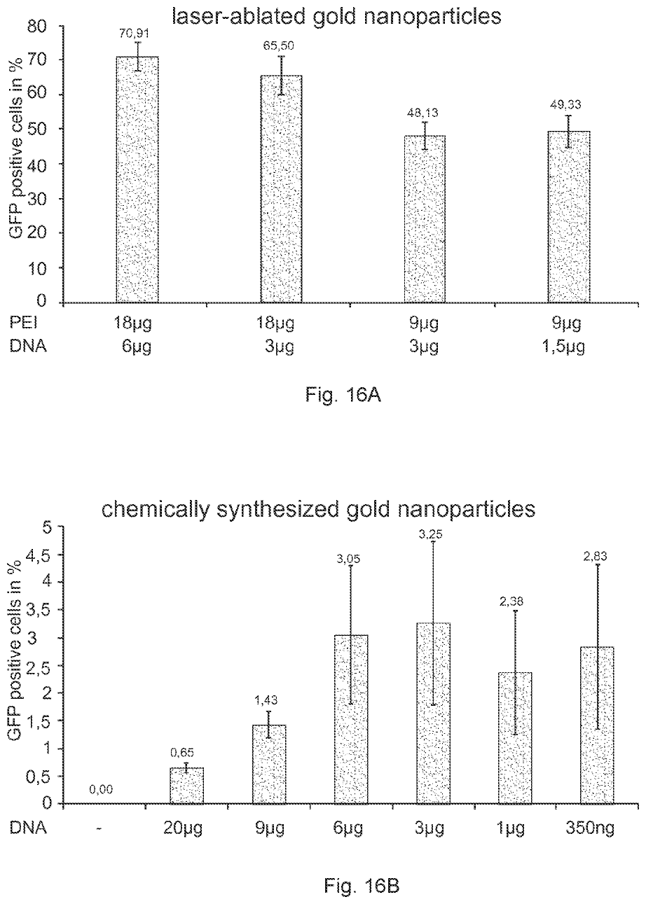

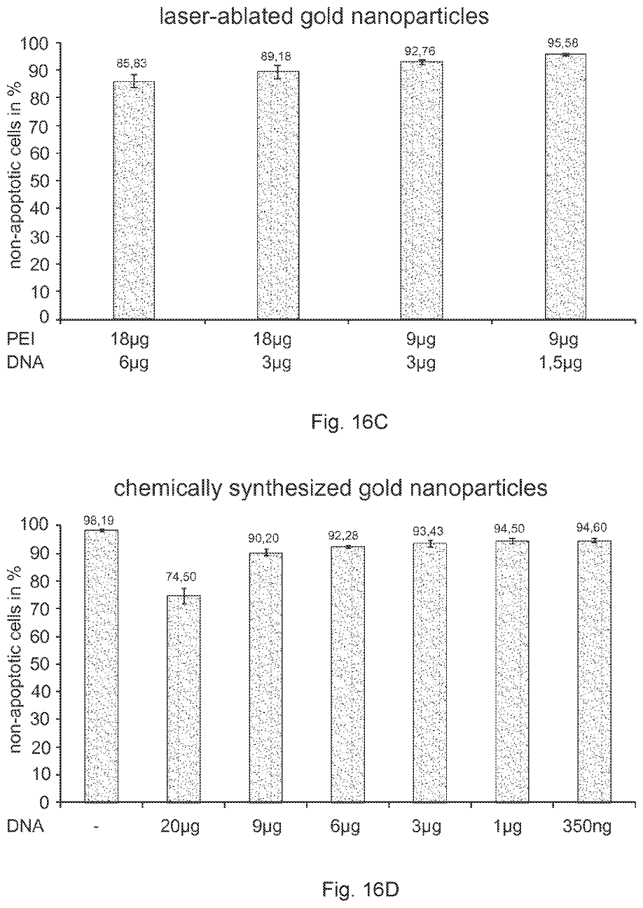

FIGS. 16A-16D shows the graphic representation of the results of studies in non-liver cell line HT1080, wherein conjugated gold nanoparticles obtained by laser ablation have been compared with comparative conjugated gold nanoparticles on the basis of chemically synthesized particles;

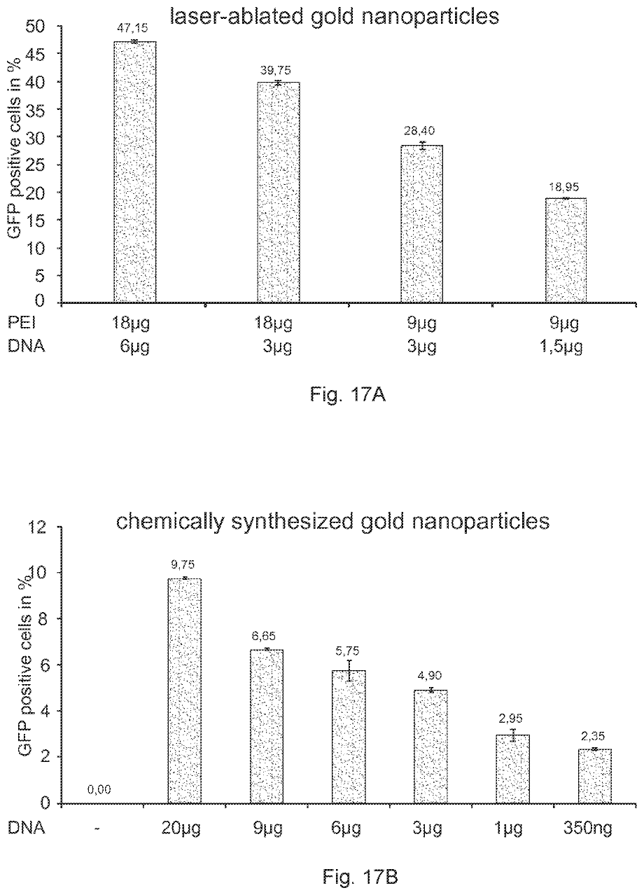

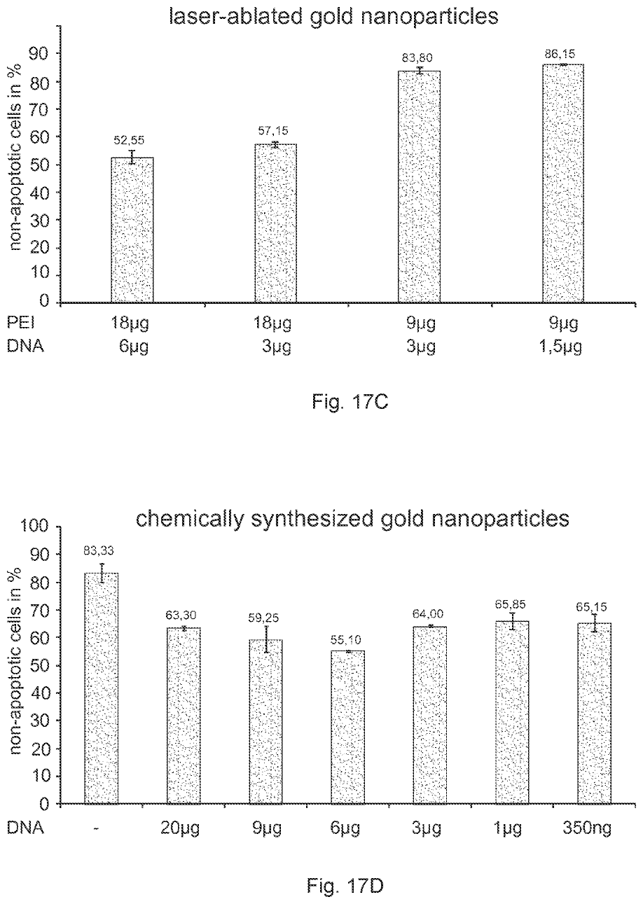

FIGS. 17A-17D shows the graphic representation of the results of studies in liver cancer cell line HLF, wherein conjugated gold nanoparticles obtained by laser ablation have been compared with comparative gold nanoparticles on the basis of chemically synthesized particles;

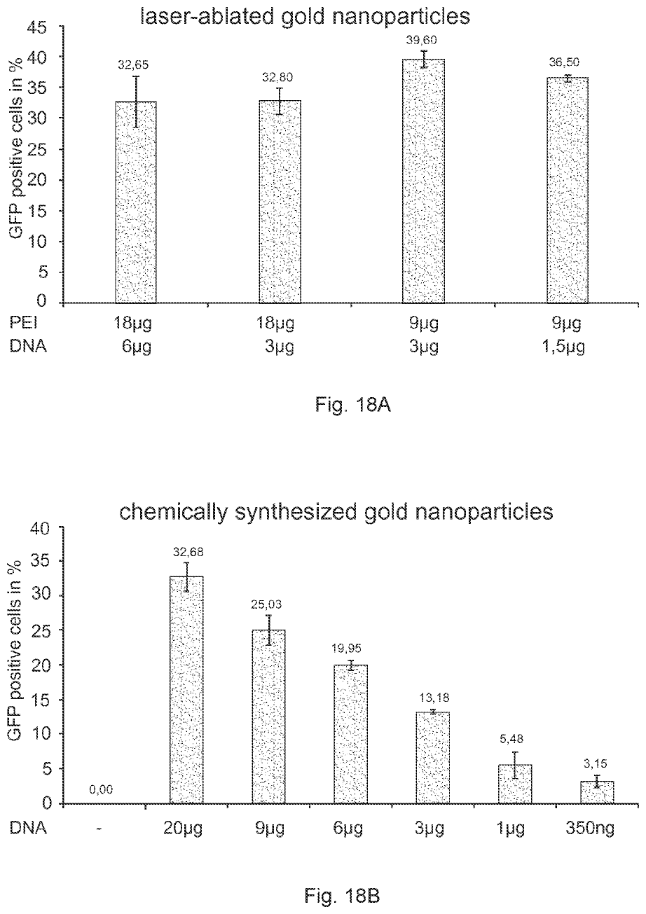

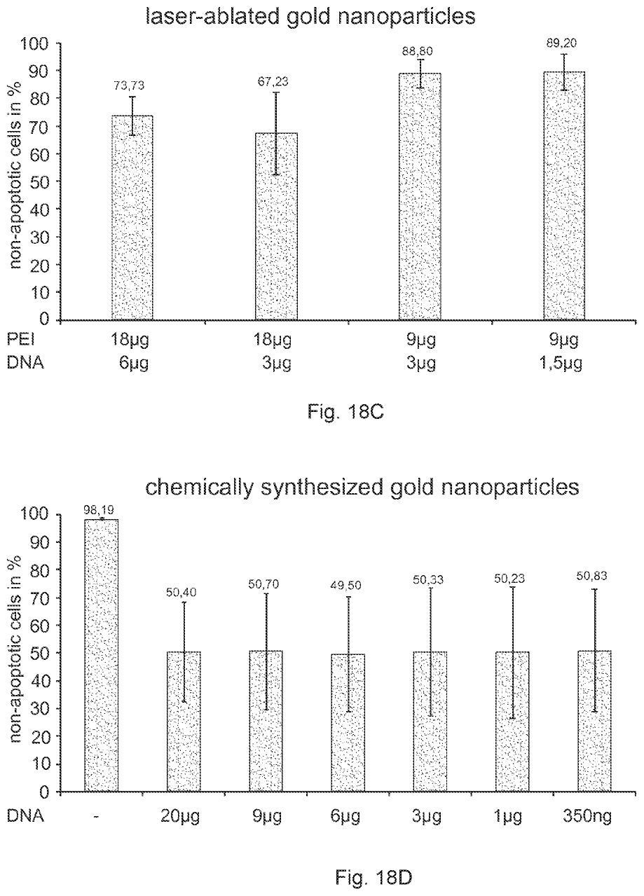

FIGS. 18A-18D shows the graphic representation of the results of studies in non-liver cell line HT1080, wherein conjugated gold nanoparticles obtained by laser ablation have been compared to comparative conjugated particles on the basis of chemically synthesized particles;

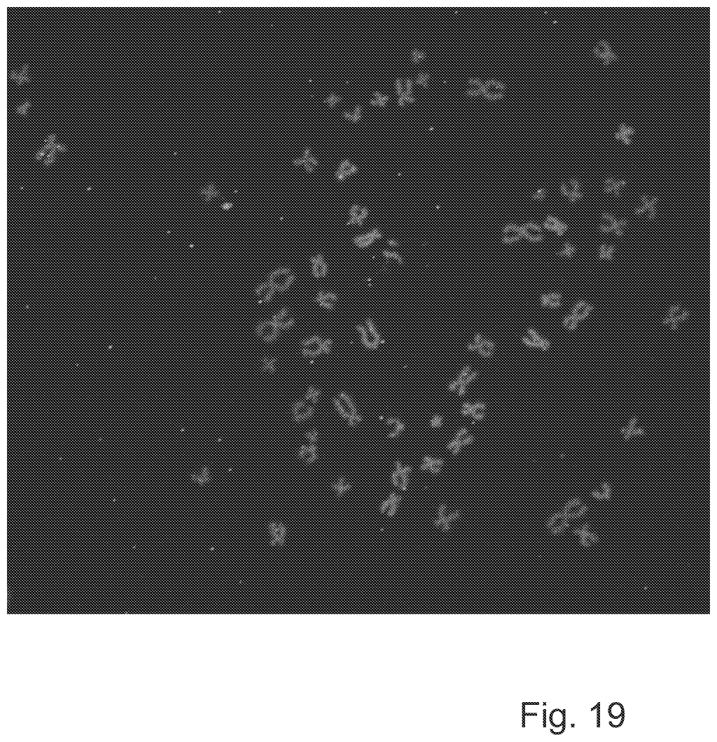

FIG. 19 shows an image obtained by FISH-analysis, wherein the episomal persistence of the transferred DNA mediated through the S/MAR element has been analyzed;

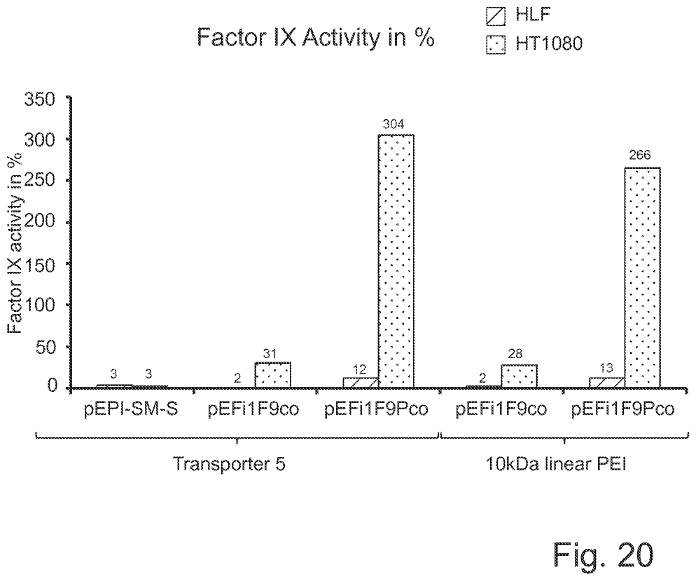

FIG. 20 shows the graphic representation of studies in fibrosarcoma cell line HT1080 and liver cancer cell line HLF, wherein the active factor level after transfection of the target cells with conjugated laser-ablated gold nanoparticles has been analyzed;

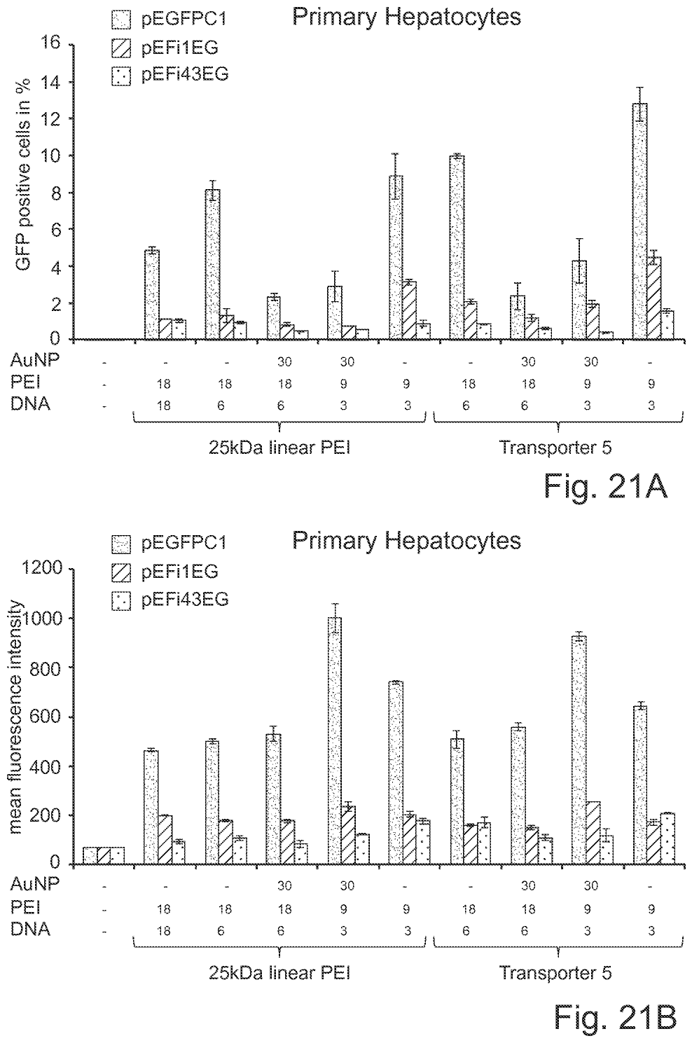

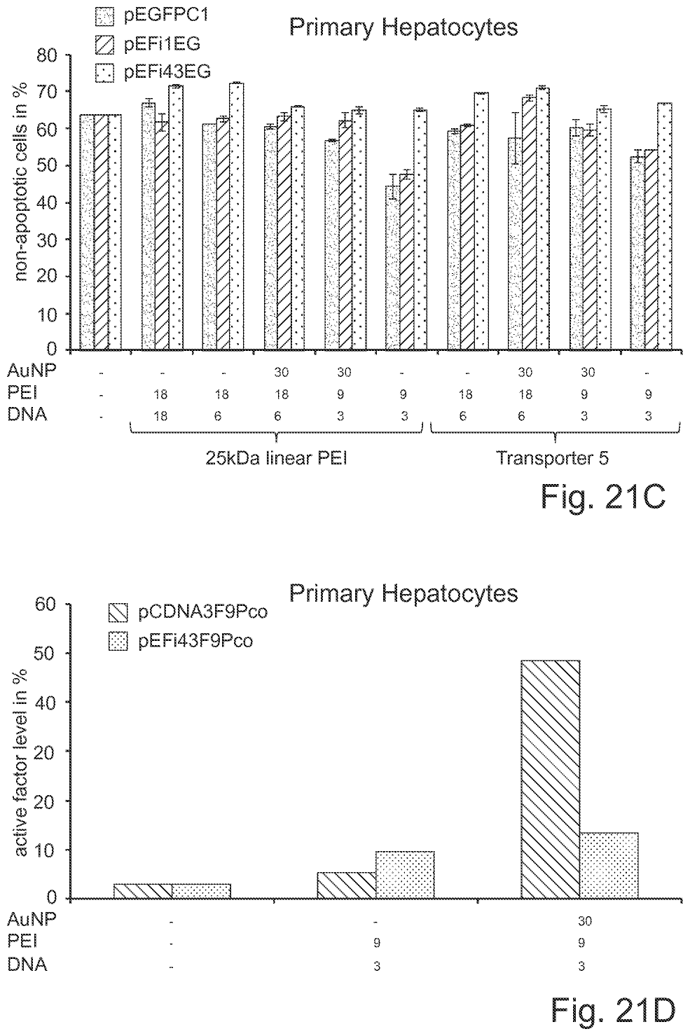

FIGS. 21A-21D shows the graphic representation of the results of studies in primary rat hepatocytes, wherein the gene transfer efficiencies and the active factor level of coagulation factor FIX have been analyzed;

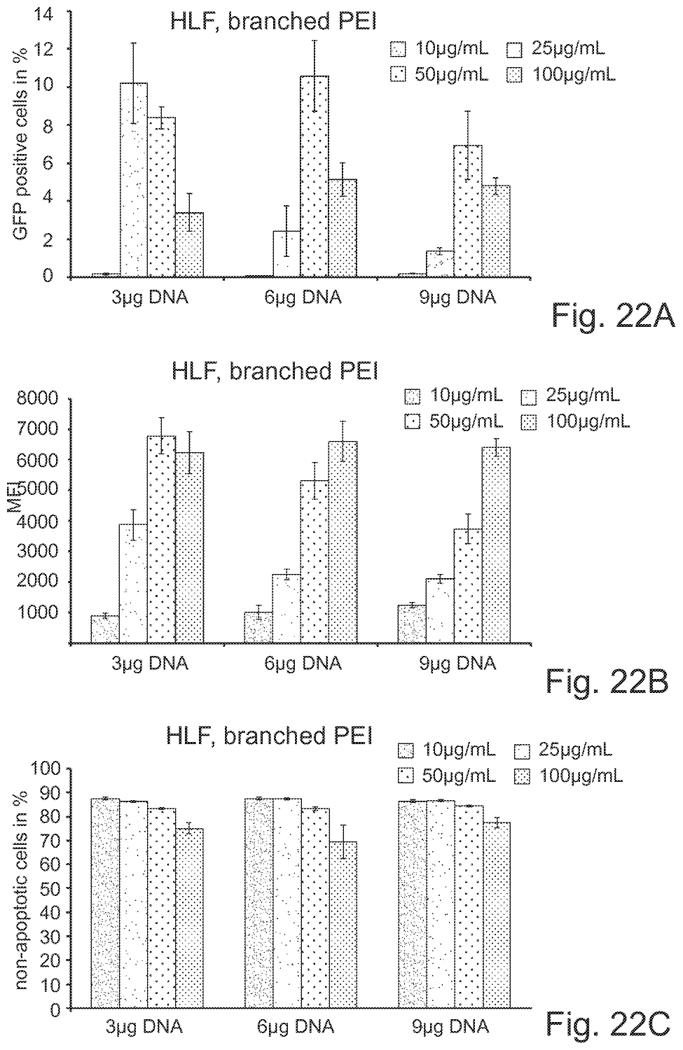

FIGS. 22A-22C shows the graphic representation of studies performed in HLF cells, wherein the transfection efficiency of conjugated gold nanoparticles generated on the basis of a preferred embodiment of the method according to the present invention has been analyzed, wherein the conjugation of the particles with polyethylenimine was performed simultaneously with laser ablation of the gold nanoparticles;

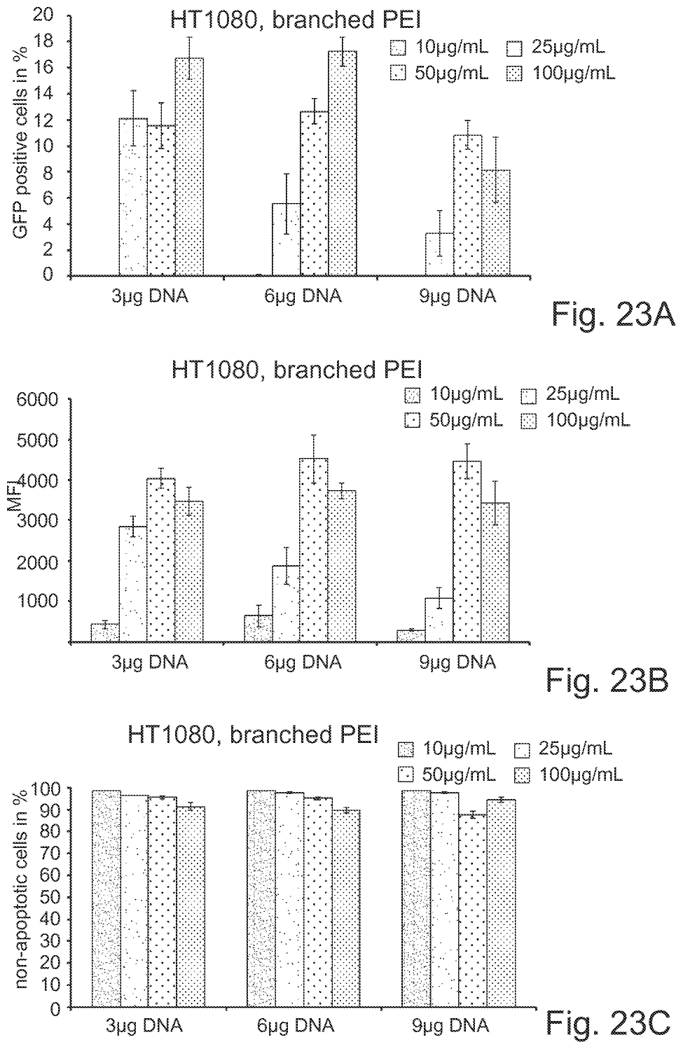

FIGS. 23A-23C shows the graphic representation of the results of studies performed in HT1080 fibrosarcoma cell line, wherein the transfection efficiency of conjugated gold nanoparticles generated by a preferred embodiment of the inventive method has been analyzed, wherein conjugation of the particles with polyethylenimine was performed simultaneously with laser ablation of the gold nanoparticles;

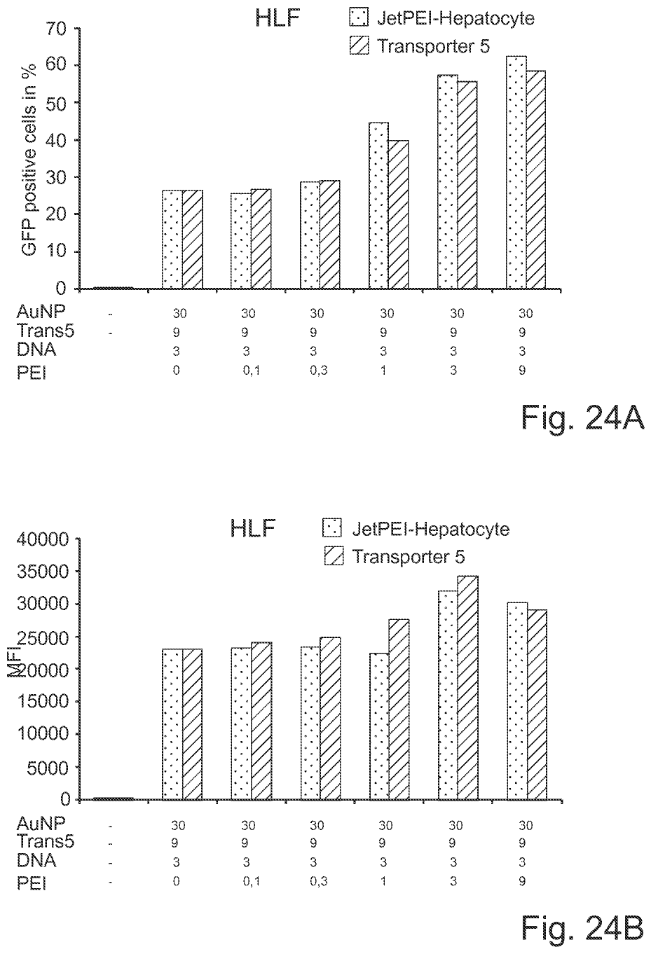

FIGS. 24A-24C shows the graphic representation of results performed in liver cancer cell line HLF, wherein the transfection efficiency of conjugated gold nanoparticles with a layer-by-layer assembly on the basis of an inner and an outer layer of polyethylenimine has been analyzed;

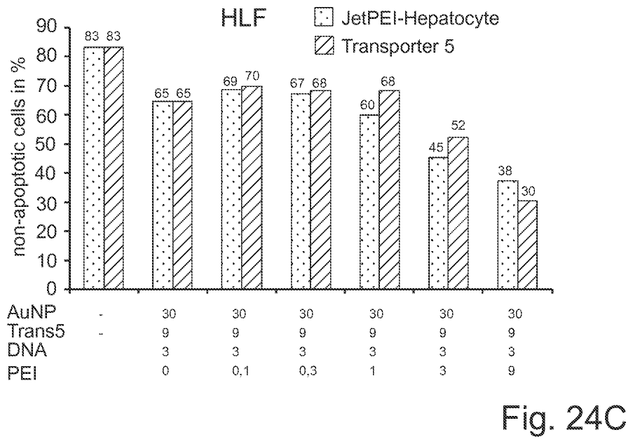

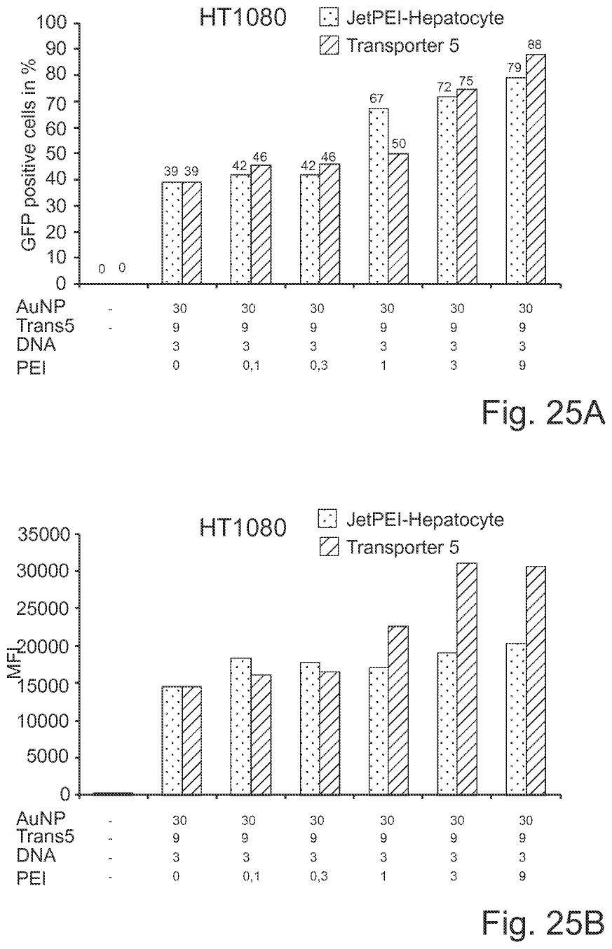

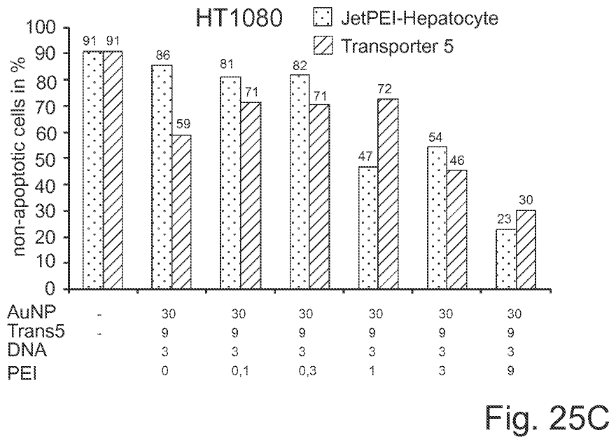

FIGS. 25A-25C shows the graphic representation of the results of studies in non-liver cell line HT1080, wherein the transfection efficiency of conjugated gold nanoparticles comprising a layer-by-layer assembly on the basis of an inner and an outer layer of polyethylenimine has been analyzed;

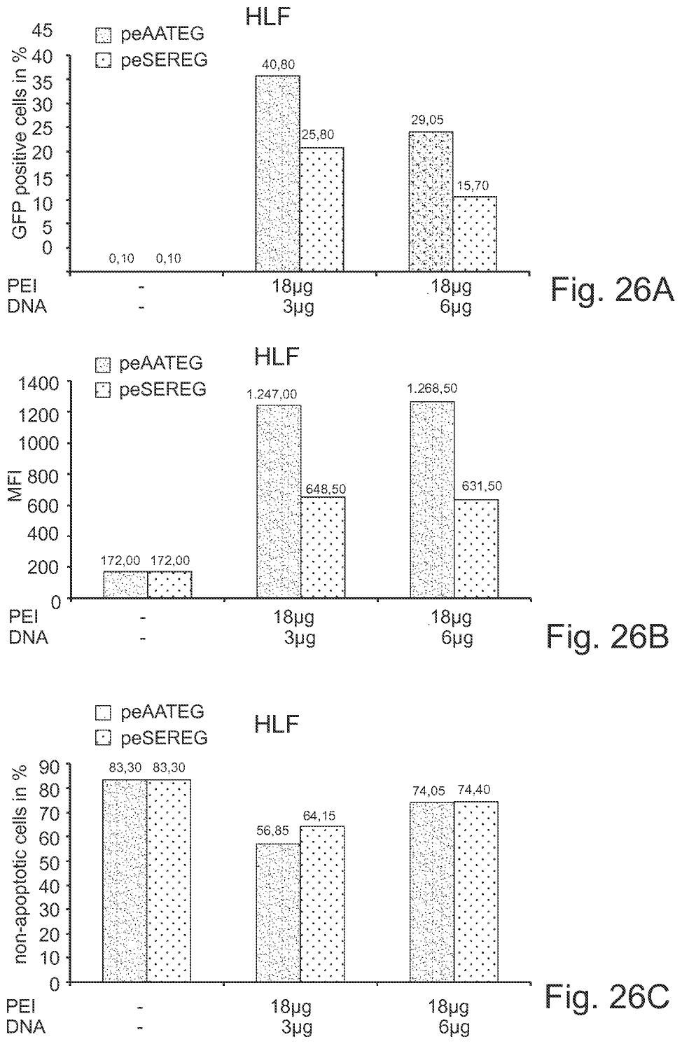

FIGS. 26A-26C shows the graphic representation of studies performed in HLF cells, wherein the transfection and expression efficiency of nucleic acid molecules containing either the hAAT-promoter or the SERPINA1-promoter has been analyzed;

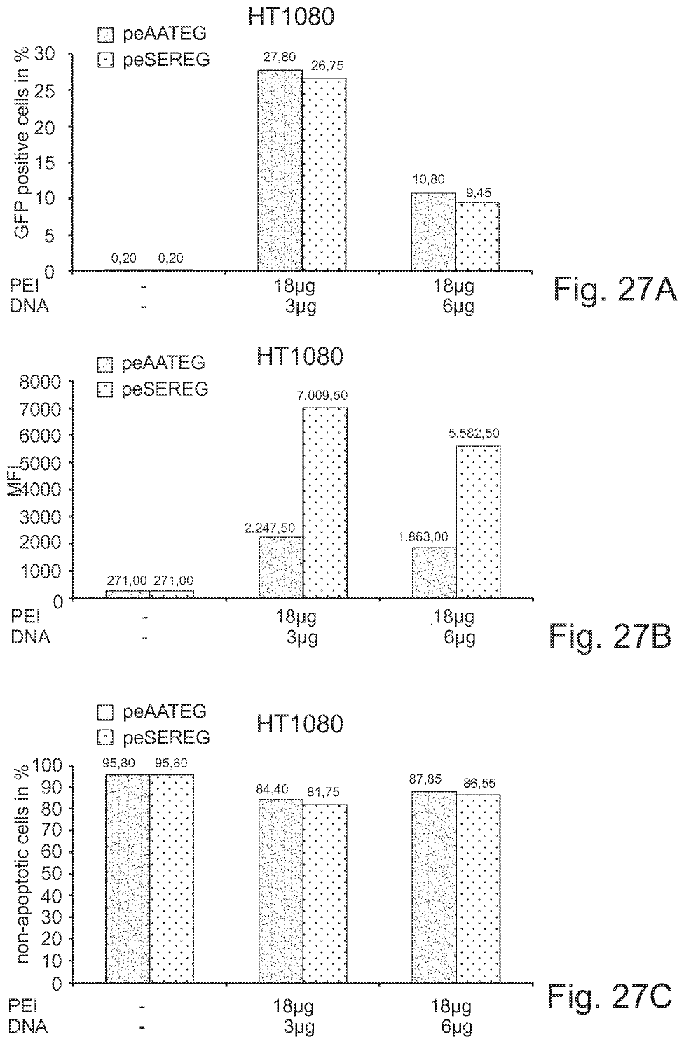

FIGS. 27A-27C shows the graphic representation of the result of studies performed in non-liver cell line HT1080, wherein the transfection and expression efficiency of nucleic acid molecules comprising the hAAt-promoter or the SERPINA1-promoter has been analyzed; and

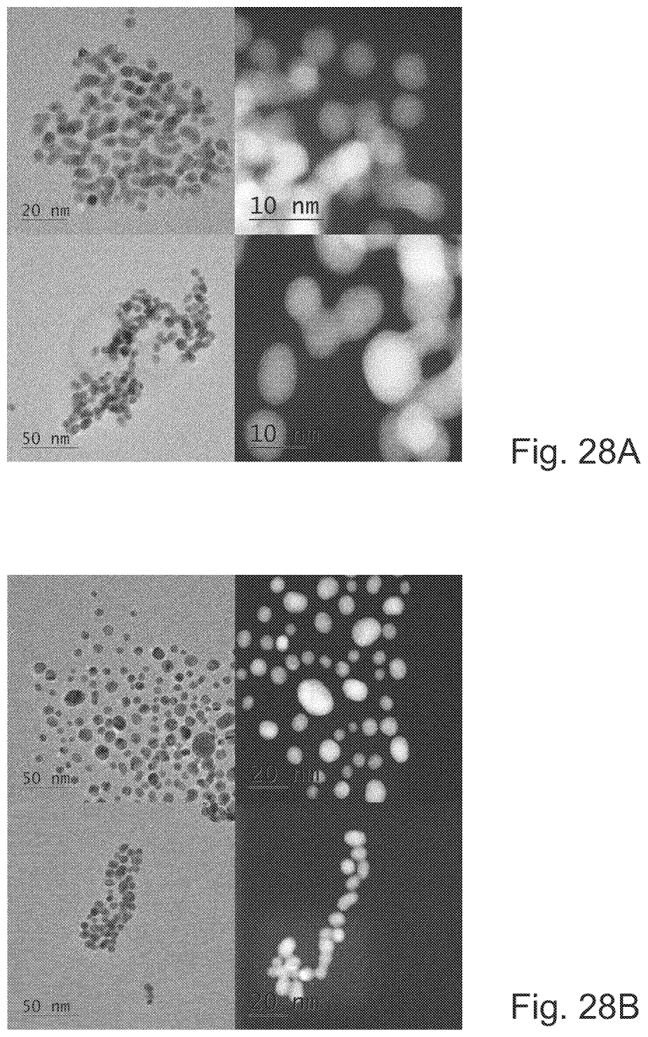

FIGS. 28A-28B shows images obtained by transmission electron microscopy (TEM) of gold nanoparticles obtained by PLAL and conjugated according to the present invention.

FIG. 1A shows a first preferred embodiment of conjugated gold nanoparticles according to the present invention, which is suitable for the transfer of nucleic acid molecules into eukaryotic cells, in particular human liver cells or fibrous tissue cells.

According to a preferred embodiment of the present invention, the conjugated gold nanoparticle 1 comprises a gold nanoparticle 2. The gold nanoparticle 2 comprises electrostatically bound polyethylenimine 3 and/or derivatives and/or salts thereof. In particular, the gold nanoparticle 2 is coated with polyethylenimine 3. Furthermore, on the basis of the polyethylenimine 3, nucleic acid molecules 4 are bound to the polyethylenimine/nanoparticle complex. On this basis, the polyethylenimine 4 fulfills several functions in the conjugated gold nanoparticles according to the present invention. On the one hand, the polyethylenimine 3 mediates the binding of the nucleic acid molecules 4 to the surface of the gold nanoparticles 2. On the other hand, polyethylenimine serves as the transfection reagent in order to improve the transfer of the nucleic acid molecules into the cells, in particular—without being bound to this theory—on the basis of the proton sponge effect, as delineated herein after in connection with FIG. 2.

FIG. 1B shows a preferred embodiment of conjugated gold nanoparticles 1 according to the present invention, wherein the conjugated gold nanoparticles comprise a layer-by-layer assembly on the basis of an inner and an outer polyethylenimine layer. Furthermore, FIG. 1B shows a schematic illustration of the process steps in order to prepare conjugated gold nanoparticles according to this preferred embodiment.

In this context, naked laser-ablated gold nanoparticles 2 are conjugated with a first polyethylenimine 3A, wherein this first polyethylenimine forms a first or inner layer on the surface of the gold nanoparticles. Subsequent to the first conjugation step, the polyethylenimine/gold nanoparticle complex is conjugated with nucleic acid molecules 4, which bind to the first polyethylenimine 3A. After adding the nucleic acid molecules 4, the conjugated gold nanoparticles 1, i.e. the polyethylenimine/gold nanoparticle/nucleic acid molecules complexes, are conjugated with a second polyethylenimine 3B and/or 3C. According to a preferred embodiment of the present invention, the second polyethylenimine 3C comprises a targeting unit, in particular on the basis of a conjugation with galactose. An outer layer on the basis of galactose-conjugated polyethylenimine allows a specific targeting of the conjugated gold nanoparticles to liver-cells, as delineated above. Likewise, the second polyethylenimine 3B can be identical to the polyethylenimine of the first and/or inner layer or any other of the above-mentioned polyethylenimines. Furthermore, the outer layer can also be based on a combination of a galactose-conjugated polyethylenimine and any other polyethylenimine used according to the present invention.

FIG. 2 shows on the basis of an illustration of a section of a target cell a schematic representation of the underlying concept of the transfer of nucleic acid molecules into the target cells, preferably liver cells, mediated by conjugated gold nanoparticles according to the present invention.

Starting point are conjugated gold nanoparticles according to the present invention, in particular as depicted in FIG. 1. In order to achieve a transfection of the target cells on the basis of a target cell 5, the conjugated gold nanoparticles 1 bind to the cell surface, in particular cell surface receptors, of the target cells, preferably.

The uptake of the conjugated gold nanoparticles into the cells occurs by endocytosis (B), resulting in the formation of an endosome 6 (C), which contains the conjugated gold nanoparticle 1 carrying the nucleic acid molecules 4 to be transferred. From the endosomes 6, the nucleic acid molecules 4 cannot directly enter the cytoplasm. On the basis of the polyethylenimine 3 bound to the gold nanoparticles 2, water molecules flow into the endosomes (D), causing the endosomes to burst (E). As a result, the nucleic acid molecules 4 to be transferred for transgenic expression of a coding sequence in the target cells are released into the cytoplasm (F).

The nuclear import (G) of the nucleic acid molecules 4 into the nucleus 9 then occurs passively during cell division after dissolution of the nuclear membrane or actively in non-dividing cells through nuclear pores 8 on the basis of transport molecules, in particular importins 7. In the nucleus 9, the nucleic acid molecules 4 bind to the core matrix and are replicated and expressed, resulting in the production of the liver-specific and/or liver-expressed protein.

With respect to a use in gene therapy, gold nanoparticles are mainly taken up by the liver after intravenous injection when used as carriers for nucleic acid sequences. Therefore, the conjugated gold nanoparticles according to the present invention comprise by nature a high specificity for the liver. According to the present invention, the binding of the conjugated nanoparticles to the surface of the liver cells is—without being bound to this theory—mediated by the transfection reagent on the basis of polyethylenimine. Since the conjugated gold nanoparticles according to the present invention as such already provide a high liver-specificity, a specific targeting is not necessarily needed in order to achieve a sufficient transfection of liver cells. Nevertheless, according to a particularly preferred embodiment of the present invention, galactose-conjugated polyethylenimine can be used for targeting.

FIGS. 3A to 3M contain schematic illustrations of expression vectors and/or plasmids constructed for in vivo experiments and/or transfection experiments in order to analyze the functionality of conjugated gold nanoparticles and vectors according to the present invention.

The vectors as illustrated in FIGS. 3A to 3N have been generated using standard cloning techniques (cf. also working examples).

The vectors pEPI1-SM-L as shown in FIG. 3A and pEPI1-SM-S as shown in FIG. 3B are based on the plasmid pEGFP-C1, which is commercially available from Clontec, Mountain View, Calif., US. Both vectors contain a promoter derived from cytomegalovirus (CMV) and a sequence coding for the enhanced Green Fluorescent Protein (eGFP) as a reporter gene. Furthermore, the vectors contain a neomycin/kanamycin resistance cassette in the plasmid backbone. The vector pEPI1-SM-1 according to FIG. 3A additionally contains a 1.995 base pair long scaffold/matrix attachment region (S/MAR) from the 5′ region of the human gene coding for Interferon-beta, in particular with a nucleic acid sequence according to SEQ ID NO. 19. The vector according to FIG. 3B contains in contrast to the vector according to FIG. 3B a shortened version of the S/MAR element derived from the human gene coding for Interferon-beta, in particular with a nucleic acid according to SEQ ID NO. 20.

The vector pEFi1-F9Pco as shown in FIG. 3C comprises a promoter derived from the promoter of the human elongation factor-1 alpha (EF1a), in particular a promoter according to SEQ ID NO. 2. Furthermore, the vector contains as the coding sequence a nucleotide sequence coding for coagulation factor FIX (Padua mutant), in particular a nucleotide sequence according to SEQ ID NO. 12. Furthermore, the vector contains for the purpose of selection a neomycin/kanamycin resistance cassette in the plasmid backbone.

The vector peSEREG as shown in FIG. 3D comprises as coding sequence a nucleotide sequence coding for the green fluorescent protein under transcriptional control of the SERPINA-1 promoter, preferably a promoter with a nucleic acid sequence according to SEQ ID NO. 5. Furthermore, upstream of the coding sequence and the promoter, the vector comprises a cis-regulatory element on the basis of the apolipoprotein E hepatic control region, in particular with a nucleotide sequence according to SEQ ID NO. 6.

The vector pcDNA3F9PwtInt1 as shown in FIG. 3E comprises a nucleotide sequence coding for coagulation factor FIX padua, in particular with a nucleotide sequence coding for a protein with an amino acid sequence according to SEQ ID NO. 14 under the control of the CMV promoter according to SEQ ID NO. 1.

The vector pcDNA3F9Pco as shown in FIG. 3F also comprises a nucleotide sequence coding for coagulation factor FIX padua, in particular a nucleotide sequence coding for a protein according to SEQ ID NO. 14, under the CMV promoter, preferably according to SEQ ID NO. 1.

The vector pcDNA3F9Pco_int1 according to FIG. 3G also comprises a nucleotide sequence coding for coagulation factor FIX padua, in particular coding for a protein with a amino acid sequence according to SEQ ID NO. 14, under the control of the CMV promoter according to SEQ ID NO. 1.

The vector pEFi43_F9Pco according to FIG. 3H comprises a coding sequence, which codes for coagulation factor FIX (padua mutant, i.e. a protein according to SEQ ID NO. 14) under the control of a promoter derived from human elongation factor-1 alpha (EF1a), in particular according to SEQ ID NO. 2.

The vector pEFi43F9Pwtint1 as shown in FIG. 3I comprises a coding sequence coding for coagulation factor FIX padua, i.e. a protein having an amino acid sequence according to SEQ ID NO. 14, wherein the coding sequence is under the transcriptional control of a promoter derived from human elongation factor-1 alpha, in particular a promoter according to SEQ ID NO. 2.

The vector pEFi43F9PcoInt1 as shown in FIG. 3J comprises a coding sequence coding for coagulation factor FIX padua, i.e. a protein having an amino acid sequence according to SEQ ID NO. 14, wherein the nucleic acid sequence of the coding sequence is codon optimized for human codon usage. Furthermore, the coding sequence is under control of the promoter derived from human elongation factor-1 alpha, in particular according to SEQ ID NO. 2.

The vector pEFi43F9PcoI2EG as shown in FIG. 3K comprises as the coding sequence a nucleotide sequence coding for coagulation factor FIX padua, in particular a protein having an amino acid sequence according to SEQ ID NO. 14. Furthermore, the vector contains an IRES2 sequence (internal ribosome entry site 2) according to SEQ ID NO. 21 together with the sequence coding for the green fluorescent protein (GFP). The coding sequence is under control of the promoter derived from human elongation factor-1 alpha, in particular according to SEQ ID NO. 2.

The vector pEFi43F9PcoT2AEG as shown in FIG. 3L comprises a coding sequence coding for a fusion protein of coagulation factor FIX padua (amino acid sequence according to SEQ ID NO. 14) and GFP under the control of the promoter derived from human elongation factor-1 alpha, in particular according to SEQ ID NO. 2. Furthermore, the vector comprises a nucleic acid sequence coding for the 2A self-cleaving peptide of Thosea asigna virus (T2A) according to SEQ ID NO. 22.

The vector peAATEG as shown in FIG. 3M comprises a nucleotide sequence coding for GFP under the control of a promoter derived from human alpha 1 antitrypsin (hAAT), in particular with a nucleic acid sequence according to SEQ ID NO. 4. Furthermore, upstream of the promoter and the coding sequence, this vector comprises a cis-regulatory element on the basis of a apolipoprotein E hepatic locus control region, in particular according to SEQ ID NO. 6.

FIG. 4 shows a graphic representation of the result of studies performed in liver cancer cell line HLF, wherein the effect of the presence of S/MAR elements on the long-term expression level of the reporter gene coding for eGFP has been analyzed. In this context, the expression of eGFP on the basis of the vector pEPI-SM-L (cf. FIG. 3A) has been compared with the expression of eGFP on the basis of the vector pEPI1-SM-S (cf. FIG. 3B). Furthermore, the vector pEGFP-C1 with GFP under the transcriptional control of the CMV promoter and without any S/MAR element has been used as control. For this purpose, 300,000 cells in a 6-well format have been transfected with 6 μg DNA by using 18 μg branched PEI with a molecular weight of 25 kDa. Cells were splitted twice per week at a ratio of 1:15 and GFP expression levels were analyzed once per week by flow cytometry. Since liver cancer cell lines are fast dividing cells, in order to ensure the stability of the vector DNA in the cells, geneticin (G418) has been used for selection.

FIG. 4A shows the results of the GFP expression in a test series, where a short-term selection with G418 for about two weeks was applied. FIG. 4B contains the results of the test series where a long-term selection over the whole observation time of nine weeks with G418 has been applied. In this context, it can be seen that both variants of the S/MAR element, i.e. the long as well as the shortened variant, led to a long-term expression of eGFP in the transfected cells, which is superior compared to the expression of eGFP on the basis of a plasmid pEGFP-C1 containing eGFP under control of the same promoter but without S/MAR element. Both variants of the S/MAR element ensure an episomal persistence of the transferred nucleic acid molecules in the target cells, as can deduced from the expression of eGFP over the whole observation time. Furthermore, the shortened variant leads to a higher percentage of GFP positive cells, indicating an improved episomal persistence of the transferred nucleic acid molecules in the cells.

FIG. 5 shows the graphic representation of the results of studies in liver cancer cell line HLF using conjugated gold nanoparticles for the transfection of the target cells. In this context, the optimal variant of the S/MAR element was further investigated in connection with conjugated gold nanoparticles obtained by laser ablation. For this purpose, HLF cells were transfected with conjugated laser-ablated gold nanoparticles having an average particle diameter of 5 nm, determined on the basis of analytical disk centrifugation. The conjugated gold nanoparticles comprised as nucleic acid molecules either the vector pEGFP-C1 (control vector, comprising eGFP under the control of the CMV-promoter), pEPI-SM-S (cf. FIG. 3B) or pEPI-SM-L (cf. FIG. 3A). For the purpose of transfection, the HLF cells where transfected with conjugated gold nanoparticles comprising branched PEI with a molecular mass of 25 kDa and one of the aforementioned vectors. For transfection, 300,000 cells were seeded in a 6-well format and transfected with 6 μg DNA, 18 μg branched PEI with a molecular mass of 25 kDa and 30 μg of gold nanoparticles per well. In order to ensure a stable conjugation of the PEI to the nanoparticles, PEI and gold nanoparticles were pre-incubated the day before transfection and dialyzed against purified water with a 50 kDa molecular weight cut-off. Cells were splitted twice per week at a ratio of 1:15 and GFP expression levels were assessed once per week by flow cytometry.

FIG. 5A contains the results of a test series under short term selection with G418, wherein the selection has been performed during the first two weeks of cultivation. After two weeks, the cultivation in the presence of G418 was stopped for the rest of the observation time. As can be seen from FIG. 5A, both variants of the S/MAR element led to a long-term expression of eGFP in the transfected cells after short term selection. A higher percentage of eGFP positive cells is surprisingly achieved with the shortened variant of the S/MAR element. FIG. 5B contains the results of the test series where a long-term selection with G418 has been performed during the whole observation time of ten weeks. As can be seen from FIG. 5B, both variants of the S/MAR element led to a long-term expression of eGFP in the transfected cells under long-term selection with G418.

FIG. 6 shows the graphic representation of the results of studies in liver cell lines HLF and HepG2, wherein the influence of the particle size (average particle diameter) of the gold nanoparticles, i.e. the diameter of the laser-ablated gold nanoparticles before conjugation, has been analyzed. For this purpose, two different sizes of the laser-ablated gold nanoparticles, namely 5 nm and 50 nm, have been used in the conjugated gold nanoparticles for transfection. In order to measure the transfection efficiency, the gold nanoparticles were conjugated with the vectors pEPI-SM-S (cf. FIG. 3B) and pEPI-F8-SM-S (not shown, CMV promoter, coding sequence for factor FVIII-GFP fusion, short S/MAR element). For this purpose, 200,000 per well in a 6-well format were transfected by adding conjugated gold nanoparticles on the basis of 20 μg DNA, 30 μg gold nanoparticles and 18 μg of 25 kDa branched PEI per well. For an efficient conjugation, PEI and gold nanoparticles were pre-incubated the day before transfection and dialyzed against purified water with a 50 kDa molecular weight cut-off. As negative control, cells have been transfected without gold nanoparticles, wherein the same amount nucleic acid molecules and polyethylenimine has been used.

FIG. 6A shows the result of the analysis of the eGFP expression in HLF cells. In this context, it can be seen that particularly good results are achieved with gold nanoparticles with an average diameter of 5 nm in the unconjugated state. FIG. 6B shows the result of the analysis of the eGFP expression in HepG2 cells. Transfection efficiency in HepG2 cells was also higher with particles having a diameter of 5 nm. Overall, on the basis of the smaller particles a higher transfection efficiency is achieved.

FIG. 7 shows the graphic representation of the results of studies in liver cancer cell line HLF, wherein the impact of the weight related ratio of nucleic acid molecules to PEI on the transfection efficiency has been analyzed. For this purpose, the vector pEPI-SM-S according to FIG. 3B has been used. Furthermore, two different types of polyethylenimine, namely linear polyethylenimine with a molecular weight of 25 kDa, on the one hand, and branched polyethylenimine with a molecular weight of also 25 kDa, on the other hand, were used and compared with respect to the transfection efficiency. Nucleic acid molecules were used in amounts of 0.7 μg, 1.5 μg, 3 μg and 10 μg per well/approach. The amount of polyethylenimine was 9 μg per well. For the purpose of analysis, 200,000 cells per well were transfected by mixing the afore-mentioned DNA amounts with 9 μg of the branched or the linear polyethylenimine. Cells were analyzed for GFP expression three days after transfection by flow cytometry.

FIG. 7A shows the GFP expression, measured on the basis of the percentage of GFP positive cells after transfection of the pEPI-SM-S vector. In this context, it can be seen that the highest GFP expression level is observed with a PEI:DNA ratio of 3:1. Furthermore, an overall higher GFP expression is achieved on the basis of linear PEI. The weakest GFP expression is achieved with a PEI:DNA ratio of 9:10 (1:1,11). FIG. 7B contains the GFP expression measured on the basis of the mean fluorescence intensity (MFI) of the GFP positive cells. In this context, the branched polyethylenimine led to a stable expression independent of the DNA amount, while the linear polyethylenimine required higher DNA amounts for a stable expression of the coding sequence. FIG. 7C contains the result of the determination of the cell viability on the basis of the percentage of non-apoptotic cells. As can be seen from FIG. 7C, the cells viability in all test series with all tested weight related ratios and both variants of polyethylenimine was satisfying. The toxicity of branched PEI was slightly higher when compared to linear PEI, but still satisfying.

FIG. 8 shows the graphic representation of the results of studies performed in the fibrosarcoma cell line HT1080, wherein the influence of the weight related ratio of DNA to PEI has been analyzed. The approach was identical to the approach described in connection with FIG. 7, with the exception of the cell type.

FIG. 8A contains the results with respect to the GFP expression on the basis of the percentage of GFP positive cells. As can be seen from FIG. 8A, both PEI variants show the highest level of GFP positive cells at divergent ratios of PEI to DNA. For branched PEI, a ratio of polyethylenimine to DNA of 3:1 led to the highest transgene expression, while a ratio of about 12:1 seems to be more favorable for linear PEI. FIG. 8B shows the graphic representation of the GFP expression on the basis of the mean fluorescence intensity (MFI) of the GFP positive cells. In this context, it can be seen that higher DNA amounts led to a higher GFP expression, independent from the PEI variant. FIG. 8C contains the results of the determination of the cell viability on the basis of the percentage of non-apoptotic cells. As can be seen from FIG. 9C, the cell viability in all tested series with all tested weight related ratios and both variants of polyethylenimine was satisfying.

FIG. 9 shows the graphic representation of the results of studies performed in liver cancer cell line HLF, wherein the influence of the weight related ratio of nucleic acid molecules to transfection agent in conjugated laser-ablated gold nanoparticles on the transfection efficiency has been analyzed. In this context, the eGFP transgene expression has been determined. For this purpose, cells of the liver cancer cell line HLF have been transfected with the vector pEPI-SM-S (cf. FIG. 3B), wherein transfection was performed on the basis of conjugated gold nanoparticles with an average particle diameter of 5 nm, different amounts of nucleic acid molecules and different variants of polyethylenimine as transfection reagent. In this context, linear polyethylenimine with a molecular mass of 25 kDa and branched polyethylenimine with a molecular weight of 25 kDa have been used. The transfection agents were used in an amount of 9 μg per well. The amount of DNA in the conjugated gold nanoparticles was 0.7 μg, 1.5 μg, 3 μg or 10 μg. Gold nanoparticles have been used in an amount of 30 μg per well. For the purpose of transfection, the liver cells have been mixed with the conjugated gold nanoparticles. In particular, conjugated gold nanoparticles were prepared by mixing the DNA amounts with 30 μg gold nanoparticles having an average particle diameter of 5 nm and 9 μg of the respective polyethylenimine variant. With respect to the conjugation, polyethylenimine and gold nanoparticles were pre-incubated the day before transfection and dialyzed against water with a 50 kDa molecular weight cut-off. The conjugated gold nanoparticles were mixed with the cells, wherein each well contained 200,000 cells. GFP expression was analyzed three days after transfection by flow cytometry.

FIG. 9A contains the results concerning the percentage of GFP positive cells. In this context, it can be seen that the highest GFP expression levels were observed with a weight related ratio of polyethylenimine to nucleic acid molecules of 3:1. Furthermore, a higher transfection efficiency was achieved with linear polyethylenimine. In addition, the mean fluorescence intensities (MFIs) have been determined. The respective results are depicted in FIG. 9B. Furthermore, the cell viability on the basis of the determination of the percentage of non-apoptotic cells has been analyzed three days after transfection. As can be seen from FIG. 9C, the transfection with all variants of polyethylenimine in different weight related ratios to the nucleic acid molecules is linked with sufficient cell viability.

FIG. 10 shows the graphic representation of the result of studies performed in fibrosarcoma cell line HT1080, wherein the influence of the weight related ratio of polyethylenimine to nucleic acid molecules in the conjugated gold nanoparticles on the transfection efficiency has been analyzed. The approach was identical to the approach described in connection with FIG. 9, with the exception of the cell type. FIG. 10A shows the percentage of GFP positive cells three days after transfection. It can be seen from FIG. 10A that both variants of polyethylenimine achieved the highest percentage of GFP positive cells at different ratios of polyethylenimine to nucleic acid molecules. With respect to the branched polyethylenimine, a ratio of polyethylenimine to nucleic acid molecules of 3:1 led to the highest expression levels of GFP, whereas for the linear polyethylenimine the ratio of about 12:1 seemed to be more favorable. Nevertheless, also with a ratio of 3:1 or 6:1 sufficient results have been achieved. FIG. 10B shows the results of the mean fluorescence intensity (MFI) of the GFP positive cells. In this context it can be seen that for both polyethylenimine variants, higher mean fluorescence intensities correlated with higher amounts of nucleic acid molecules. FIG. 10C shows the results of an analysis of the cell viability on the basis of the determination of the percentage of non-apoptotic cells three days after transfection. It can be seen from FIG. 10C that all variants of polyethylenimine as well as all tested amounts of DNA used for transfection led to sufficient viability of the transfected cells.

FIG. 11 shows the graphic representation of the result of studies performed in liver cancer cell line HLF, wherein the influence of different linear PEI variants on the transfection efficiency has been analyzed. For this purpose, the following four different transfection reagents have been used: 25 kDa linear polyethylenimine, 10 kDA linear polyethylenimine, JetPEI® (linear PEI, commercially available from Polyplus Inc., Illkirch, FR) and Transporter5™ (linear PEI, commercially available from Polysciences Europe GmbH, Hirschberg an der Bergstraße, DE). Furthermore, two different amounts of polyethylenimine (9 μg and 18 μg per well) and two different weight related ratios of polyethylenimine to nucleic acid molecules (3:1 and 6:1) have been tested. Transfection has been performed with the vector pEPI-SM-S according to FIG. 3B. For the purpose of transfection, the indicated amounts of polyethylenimine and nucleic acid molecules were mixed and incubated with 200,000 cells per well of a 6-well plate. GFP expression has been analyzed three days after transfection by flow cytometry.

FIG. 11A contains the results concerning the percentage of GFP positive cells in the different approaches. In this context, it can be seen that all variants of polyethylenimine in combination with all amounts of nucleic acid molecules led to sufficient transfection of HLF cells with the vector. The best results are achieved with respect to the transfection efficiency with Transporter5™ as transfection agent. The results are further confirmed by the results of the determination of the mean fluorescence intensity (MFI) of eGFP in the GFP positive cells, which are depicted in FIG. 11B. Furthermore, the cell viability on the basis of the determination of the percentage of non-apoptotic cells has been analyzed three days after transfection. As can be seen from FIG. 11C, the use of Transporter5™ is linked with the lowest toxicity. Furthermore, the double amount of polyethylenimine and nucleic acid molecules is associated with higher toxicity.

FIG. 12 shows the graphic representation of the result of studies performed in fibrosarcoma cells HT1080, wherein the influence of different linear PEI variants on transfection efficiency and toxicity has been analyzed. The approach was identical to the approach described before in connection with FIG. 11, with the exception of the cell type.

FIG. 12A shows the results concerning the percentage of GFP positive cells in the different approaches. In this context, it can be seen that the highest population of GFP expressing cells was achieved with 25 kDA linear PEI at a 6:1 ratio of polyethylenimine to nucleic acid molecules. This ratio was also favorable for the other variants of polyethylenimine. The double amounts of polyethylenimine and nucleic acid molecules do not lead to larger populations of cells expressing GFP. This applies to all tested polyethylenimine variants. FIG. 12B contains the results of the mean fluorescence intensity of the GFP expressing cells. As can be seen from FIG. 12B, the highest mean fluorescence intensity level was achieved with JetPEI® as transfection agent at a 3:1 weight related ratio of polyethylenimine to nucleic acid molecules, even though the MFI values do not very much with other concentrations of ratios. This also applies to the other variants of polyethylenimine. Furthermore, the cell viability on the basis of the determination of the percentage of non-apoptotic cells have been analyzed three days after transfection. As can be seen from FIG. 12C, the transfection with all variants of polyethylenimine is linked with a sufficient cell viability.

FIG. 13 shows the graphic representation of the results of studies in liver cancer cell line HLF, wherein the cells have been transfected with conjugated laser-ablated gold nanoparticles. In this context, gold nanoparticles with an average particle diameter of 5 nm have been used in combination with four different transfection reagents, i.e. 25 kDa linear polyethylenimine, 10 kDa linear polyethylenimine, linear JetPEI® (commercially available from PolyPlus Inc., Illkirch, FR) and linear Transporter5™ (commercially available from Polysciences Europe GmbH, Hirschberg an der Bergstraße, DE). The conjugated gold nanoparticles comprise two different quantities of polyethylenimine (9 μg and 18 μg per well) and two different weight related ratios of polyethylenimine to DNA (3:1 and 6:1). For the purpose of analysis, the test vector pEPI-SM-S (cf. FIG. 3B) has been used as nucleic acid molecules for the transfection experiments. For transfection, 30 μg gold nanoparticles have been conjugated with the aforementioned variants of polyethylenimine in an amount of 18 μg or 9 μg, respectively, and nucleic acid molecules in amounts of 1.5 μg, 3 μg or 6 μg. In this context, the polyethylenimine variants and the gold nanoparticles were pre-incubated the day before transfection in order to allow the conjugation of the gold nanoparticles with the transfection agent. After incubation, the conjugated gold nanoparticles were dialyzed against water with a 50 kDa molecular weight cut-off. Afterwards, further conjugation of the particles with nucleic acid molecules has been performed by admixing the nucleic acid molecules to the pre-conjugated particles. The conjugated gold nanoparticles were incubated with 200,000 cells per well of a 6-well plate and eGFP expression was analyzed three days after transfection by flow cytometry.

FIG. 13A contains the results concerning the percentage of GFP positive cells in the different approaches. In this context, the highest amount of GFP expressing cells was achieved with 10 kDA polyethylenimine at a 6:1 ratio of polyethylenimine to nucleic acid molecules. In general, higher quantities of polyethylenimine led to larger GFP positive cell populations. Furthermore, FIG. 13B contains the results with respect to the mean fluorescence intensity levels (MFI) of the GFP positive cells. The highest MFI values were obtained with Transporter5™ and 10 kDa linear polyethylenimine as transfection reagents and with the highest concentration of DNA of 6 μg/well. Furthermore, the cell viability on the basis of the determination of the percentage of non-apoptotic cells has been analyzed three days after transfection. As can be seen from FIG. 13C, all tested approaches led to a sufficient cell viability, even though higher amounts of polyethylenimine and nucleic acid molecules are associated with slightly more toxicity.

FIG. 14 shows the graphic representation of the results of studies performed in fibrosarcoma cell line HT1080, wherein the approach was identical to the approach described before in connection with FIG. 13 with the exception of the cell type. FIG. 14A contains the results concerning the percentage of GFP positive cells in the different approaches. The highest amount of GFP expressing cells was achieved with JetPEI® with a weight related ratio of polyethylenimine to nucleic acid molecules of 3:1. Except for linear polyethylenimine with a molecular mass of 10 kDa, higher amounts of polyethylenimine and nucleic acid molecules resulted in higher GFP expression levels for all tested variants of polyethylenimine. The results are further confirmed by the results of the determination of the mean fluorescence intensity (MFI) of eGFP in the GFP positive cells, which are depicted in FIG. 14B. Furthermore, the cell viability on the basis of the determination of the percentage of non-apoptotic cells has been analyzed. As can be seen from FIG. 14C, except for the linear 10 kDa polyethylenimine, not much toxicity or apoptosis was observed when transfecting HT1080 cells with conjugated laser ablated gold nanoparticles.

FIG. 15 shows the graphic representation of the results of studies in liver cancer cell line HLF, wherein the gene transfer efficiencies of conjugated laser-ablated gold nanoparticles and of conjugated chemically synthesized gold nanoparticles have been compared. For this purpose, conjugated gold nanoparticles according to the present invention on the basis of laser-ablated gold nanoparticles with a size of 5 nm and 25 kDa linear PEI and the vector pEPI1-SM-S (cf. FIG. 3B) have been compared with conjugated gold nanoparticles on the basis of chemically synthesized gold nanoparticles with a size of 5 nm, covalently bound 25 kDa linear PEI and the vector pEPI1-SM-S as nucleic acid molecules. Furthermore, as a negative control, transfection was also performed with 25 kDa polyethylenimine as transfection reagent for the nucleic acid molecules. The negative control was based on untransfected cells.

According to this approach, 25 kDa linear PEI was used in two different amounts, i.e. 18 μg and 9 μg, and two different weight related ratios of polyethylenimine to nucleic acid molecules, i.e. 3:1 and 6:1 per well. For conjugation, gold nanoparticles with an average particle diameter of 5 nm (generated by pulsed laser ablation in liquid) and linear 25 kDa polyethylenimine (commercially available from Sigma-Aldrich/Merck KGaA, Darmstadt, DE) were pre-incubated the day before transfection and dialyzed against water with a 50 kDa molecular weight cut-off. Chemically synthesized gold nanoparticles with an average particle diameter of 5 nm and covalently bound 25 kDa linear PEI have been obtained from Nanopartz Inc., Loveland, Colo., US. For further conjugation of the gold nanoparticles with the vector DNA, the nucleic acid molecules have been added to the laser-ablated particles in an amount of 1.5 μg, 3 μg and 6 μg per well. Chemically synthesized gold nanoparticles were further conjugated with 350 μg, 1 μg, 3 μg, 6 μg or 20 μg nucleic acid molecules. The conjugated gold nanoparticles were incubated with 200,000 cells per well of a 6-well plate and GFP expression was analyzed three days after transfection by flow cytometry.

FIG. 15A contains the result concerning the percentage of GFP positive cells transfected with laser-ablated gold nanoparticles and non-inventive, chemically synthesized gold nanoparticles. It can be seen from FIG. 15A that conjugated gold nanoparticles on the basis of laser-ablated particles led to a significantly higher transfection efficiency when compared to conjugated gold nanoparticles on the basis of chemically synthesized particles. With the conjugated gold nanoparticles obtained by laser ablation, 16.17% to 35.85% GFP positive cells have been obtained by transfection. In contrast to this, chemically synthesized nanoparticles led only to 0.15% to 0.38% GFP positive cells. Furthermore, the cell viability on the basis of the determination of the percentage of non-apoptotic cells has been analyzed three days after transfection. As can be seen from FIG. 15B, both approaches led to a sufficient cell viability, even though toxicity of the chemically synthesized gold nanoparticles in HLF cells was slightly lower. Nevertheless, the cell viability achieved with the conjugated gold nanoparticles according to the present invention is still sufficient.

FIG. 16 shows the graphic representation of the results of studies performed in non-liver cell line HT1080. The approach was identical to the approach described in connection with FIG. 15, except for the cell line. FIG. 16A contains the results concerning the percentage of GFP positive cells in the different approaches. In this context, it can be seen that laser-ablated gold nanoparticles in the conjugated gold nanoparticles are largely superior with respect to the transfection efficiency when compared to conjugated gold nanoparticles on the basis of chemically synthesized gold nanoparticles. In this context, on the basis of the conjugated gold nanoparticles according to the present invention, transfection led to 48.13% to 70.91% GFP positive cells. In contrast to this, the chemically synthesized gold nanoparticles led to 0.65% to 3.25% GFP positive cells. Furthermore, the cell viability on the basis of the determination of the percentage of non-apoptotic cells has been analyzed three days after transfection. As can be seen from FIG. 16B, both approaches led to a sufficient cell viability.

FIG. 17 shows the graphic representation of the results of studies in liver cancer cell line HLF, wherein the transfection efficiency of conjugated laser-ablated gold nanoparticles has been compared to the efficiency of comparative chemically synthesized gold nanoparticles. The approach was identical to the approach described according to FIG. 15 and FIG. 16, with the exception that linear PEI with a molecular mass of 10 kDa has been used instead of 25 kDa linear PEI. FIG. 17A shows the percentage of GFP positive cells three days after transfection. It can be seen that conjugated gold nanoparticles according to the present invention are linked with significantly higher transfection efficiency when compared to the chemically synthesized gold nanoparticles. In particular, the conjugated gold nanoparticles according to the present invention led to 18.95% to 47.15% GFP positive cells, wherein the comparative particles led only to 2.35% to 9.75% GFP positive cells. Furthermore, the cell viability has been analyzed on the basis of the determination of the percentage of non-apoptotic cells three days after transfection. The results are depicted in FIG. 17B. It can be seen that the conjugated gold nanoparticles according to the present invention are linked with a sufficient viability when conjugation is performed with 9 μg transfection reagent and 1.5 μg or 3 μg nucleic acid molecules per well. The higher amount of polyethylenimine induced more apoptosis. Furthermore, the laser-ablated particles are linked with a lower toxicity than the chemically synthesized nanoparticles, when used with 9 μg PEI.

FIG. 18 shows the graphic representation of the result of studies in non-liver cell line HT1080 in order to analyze the gene transfer efficiency of conjugated laser-ablated gold nanoparticles in comparison to non-inventive chemically synthesized particles. The approach was identical to the approach described in connection with FIG. 17, with the exception of the cell type. The conjugated gold nanoparticles obtained by laser-ablation led to constant transfection rates with 32.65% to 39.6% GFP positive cells. With respect to the chemically synthesized gold nanoparticles, the percentage of GFP positive cells was significantly lower, namely in the range from 3.15% to 32.68%. Overall, the conjugated gold nanoparticles obtained by laser-ablation are linked with higher transfection efficiency. Furthermore, the cell viability has been determined on the basis of the percentage of apoptotic cells three days after transfection. The result are depicted in FIG. 18B. On the basis of a comparison of the results, it can be seen that the laser-ablated gold nanoparticles are linked with significantly lower toxicity in comparison to the chemically synthesized gold nanoparticles.

FIG. 19 shows an image obtained by fluorescence in situ hybridization (FISH), wherein the episomal persistence of the DNA vector pEPI-SM-S (cf. FIG. 3B) with the shortened S/MAR-variant (SEQ ID NO. 20) for a long-term expression of GFP in the liver cancer cell line HLE has been analyzed. For this purpose, the HLE cells have been transfected with conjugated gold nanoparticles, wherein conjugation was performed with branched polyethylenimine with a molecular weight of 25 kDa and vector pEPI-SM-S. In order to confirm that the transfected vector comprising a S/MAR element persists in cells episomally, the HLE cells have been transfected as described before. Subsequent to transfection and cultivation, FISH analysis has been performed. After ten weeks of cultivation with an initial neomycin selection for two weeks, the cells were arrested in metaphases with colcemid and FISH analysis was performed with a biotin-labeled probe for detection of the GFP cDNA. In this context, several GFP signals were detected (cf. small white dots as shown in FIG. 19). As cells arrested in metaphases were dropped onto slides, some of the DNA vectors that were episomally associated with the chromosomes detached from the chromosomes, so that either no or a single signal separated from the chromosome can be detected. Evenly distributed signals on the chromosomes and/or chromatids are an indicator for the integration of the vector. Most of the chromosomes showed only one signal; only one chromosome showed integrated vectors. Therefore, the majority of the DNA comprising a S/MAR-element persisted episomally, despite continuous divisions of the fast growing HLE cells. The low risk of integration of the vector into the genome leads to an improved safety with respect to the use of the conjugated gold nanoparticles in gene therapy.

FIG. 20 shows the graphic representation of studies in fibrosarcoma cells HT1080 and liver cancer cell line HLF, wherein the factor level in cells transfected with pEPI1-SM-S (cf. FIG. 3B), pEFi1-F9co (not shown, identical to pEFi1FPco with the exception that the nucleic acid sequence codes for factor FIX wt), pEFi1-F9Pco (cf. FIG. 3C) has been analyzed. Transfection was performed with conjugated laser-ablated gold nanoparticles, comprising either Transporter5™ (linear PEI, available from Polysciences Europe GmbH, Hirschberg an der Bergstraße, DE) or linear polyethylenimine with a molecular weight of 10 kDa as the transfection reagent. In order to compare the amount of active factor level of coagulation factor FIX in the cell culture supernatant, HT1080 and HLF cells have been transfected with conjugated gold nanoparticles comprising 30 μg laser-ablated gold nanoparticles, 18 μg of the respective transfection reagent and 6 μg DNA (amounts per well). The vector pEPI-SM-S was used in this context as the negative control. For transfection, the conjugated gold nanoparticles were added to the cells (300,000 cells/well in a 6-well format). Cell culture medium was exchanged 4 and 24 hours after transfection and cells were kept in culture for three additional days. Cell culture supernatants were collected to determine the FIX activity by measuring changes in optical density with a turbidimetric method using an ACL Top 500 (Werfen, Kirchheim near Munich, DE). Both cell types transfected with the vector pEFi1-F9co were able to secrete factor FIX into the medium. Higher factor FIX activity was achieved in HT1080 cells. Furthermore, the vector pEFi1-F9Pco, comprising the factor FIX gene with padua mutation, led to significantly higher factor levels than the vector with FIX gene without mutation. Even though factor level in HLF cells was relatively low, with respect to a therapeutic approach it must be pointed out that already low percentages of factor activity are sufficient in order to compensate the negative effect or the phenotype of hemophilia. Against this background, also a low factor activity as achieved in HLF cells could be sufficient with regard to a therapeutic effect in the treatment of hemophilia.

FIG. 21 shows the graphic representation of the results of studies in primary rat hepatocytes, wherein the gene transfer efficiencies and the active factor level of coagulation factor FIX in the cell culture supernatant after transfection with different FIX or GFP in coding vectors have been analyzed. For this purpose, conjugated gold nanoparticles obtained by laser-ablation have been used for transfection. The conjugated gold nanoparticles used for this purpose were based on 30 μg gold nanoparticles and 18 μg transfection reagent (per well with 500,000 cells). In this context, Transporter5™ (Polysciences Europe GmbH, Hirschberg an der Bergstraße, DE) or linear polyethylenimine with a molecular weight of 25 kDa have been used. The nucleic acid molecules have been used in amounts of 3, 6 or 18 μg. For this approach the following vectors were employed: pEGFPC1 (coding sequence for eGFP under the control of a CMV promoter), pCDNA3F9Pco (coding sequence for FIX padua under the control of the CMV promoter), pEFi1EG (coding sequence for eGFP under the control of a promoter derived from human elongation factor-1 alpha, in particular according to SEQ ID NO. 2), pEFi43EG (coding sequence for eGFP under a promoter derived from human elongation factor-1 alpha, in particular according to SEQ ID NO. 3), pEFi43F9Pco (coding sequence for FIX padua under the control of a promoter derived from human elongation factor-1 alpha, in particular according to SEQ ID NO. 3). On the basis of GFP as the marker gene, the transfection efficiency was analyzed by flow cytometry. On the basis of cells transfected with the vectors containing a coding sequence for coagulation factor FIX padua, the factor level and factor activity in the culture supernatants of the cells have been determined. For the purpose of transfection, conjugated gold nanoparticles according to the present invention on the basis of 30 μg gold nanoparticles, 9 μg or 18 μg transfection reagent and 3 μg, 6 μg or 18 μg (amounts per well) have been added to the cells (500,000 cells/well in a 6-well format). Furthermore, a comparison was performed for each approach also without gold nanoparticles (negative control). Cell culture medium was exchanged 4 and 24 hours after transfection and cells were incubated for additional three days. Subsequently, supernatants were collected for FIX activity analysis and GFP-transfected cells were analyzed for GFP expression by flow cytometry.

FIG. 21A shows, that the conjugated laser-ablated gold nanoparticles have the ability to transfect primary rat hepatocytes, i.e. mammalian liver cells. Furthermore, the mean fluorescence intensity (MFI) of the GFP in the cell has been determined (FIG. 21B). The determination of the mean fluorescence intensity also confirms that the conjugated gold nanoparticles obtained by laser ablation according to the present invention are suitable for the transfection of liver cells, in particular hepatocytes. FIG. 21C shows the results of an analysis of the cell viability on the basis of the determination of the percentage of non-apoptotic cells three days after transfection. As can be seen from FIG. 21C, all approaches are linked with a sufficient cell viability. FIG. 21D shows the results of the determination of the factor level of coagulation factor FIX. From the results obtained on the basis of the determination of the active factor level, it is evident that the use of conjugated laser-ablated gold nanoparticles leads to a significantly improved production of coagulation factor FIX in liver cells. In this context, with the vector pCDNA3F9Pco an active factor level of 48.5% was achieved, wherein transfection with the vector pEFi43F9Pco led to 13.4% active factor level, which is still promising approach with respect to the realization of a therapeutic concept, in particular gene therapy, for the treatment of hemophilia.

FIG. 22 shows the graphic representation of the result of studies performed in liver cancer cell line HLF, wherein the transfection efficiency of conjugated gold nanoparticles according to the present invention has been analyzed. In this context, the production of the conjugated gold nanoparticles has been performed according to a particularly preferred embodiment of the method according to the present invention, wherein the conjugation of the gold nanoparticles with the transfection reagent has been performed simultaneously to generating the gold nanoparticles as such by pulsed laser ablation in liquid. In this context, the buffer which has been used for pulsed-laser ablation in liquid contained different concentrations of branched polyethylenimine with a molecular mass of 25 kDa, namely concentrations of 10 μg/ml, 25 μg/ml, 50 μg/ml or 100 μg/ml. With respect to the nucleic acid molecules, the gold nanoparticles have been conjugated with the vector pEPI-SM-S according to FIG. 3B.

With respect to the preparation of the conjugated gold nanoparticles, gold foils have been used as gold target for the generation of gold nanoparticles with an average particle diameter of 5 nm by pulsed laser ablation in liquid (PLAL). PLAL has performed in solutions containing the above-mentioned concentrations of branched polyethylenimine. The different concentrations of the transfection agent were chosen to define optimal properties concerning the stability of the conjugated gold nanoparticles, gene transfer and toxicity effects. The gold nanoparticles conjugated or complexed with the transfection agent comprised after laser ablation an increased hydrodynamic diameter in the range of 14 to 22 nm, determined by dynamic light scattering. For the purpose of transfection, conjugated gold nanoparticles were prepared by adding 2 μg, 6 μg and 9 μg of nucleic acid molecules to 30 μg gold nanoparticles generated and complexed with the transfection reagent by pulsed laser ablation in liquid. The mixture was added to the cells, wherein each well of a 6-well plate contained 300,000 cells. After 4 hours and 24 hours, the cell culture medium was exchanged and cells were kept in culture for additional three days. Thereafter, HLF cells were collected and analyzed by flow cytometry. FIG. 22A shows the percentage of GFP positive cells three days after transfection. A particularly efficient gene transfer was achieved with gold nanoparticles that were generated in solutions with 25 μg/ml or 50 μg/ml of the polyethylenimine. Furthermore, the mean fluorescence index of the GFP positive cells was determined. In this context, transfection with gold nanoparticles generated in solutions with higher polyethylenimine concentrations led to higher mean fluorescence intensities in GFP positive cells, as depicted in FIG. 22B. FIG. 22C shows the result of the analysis of the cell viability on the basis of the determination of the percentage of non-apoptotic cells three days after transfection. As can be seen from FIG. 22C, all approaches led to a sufficient cell viability. Nevertheless, the use of gold nanoparticles generated in higher concentrations of polyethylenimine for transfection was associated with slightly higher toxicity effects.

FIG. 23 shows the graphic representation of the results of studies in cells of the HT1080 fibrosarcoma cell line. The respective approach was identical to the approach described in connection with FIG. 22, except for the cell type. FIG. 23A relates to the percentage of GFP positive cells three days after transfection. It can be seen that particularly good results, i.e. the most efficient gene transfer, are achieved with conjugated gold nanoparticles that were generated in solutions containing 50 μg/ml or 100 μg/ml polyethylenimine. Furthermore, the mean fluorescence intensity (MFI) values have been determined as indicator for the amount of DNA transported into cells that became GFP. The respective results are depicted in FIG. 23B. In this context, it can be seen that transfection with gold nanoparticles generated in solutions with higher polyethylenimine concentrations led to higher MFI values in GFP positive cells. Nevertheless, cells transfected with gold nanoparticles generated in the highest polyethylenimine concentration (100 μg/ml ) showed a decreasing MFI level again. Against this background, overall, a polyethylenimine concentration of 50 μg/ml in the solution for laser ablation in liquid seems to be favorable. FIG. 23C contains the results with respect to the analysis of the cell viability on the basis of the determination of the percentage of non-apoptotic cells three days after transfection. It can be seen that all approaches led to sufficient cell viability.

FIG. 24 shows the graphic representation of the results of studies performed in liver cancer cell line HLF, wherein the gene transfer efficiency based on GFP transgene expression mediated by a particularly preferred embodiment of conjugated gold nanoparticles according to the present invention has been analyzed. The conjugated gold nanoparticles were based on laser-ablated gold nanoparticles with an average diameter of 5 nm, conjugated on the basis of a layer-by-layer assembly with an inner polyethylenimine layer comprising Transporter5™ (linear PEI, Polysciences Europe GmbH, Hirschberg an der Bergstraße, DE) and an outer layer on the basis of jetPEI®-hepatocyte (galactose-conjugated polyethylenimine, PolyPlus Inc., Illkirch, FR) or Transporter5™. In particular, on the basis of this approach it was analyzed whether a second layer of transfection reagent leads to higher transfection and/or gene transfer efficiencies. For this purpose, laser-ablated gold nanoparticles were complexed with Transporter5™ as transfection reagent and nucleic acid molecules on the basis of the vector pEPI-SM-S (cf. FIG. 3B). After complexing, a purification using Vivaspin© columns has been performed. The purified complex of gold nanoparticles, the first transfection reagent and nucleic acid molecules was covered and/or conjugated with a second layer of polyethylenimine. For the second layer, either a galactose-conjugated polyethylenimine (jetPEI®-hepatocyte) or Transporter5™ have been employed. With respect to the preparation of conjugated gold nanoparticles with a layer-by-layer assembly, reference is also made to FIG. 1B.

In particular, a stable amount of gold nanoparticles of 30 μg, Transporter5™ and nucleic acid molecules (each 3 μg) were transfected with different amounts of the second transfection reagent (up to 9 μg). For this purpose, 3 μg of nucleic acid molecules were mixed with 30 μg laser-ablated gold nanoparticles having a size of 5 nm that were covered before with 9 μg transfection reagent on the basis of Transporter5™. Th After adding the nucleic acid molecules, a second layer of Transporter5™ or jetPEI®-hepatocyte was applied and added to the cells (300,000 cells/well in a 6-well format). Cell medium was exchanged 4 and 24 hours after transfection. Cells were kept in culture for two additional days and then analyzed by flow cytometry to determine the percentage of GFP expressing cells.

FIG. 24A shows the percentage of GFP positive cells three days after transfection. It can be seen that the transfection rates achieved in HLF cells by using 1.3 or 9 μg jetPEI®-hepatocyte as the second layer of the layer-by-layer assembly were always higher compared to the use Transporter5™ as second transfection reagent (45%, 57%, 62% compared to 40%, 56%, 58%). Furthermore, it is noted that higher amounts of polyethylenimine led to larger amounts of GFP positive cells, independently from the polyethylenimine variant. Overall, all tested approaches led to sufficient gene transfer efficiency. Furthermore, the mean fluorescence intensity (MFI) has been determined. The results are depicted in FIG. 24B and confirm the results shown in FIG. 24A. Furthermore, FIG. 24C shows the result of an analysis of the cell viability on the basis of the determination of the percentage of non-apoptotic cells three days after transfection. It can be seen that higher amounts of polyethylenimine led to a higher toxicity and induced more apoptosis. Nevertheless, conjugated gold nanoparticles according to a preferred embodiment of the present invention on the basis of a layer-by-layer assembly are linked with sufficient cell viability.

FIG. 25 shows the graphic representation of the results of studies performed in non-liver cell line HT1080 in order to analyze the gene transfer efficiencies based on conjugated gold nanoparticles comprising a layer-by-layer assembly. The approach was identical to the approach described in connection with FIG. 24, except for the cell type.

FIG. 25A shows the percentage of GFP positive cells three days after transfection. It can be seen that both variants of polyethylenimine in the outer PEI layer led to similar results concerning the amount of GFP positive cells. The highest amount of GFP positive cells was achieved with the highest amount of polyethylenimine complexed with the gold nanoparticles. Furthermore, the mean fluorescence intensities (MFIs) have been determined as indicator for the amount of DNA transported into cells that became GFP positive. The respective results are depicted in FIG. 25B. In this context, all approaches led to a sufficient amount of transferred DNA. Particularly high MFI levels were obtained with conjugated gold nanoparticles comprising Transporter5™ as outer layer. FIG. 25C shows the result of an analysis of the cell viability on the basis of the determination of the percentage of non-apoptotic cells three days after transfection. It can be seen that higher amounts of polyethylenimine are linked with a higher percentage of apoptotic cells. Against this background, the use of smaller amounts of polyethylenimine in the outer layer seems to be more favorable.

FIG. 26 shows the graphic representation of the results of studies performed in liver cancer cell line HLF, wherein cells have been transfected with vectors comprising the GFP-gene under the control of either the hAAT-promoter (vector peAATEG according to FIG. 3M) or the SERPINA1-promoter (vector peSEREG according to FIG. 3D). In this context, two different DNA concentrations of 3 μg or 6 μg were employed. As transfection reagent, 18 μg of Transporter5™ (Polysciences Europe GmbH, Hirschberg an der Bergstraße, DE) have been used. For the purpose of transfection, 3 μg or 6 μg of the respective vector were mixed with 18 μg transfection reagent and added to the cells (300,000 cells/well in a 6-well format). Cell culture medium was exchanged 4 and 24 hours after transfection. Cells were kept in cultures for two additional days and then analyzed by flow cytometry to determine the percentage of GFP expressing cells.

FIG. 26A shows the percentage of GFP positive cells two days after transfection. For both concentrations of transfected nucleic acid molecules, significantly higher proportions of GFP positive cells were detected for the construct with the hAAT-promoter compared to the vector with the SERPINA1-promoter. Furthermore, the mean fluorescence intensity level (MFI) has been determined. Also with respect to the MFI level, the hAAT-promoter led to a higher expression level of the marker gene GFP in comparison to the SERPINA1-promoter (cf. FIG. 26B). On the basis of the results with respect to the percentage of GFP positive cells and the MFI values, it can be assumed that particularly the hAAT promoter directs an improved expression of the coding sequence. Furthermore, FIG. 26C shows the result of an analysis of the cell viability on the basis of the determination of the percentage of non-apoptotic cells two days after transfection. Overall, sufficient cell viability was achieved on the basis of both approaches. Nevertheless, the combination of 18 μg Transporter5™ as the transfection reagent and an amount 3 μg DNA showed slightly higher toxicity effects.

FIG. 27 shows the graphic representation of the results of studies performed in non-liver cells HT1080. In this context, the transgene expression under the promoters hAAT and SERPINA1 have been analyzed. The approach was identical to the approach according to FIG. 26, with the exception of the cell type.

FIG. 27A shows the percentage of GFP positive cells three days after transfection. It can be seen that both concentrations of plasmids transfected and both promoters showed similar transfection efficiency. Furthermore, the mean fluorescence intensity values (MFIs) have been determined. The respective results are depicted in FIG. 27B. Higher MFI values were measured for the expression of GFP under the control of the SERPINA1-promoter. FIG. 27C shows the results of the analysis of the cell viability on the basis of the determination of the percentage of a non-apoptotic cells three days after transfection. In this context, no toxicity effects were observed with respect to both approaches.

FIG. 28 shows images obtained by TEM-analyses of unconjugated gold nanoparticles (FIG. 28A) and PEI-conjugated gold nanoparticles (FIG. 28B). In this context, FIG. 28 focuses on a comparison of laser-ablated gold nanoparticles (FIGS. 28A, B, bottom row) and chemically synthesized gold nanoparticles (FIGS. 28A, B, upper row).

It can be seen from FIG. 28A that both methods lead to naked, unconjugated gold nanoparticles with an even particle distribution (FIG. 28A). Despite the even particle distribution, the surface of unconjugated gold nanoparticles obtained by chemical synthesis needs to be stabilized in a solution comprising sodium citrate. Stabilizing agents can lower the compatibility of the gold nanoparticles when used in medical applications. In contrast to this, can be diluted in phosphate buffer without any of further stabilization.

FIG. 28B shows both types of gold nanoparticles after conjugation with 25 kDa polyethylenimine. The transfection agent of the chemically synthesized gold nanoparticles was covalently bound to the surface of the nanoparticles by thiol groups. The binding of the transfection reagent to the laser-ablated gold nanoparticles was based on electrostatic interactions. The TEM images (cf. FIG. 28B) revealed that the size distribution of the conjugated particles based on chemically synthesized gold nanoparticles varied in wide ranges (cf. FIG. 28B, upper row). In contrast to this, gold nanoparticles conjugated with polyethylenimine on the basis of laser-ablated gold nanoparticles comprise an evenly size distribution, which is particularly advantageous with respect to the use in gene therapy (cf. FIG. 28B, bottom row).

The following working examples better illustrate the subject-matter of the present invention, and they should not be considered limiting the application.

### WORKING EXAMPLES

In order to illustrate the present invention, in particular the underlying principles and advantages, various transfection studies have been performed, as delineated in the following.
1. General Experimental Procedures

Pulsed Laser Ablation in Liquid (PLAL)

The preparation of ligand-free (naked) gold nanoparticles has been performed with the method of pulsed laser ablation in liquid. For this purpose, a picosecond laser (available from Ekspla Atlantic, Vilnius, Lithuania) has been used. The laser ablation has been performed in phosphate buffered saline or sodium phosphate buffer (NaPP) as liquid with a pulse duration of 8 to 15 ps, up to 160 μJ pulse energy, a repetition rate of 80 to 150 kHz and a wavelength of 1,064 nm. Furthermore, the ablation was carried out in a 30 ml batch chamber for 10 min duration. As gold target, gold foil with a thickness of about 500 μm has been used.

Gold nanoparticles with a size of 10 nm or less have been obtained by using 600 μM sodium phosphate buffer (NaPP) as the liquid for laser ablation. In order to harvest particles with an average particle diameter of 10 nm or less, particles of larger size have been separated by ultracentrifugation (30,000×g, 17 min, 7° C.). While larger particles have been pelleted and discarded, particles of smaller size smaller than 10 nm remained in the supernatant and have been kept for further processing, i.e. conjugation with transfection reagent and nucleic acid molecules.

Larger particles with a size of 10 nm or more, in particular 40 to 60 nm, have been synthesized in purified water or phosphate buffered saline as liquid. The particles with a size of 10 nm or more, in particular 40 to 60 nm, have been obtained by incubating the particles after laser ablation for riping for at least 24 hours. After the incubation time the particles have been centrifuged (for example at 10,000 rpm, 70 min, 7° C.) to remove smaller particles. The supernatant containing smaller particles has been discarded while the larger particles in the pellet were re-suspended in suitable medium, for example purified water or a non-toxic buffer.

The unconjugated “naked” particles obtained by pulsed laser ablation in liquid have been conjugated with polyethylenimine and nucleic acid molecules, as delineated hereinafter.

Conjugation with polyethylenimine During Pulsed Laser Ablation in Liquid

According to a preferred embodiment of the method according to the present invention, conjugation of the gold nanoparticles with the transfection agent was performed simultaneously with pulsed laser ablation in liquid. In this context, pulsed laser ablation in liquid was performed according to the protocol as given above. Additionally, polyethylenimine was added to the liquid in the desired concentrations, in particular 10 μg/ml, 25 μg/ml, 50 μg/ml or 100 μ/ml, based on the liquid.

Preparation of Branched and Linear Polyethylenimine (PEI)

Branched PEI (Sigma Aldrich, 25 kDa) is a highly viscous solution. It was weighed, dissolved in PBS and adjusted to a 100 mg/ml stock solution. For use, stock solution was diluted to 1 mg/ml, filtered through a 0.22 μm membrane and stored at 4° C. The 10 kDa and 25 kDa linear PEIs (Polysciences Inc., Warrington, Pa., USA) were bought as powder and dissolved in water before using. To this end, the PEI was mixed with UltraPure distilled water at a concentration of 1 mg/ml and then heated to 80° C. until the solution was clear. The PEI solution was then cooled to room temperature and the pH value was adjusted to 7.0 using HCl. The PEI solution was then sterile filtered through a 0.22 μm membrane filter and stored at 4° C. The molecular weight of PEI has been determined by means of gel permeation chromatography or according to DIN 55672-3: 2016-03, respectively. Commercially available PEI variants jetPEI® and jetPEI®-hepatocyte (Polyplus Inc., Illkirch, FR) as well as Transporter5™ (Polysciences Europe GmbH, Hirschberg an der Bergstraße, DE) are delivered in a ready-to-use state.

Conjugation of Gold Nanoparticles with polyethylenimine by Admixing

In order to conjugate gold nanoparticles with polyethylenimine by admixing, the gold nanoparticles obtained by laser ablation have been incubated with the transfection reagent one day before transfection and dialyzed against ddH₂O with a 50 kDa molecular weight cut off. The gold nanoparticles were diluted with ddH₂O to a concentration of 160 μg/ml before using.

Conjugation of PEI-Conjugated Gold Nanoparticles with Nucleic Acid Molecules

Gold nanoparticles conjugated with transfection agents on the basis of polyethylenimine have been further conjugated with nucleic acid molecules by adding nucleic acid molecules in the desired amounts to the PEI-conjugated gold nanoparticles. In particular, further conjugation of the gold nanoparticles with nucleic acid molecules is performed immediately before transfection. In this context, reference can also be made to the further explanations regarding the transfection as such.

Vectors Designed for Further Expression Studies

The vectors, in particular the vectors as shown according to FIG. 3, have been generated by using standard cloning techniques. In particular, preparation of purified plasmid DNA in high quantities was performed with the NucleoBond® Xtra Maxi Kit (Macherey-Nagel, Düren, Germany) according to the manufacturer's instructions after transformation of chemically competent One Shot® TOP10 E. coli (Thermo Fisher Scientific, Waltham, Mass., US). In particular, reference is made to FIG. 3A to 3M, showing the respective maps of the vectors used for the expression studies.

Cell Cultures

For transfection analyses, the liver cancer cell lines HLF and HLE have been used. Both cell lines originate from human hepatocellular carcinoma. The HLF and HLE cells derived from the same patient have been obtained form the Riken Tissue bank in Japan. Furthermore, the cell line HT1080 has been used in order to analyze the transfection and expression in non-liver tissue, in particular fibroblasts. The cell line HT1080 is a human fibrosarcoma cell line (DMSZ, Braunschweig, Germany). In addition, transfection experiments have been transformed in rat hepatocytes. The cells were grown in Dulbecco's Eagle's Medium (DMEM) with 4,6 mM glucose and 2 mM GlutaMAX™ supplement with 10 wt.-% fetal bovine serum, 100 U/ml penicillin and 100 μg/ml streptomycin. For antibiotic selection with the neomycin analogue geneticin (G418), the medium was supplemented with 1 mg/ml geneticin (commercially available from Gibco BRL, Thermo Fisher Scientific). All cells are adherent and form monolayers in culture; they have been split two to three times a week. For splitting, the cultures were washed with a solution on the basis of phosphate buffered saline (PBS, commercially available from Gibco BRL, Thermo Fisher Scientific) and incubated with Trypsin-EDTA until the monolayer dissociated. Cells were then transferred into new cell culture dishes based to their proliferation rate. Cells were grown at 37° C. in an atmosphere with 5 vol.-% CO₂.

General Transfection Protocol

The transfection as such has been performed according to standard protocols. In particular, for transfection 200,000, 300,000 or 500,000 cells were seeded in 6-well tissue-culture plates. Cell counting of the different cell lines has been performed by using a Neubauer counting chamber. At the next day, cells were transfected with vector DNA using different transfection reagents. In this context, cells were cultured in 1 ml standard culture medium with the transfection reagent. 6 hours after transfection, standard medium was added to the cell culture wells. 24 hours after transfection, the medium was exchanged. After two or three days, GFP-expression was determined via Fluorescence-activated cell sorting (FACS) analysis.

Transfection with Polyethylenimine (Without Gold Nanoparticles)

For transfection with PEI as transfection reagent, DNA and PEI were separately diluted in 100 μl 150 mM NaCl. The PEI solution was then added to the DNA solution. The PEI/DNA solution was mixed, incubated for 15 minutes at room temperature and then added to the cells.

Transfection with Conjugated Gold Nanoparticles

HLF cells, HT1080 cells and rat hepatocytes were transfected with conjugated gold nanoparticles according to the present invention. The unconjugated gold nanoparticles had an average particle diameter of either 5 nm or 50 nm, determined by analytical disc centrifugation and transmission electron microscopy (TEM). Furthermore, different variants of PEI have been used, namely 25 kDa branched PEI (for example available from nanoComposix Europe, Prague, CZ), 25 kDa linear PEI (for example available from Nanopartz Inc, Loveland, Calif.), Transporter5™ (Polysciences Europe GmbH, Hirschberg an der Bergstraße, DE) and jetPEI®/jetPEI®-Hepatocyte (Polyplus Inc., Illkirch, FR). For the purpose of transfection with conjugated gold nanoparticles according to the present invention, the naked or unconjugated gold nanoparticles have been pre-incubated with the transfection reagent one day before transfection and dialyzed against ddH₂O with a 50 kDa molecular weight cut off. The PEI-conjugated gold nanoparticles were diluted with ddH₂O to a concentration of 160 μg/ml before using. Afterwards, the complexes of gold nanoparticles and polyethylenimine were further conjugated with the DNA by incubating them with the nucleic acid molecules for 2 to 5 minutes before adding to the cells for the purpose of transfection.

Fluorescence-Activated Cell Sorting (FACS)

FACS analyses were conducted to determine the number of GFP-expressing cells, as well as the mean fluorescent intensity (MFI) and the amount of non-apoptotic cells three days after transfection. In this context, cells were washed once with 2 ml phosphate buffered saline (PBS). Afterwards the cells were trypsinized with 0.5 ml Trypsin-EDTA (0.05 wt.-% Trypsin, 0.02 wt.-% EDTA) and the reaction was stopped by adding cell culture medium. The detached cells were transferred into a FACS tube and centrifuged for 5 min at 1,200 rpm. The supernatant was then removed and the cell pellet dissolved using PBS containing 2 wt.-% fetal calf serum (FCS) and 4′,6-diamidino-2-phenylindole (DAPI). For every FACS analysis a sample without DAPI-staining was furthermore analyzed. Data analysis was conducted using BD FACSDiva™ as software.

Factor Level Measurement

In order to determine the factor level, 24 hours after transfection, the cell culture medium was removed and the cells were cultured in 1 ml medium. After another 24 hours, the cell culture supernatant was collected and immediately frozen at −80° C. until factor level measurement was performed. During factor level measurement the amount of time, which is required for a plasma sample to clot, is recorded. Coagulation endpoints have been assessed by measuring changes in optical density with a turbidimetric method. All measurements were conducted using an ACL Top 500 (Werfen GmbH, Kirchheim near Munich, Del.).
2. Results of the Cell Culture Studies

With respect to the provision of conjugated gold nanoparticles for the use in an improved genetic approach for the treatment of monogenetic disorders, in particular haemophilia, studies with different malignant cell types and non-malignant rat hepatocytes have been performed. The results of the studies performed serve as a basis for the preparation of conjugated gold nanoparticles for the use in gene therapy and/or a nanoparticle-based delivery system for the use in gene therapy of monogenetic disorders.

Influence of the S/MAR Element on Transfection and Expression Efficiency

In order to establish an optimal S/MAR variant with respect to a long-term expression—i. e. episomal persistence—of the coding sequence in the target cells, in particular the liver or fibrous tissue, the long-term expression of GFP under different S/MAR variants in various cell types transfected with the afore described test vectors pEPI1-SM-L and pEPI1-SM-S (cf. FIGS. 3A and 3B) was recorded.

Transfection of Cell Lines

In order to test the influence of different S/MAR variants on the episomal persistence of nucleic acid molecules, liver cancer cells of the human hepatoma cell line HLF have been transfected with the afore-described vectors pEPI1-SM-S (FIG. 3B) and pEPI1-SM-L (FIG. 3A) by using conjugated laser-ablated gold nanoparticles. For this purpose, conjugated gold nanoparticles have been prepared by pre-incubating laser-ablated gold nanoparticles with 25 kDa branched PEI, followed by dialyzing and diluting the particles. The PEI-coated gold nanoparticles have been further conjugated with the nucleic acid molecules and used for transfection of the cells. Per assay (i.e. per 300,000 cells per well of a 6-well format), 6 μg DNA, 18 μg branched PEI and 30 μg gold nanoparticles have been used for transfection.

Test Procedure

The expression of GFP in the transfected cells was measured as an indicator for episomal persistence 24 hours after transfection. Afterwards, GFP expression in the cells was measured weekly. Since the malignant cell lines used for the test series are—in contrast to healthy liver cells, in particular hepatocytes, and healthy fibrous tissue cells—fast dividing cells, the test series were performed under short-term selection conditions on the basis of geneticin (G418) present for 2 weeks and long-term selection conditions on the basis of geneticin (G418) present over the whole observation period. In order to measure the expression of GFP, cells were harvested and analyzed by flow cytometry. In this context, the percentage of cells expressing GFP was determined. Furthermore, the MFI has been determined.

Results

The results of the transfection experiments regarding the influence of different variants of the S/MAR elements on episomal persistence are graphically depicted in FIG. 5A (short-term selection) and FIG. 5B (long-term selection). As can be seen from FIG. 5A, both variants of the S/MAR element led to a long-term expression of eGFP in the transfected cells after short-term selection. A higher percentage of eGFP positive cells has been surprisingly achieved with the shortened variant of the S/MAR element. Furthermore, as can be seen from FIG. 5B, both variants of the S/MAR element led to a long-term expression of eGFP in the transfected cells under long-term selection with G418.

Influence of the Particle Size of the Gold Nanoparticles on Transfection Efficiency

Furthermore, the influence of the size, i.e. the average particle diameter, of the “naked” laser-ablated gold nanoparticles on the transfection efficiency of conjugated gold nanoparticles has been investigated. For this purpose, liver cancer cell lines HLF and HepG2 have been conjugated with conjugated gold nanoparticles on the basis of laser-ablated particles with a size of 5 nm or 50 nm, respectively. As transfection reagent, the particles comprised 25 kDa branched PEI and as nucleic acid molecules the vector pEPI-SM-S (cf. FIG. 3B) or pEPI-F8-SM-S (map not shown, comprises a coding sequence for a fusion of factor FVIII and eGFP under transcriptional control of the CMV promoter and the shortened variant of the S/MAR element).

Transfection of Cell Lines

With respect to transfection, 200,000 cells per well in a 6-well format have been transfected with conjugated gold nanoparticles on the basis of 30 μg gold nanoparticles with 18 μg of 25 kDa branched PEI and 20 μg nucleic acid molecules. As negative control, cells have been transfected without gold nanoparticles, wherein the same amount nucleic acid molecules and polyethylenimine has been used.

Test Procedure

Cells were analyzed for GFP expression three days after transfection by flow cytometry. In this context, the percentage of cells expressing GFP was determined.

Results

FIG. 6A shows the result of the analysis of the eGFP expression in HLF cells. It can be seen that particularly good results are achieved with gold nanoparticles with a diameter of 5 nm in the unconjugated state. FIG. 6B shows the result of the analysis of the eGFP expression in HepG2 cells. The transfection efficiency of HepG2 cells was also higher when particles had a diameter of 5 nm. Overall, on the basis of the smaller particles a higher transfection efficiency is achieved.

Influence of the Weight Related Ratio of DNA to polyethylenimine

According to the studies performed by applicant, the influence of the weight related ratio of the DNA to polyethylenimine in conjugated laser-ablated gold nanoparticles has been investigated. In this context, different variants of polyethylenimine (branched PEI and linear PEI, both with a molecular mass of 25 kDa) have been tested. Furthermore, different weight related ratios of transfection reagent to nucleic acid molecules of 1:1.1, 3:1, 6:1 and about 12:1 have been tested. The conjugated gold nanoparticles used for this test series comprised the vector pEPI-SM-S (cf. FIG. 3B) as nucleic acid molecules.

Transfection of Cell Lines

200,000 cells (HLF or HT1080) per well of a 6-well plate have been transfected with conjugated gold nanoparticles on the basis of 30 μg laser-ablated gold nanoparticles with an average particle diameter of 5 nm, nucleic acid molecules in amounts of 0.7 μg, 1.5 μg, 3 μg or 10 μg, respectively, and polyethylenimine (either branched or linear PEI with a molecular mass of 25 kDa) in an amount of 9 μg.

Test Procedure

Cells were analyzed for GFP expression three days after transfection by flow cytometry. In this context, the percentage of cells expressing GFP was determined (FIGS. 9A and 10A). Furthermore, the mean fluorescence intensity (MFI) levels have been determined as an indicator for the amount of the transferred nucleic acid molecules (FIGS. 9B and 10B). In order to analyze the toxicity of the conjugated gold nanoparticles, cell viability has been determined on the basis of the percentage of non-apoptotic cells (FIGS. 9C and 10C).

Results

FIGS. 9A to 9C contain the results with respect to the HLF cells. In this context, the highest GFP expression levels were observed with a weight related ratio of polyethylenimine to nucleic acid molecules of 3:1 (cf. FIG. 9A). Furthermore, as can be seen from FIG. 9C, the transfection with all variants of polyethylenimine in different weight related ratios to the nucleic acid molecules was linked with a sufficient cell viability.

FIGS. 10A to 10C show the results with respect to the HT1080 cells. It can be seen from FIG. 10A that both variants of polyethylenimine achieved the highest percentage of GFP positive cells at different ratios of polyethylenimine to nucleic acid molecules. With respect to the branched polyethylenimine, a ratio of polyethylenimine to nucleic acid molecules of 3:1 led to the highest expression levels of GFP, whereas for the linear polyethylenimine the ratio of about 12:1 seemed to be more favorable. Nevertheless, also with a ratio of 3:1 or 6:1 sufficient results have been achieved. FIG. 10B shows the results of the mean fluorescence intensity (MFI) of the GFP positive cells. In this context it can be seen that for both polyethylenimine variants, higher mean fluorescence intensities correlated with higher amounts of nucleic acid molecules. Furthermore, it can be seen from FIG. 10C that all variants of polyethylenimine as well as all tested amounts of DNA used for transfection led to a sufficient viability of the transfected cells.

Influence of the polyethylenimine Variant

According to the studies performed by applicant, the influence of the polyethylenimine variant in the conjugated gold nanoparticles according to the present invention on the transfection and expression efficiency has been investigated. In this context, different variants of polyethylenimine (25 kDa linear PEI, 10 kDa linear PEI, Transporter5™ and linear jetPEI®) have been tested as transfection reagent in conjugated gold nanoparticles. Furthermore, in this context the conjugated gold nanoparticles were tested with two different quantities of PEI and two different weight related ratios of transfection reagent to nucleic acid molecules.

Transfection Procedure

200,000 cells (HLF or HT1080) per well of a 6-well plate have been transfected with conjugated gold nanoparticles on the basis of 30 μg laser-ablated gold nanoparticles with a particle size of 5 nm, nucleic acid molecules in amounts of 1.5 μg, 3 μg or 6 μg and polyethylenimine (25 kDa linear PEI, 10 kDa linear PEI, Transporter5™ or linear jetPEI®) in an amount of 9 μg or 18 μg.

Test Procedure

Cells were analyzed for GFP expression three days after transfection by flow cytometry. In this context, the percentage of cells expressing GFP was determined (FIGS. 13A and 14A). Furthermore, the mean fluorescence intensity (MFI) levels have been determined as an indicator for the amount of the transferred nucleic acid molecules (FIGS. 13B and 14B). In order to analyze the toxicity of the conjugated gold nanoparticles, cell viability has been determined on the basis of the percentage of non-apoptotic cells (FIGS. 13C and 14C).

Results

With respect to the results of studies in liver cancer cell line HLF, it can be seen from FIG. 13A that the highest amount of GFP expressing cells was achieved with 10 kDA polyethylenimine at a 6:1 ratio of polyethylenimine to nucleic acid molecules. In general, higher quantities of polyethylenimine led to larger GFP positive cell populations. Furthermore, as can be seen from FIG. 13B, the highest MFI values were obtained with Transporter5™ and 10 kDa linear polyethylenimine as the transfection reagents and with the highest concentration of DNA of 6 μg. Furthermore, as can be seen from FIG. 13C, all tested approaches led to a sufficient cell viability, even though higher amounts of polyethylenimine and nucleic acid molecules were associated with slightly more toxicity in HLF cells.

With respect to the results of studies in HT1080 cells, it can be seen from FIG. 14A that the highest amount of GFP expressing cells was achieved with

JetPEI® with a weight related ratio of polyethylenimine to nucleic acid molecules of 3:1. Except for linear polyethylenimine with a molecular mass of 10 kDa, higher amounts of polyethylenimine and nucleic acid molecules resulted in higher GFP expression levels for all tested variants of polyethylenimine. The results are further confirmed by the results of the determination of the mean fluorescence intensity (MFI) of eGFP in the GFP positive cells, which are depicted in FIG. 14B. Additionally, as can be seen from FIG. 14C, except for the linear 10 kDa polyethylenimine, not much toxicity or apoptosis was observed when transfecting HT1080 cells with conjugated gold nanoparticles comprising different PEI variants.

Comparison of Laser-Ablated and Chemically Synthesized Gold Nanoparticles

Furthermore, studies have been performed in order to compare the influence of the use of laser-ablated gold nanoparticles to the use of chemically synthesized nanoparticles on the transfection and expression efficiency. The respective studies have been performed in liver cancer cell line HLF and fibrosarcoma cell line HT1080. In this context, conjugated gold nanoparticles comprising either 10 kDa linear or 25 kDa branched PEI have been used. As nucleic acid molecules, the conjugated gold nanoparticles comprised the vector pEPI-SM-S (cf. FIG. 3B). PEI-conjugated chemically synthesized gold nanoparticles have been obtained from Nanopartz Inc., Loveland, Colo., US and further conjugated with the nucleic acid molecules.

Transfection Procedure 200,000 cells (HLF or HT1080) per well of a 6-well plate have been transfected either with conjugated gold nanoparticles on the basis of laser-ablated gold nanoparticles with an average particle diameter of 5 nm, nucleic acid molecules in amounts of 1.5 μg, 3 μg or 6 μg and polyethylenimine (either 25 kDa linear PEI or 10 kDa linear PEI) in an amount of 9 μg or 18 μg or with chemically synthesized gold nanoparticles comprising 25 kDa linear PEI or 10 kDa linear PEI as transfection reagent and nucleic acid molecules in amounts of 350 ng, 1 μg, 3 μg, 6 μg, 9 μg or 20 μg.

Test Procedure

Cells were analyzed for GFP expression three days after transfection by flow cytometry. In this context, the percentage of cells expressing GFP was determined (FIGS. 15A, 16A, 17A and 18A). In order to analyze the toxicity of the conjugated gold nanoparticles, cell viability has been determined on the basis of the percentage of non-apoptotic cells (FIGS. 15B, 16B, 17B and 18B).

Results

FIG. 15 shows the graphic representation of the results of studies in liver cancer cell line HLF with 25 kDa linear PEI as transfection reagent. It can be seen from FIG. 15A that conjugated gold nanoparticles on the basis of laser-ablated particles comprise a significantly higher transfection efficiency when compared to conjugated gold nanoparticles on the basis of chemically synthesized particles. With the conjugated gold nanoparticles obtained by laser ablation, 16.17% to 35.85% GFP positive cells have been obtained by transfection. In contrast to this, chemically synthesized nanoparticles led only to 0.15% to 0.38% GFP positive cells. Furthermore, as can be seen from FIG. 15B, both approaches led to a sufficient cell viability, even though toxicity of the chemically synthesized gold nanoparticles in HLF cells was slightly lower. Nevertheless, the cell viability achieved with the conjugated gold nanoparticles according to the present invention is still sufficient.

FIG. 16 shows the graphic representation of the results of studies in HT1080 cells with 25 kDa linear PEI as transfection reagent. It can be seen from FIG. 16A that laser-ablated gold nanoparticles in the conjugated gold nanoparticles were largely superior with respect to the transfection efficiency when compared to conjugated gold nanoparticles on the basis of chemically synthesized gold nanoparticles. In this context, on the basis of the conjugated gold nanoparticles according to the present invention, transfection led to 48.13% to 70.91% GFP positive cells. In contrast to this, the chemically synthesized gold nanoparticles resulted in only 0.65% to 3.25% GFP positive cells. Furthermore, as can be seen from FIG. 16B, both approaches led to a sufficient cell viability.

FIG. 17 shows the results of studies in liver cancer cell line HLF with 10 kDa linear PEI as transfection reagent. In this context, it can be seen from FIG. 17A that conjugated gold nanoparticles obtained by laser ablation are linked with a significantly higher transfection efficiency when compared to the comparative example on the basis of chemically synthesized gold nanoparticles. In particular, the conjugated gold nanoparticles according to the present invention led to 18.95% to 47.15% GFP positive cells, wherein the comparative particles led only to 2.35% to 9.75% GFP positive cells.

With respect to the cell viability, it can be seen from FIG. 17B that the conjugated gold nanoparticles according to the present invention are linked with a sufficient viability when conjugation is performed with 9 μg transfection reagent and 1.5 μg or 3 μg nucleic acid molecules per well. The higher amount of polyethylenimine induced more apoptosis.

FIG. 18 shows the results of the studies in non-liver cell line HT1080 with 10 kDa linear PEI as the transfection reagent. As can be seen from FIG. 18A, the conjugated gold nanoparticles obtained by laser-ablation led to constant transfection rates with 32.65% to 39.6% GFP positive cells. With respect to the chemically synthesized gold nanoparticles, the percentage of GFP positive cells was significantly lower, namely in the range from 3.15% to 32.68%. Overall, the conjugated gold nanoparticles obtained by laser-ablation are linked with a higher transfection efficiency. Regarding the cell viability, it can be seen from FIG. 18B that the laser-ablated gold nanoparticles are linked with a significantly lower toxicity in comparison to the chemically synthesized gold nanoparticles.

FISH-Analysis of Episomal Persistence

Furthermore, studies on the basis of fluorescence in situ hybridization have been performed in HLE cells in order to investigate the episomal persistence of the nucleic acid molecules, in particular the vector, transferred on the basis of conjugated gold nanoparticles according to the present invention.

Transfection Procedure

300,000 cells (HLE) per well of a 6-well plate have been transfected with conjugated gold nanoparticles on the basis of 30 μg laser-ablated gold nanoparticles, 18 μg of branched PEI with a molecular mass of 25 kDa and 6 μg of nucleic acid molecules (pEPI-SM-S, cf. FIG. 3B).

Test Procedure

Subsequent to transfection and cultivation, FISH analysis has been performed. After ten weeks of cultivation with an initial neomycin selection for two weeks, the cells were arrested in metaphases with colcemid and FISH analysis was performed with a biotin-labeled probe for detection of the GFP cDNA.

Results

FIG. 19 shows an image obtained by fluorescence in situ hybridization (FISH). In this context, several GFP signals were detected (cf. small white dots as shown in FIG. 19). As cells arrested in metaphases were dropped onto slides, some of the DNA vectors that were episomally associated with the chromosomes detached from the chromosomes, so that either no or a single signal can be detected at the chromosomes. Evenly distributed signals on the chromosomes and/or chromatids are an indicator for the integration of the vector. Most of the chromosomes showed only one signal and only one chromosome showed integrated vectors. Therefore, the majority of the DNA comprising a S/MAR-element persisted episomally, despite continuous divisions of the fast growing HLE cells. The low risk of integration of the vector DNA into the genome is an indicator for a improved safety of the conjugated gold nanoparticles when used in gene therapy.

Factor Level Measurements in HLF/HT1080 Cells

Furthermore, studies have been performed in order to investigate the factor activity of coagulation factor FIX after transfection of HLF and HT1080 cells with conjugated laser-ablated gold nanoparticles.

Transfection Procedure

300,000 cells/well (HT1080, HLF) in a 6-well format have been transfected with conjugated gold nanoparticles on the basis of 30 μg laser-ablated gold nanoparticles, 18 μg transfection reagent (either Transporter5™ or 10 kDa linear PEI) and 6 μg nucleic acid molecules (either pEPI1-SM-S according to FIG. 3B as negative control, pEFi1-F9Pco according to FIG. 3C or pEFi1-F9co (map not shown, identical to pEFi1F9Pco with the exception that the nucleic acid sequence codes for factor FIX wt)).

Test Procedure

With respect to the cultivation of the cells, the cell culture medium was exchanged 4 and 24 hours after transfection and cells were kept in culture for three additional days. Cell culture supernatants were collected to determine the FIX activity.

Results

The respective results are depicted in FIG. 20. Both cell types transfected with the vector pEFi1-F9Pco were able to secrete factor FIX into the medium. In this context, a factor FIX activity between 12% and 304% was achieved. Higher factor activity was achieved in HT1080 cells. Even though the factor level in HLF cells was relatively low, with respect to a therapeutic approach it must be pointed out that already low percentages of factor activity are sufficient in order to compensate the negative effects and/or the phenotype of hemophilia. Against this background, also low factor activity as achieved in HLF cells could be sufficient with regard to a therapeutic effect in the treatment of hemophilia. Furthermore, it can be seen from the results that the use of a sequence coding for the padua variant of coagulation factor FIX is linked with a significantly higher production of factor FIX.

Factor Level Measurements in Primary Rat Hepatocytes

Furthermore, rat hepatocytes have been transfected with conjugated laser-ablated gold nanoparticles in order to investigate the transfection efficiency, on the one hand, and factor activity level, on the other hand. In this context, stable amounts of laser-ablated gold nanoparticles have been used. Furthermore, two different amounts (9 μg or 18 μg) of the transfection reagent (Transporter5™, Polysciences Europe GmbH, Hirschberg an der Bergstraße, DE or 25 kDa linear PEI) and nucleic acid molecules in amounts of 3 μg or 6 μg have been used in the conjugated gold nanoparticles.

Transfection Procedure

500,000 cells/well in a 6-well format have been transfected with conjugated gold nanoparticles on the basis of 30 μg laser-ablated gold nanoparticles, 9 μg or 18 μg transfection reagent (25 kDa linear PEI or Transporter5™) and 9 μg or 18 μg nucleic acid molecules (pEGFPc1, coding for eGFP under the control of a CMV promoter; pCDNA3F9Pco, coding for FIX padua under the control of the CMV promoter, cf. FIG. 3F; pEFi1EG, coding for eGFP under the control of an EF1-alpha promoter, in particular according to SEQ ID NO. 2; pEFi43EG, coding for eGFP under the control of an EF1-alpha promoter, in particular according to SEQ ID NO. 3; pEFi43F9Pco, coding sequence for FIX padua under the control of an EF1-alpha promoter, in particular according to SEQ ID NO. 13, cf. FIG. 3H). Furthermore, as a control transfection has been carried out without using gold nanoparticles as carrier systems.

Test Procedure

With respect to cultivation, cell culture medium was exchanged 4 and 24 hours after transfection and cells were incubated for additional three days. Subsequently, supernatants were collected for FIX activity analysis and GFP-transfected cells were analyzed for GFP expression by flow cytometry. On the basis of flow cytometry, the percentage of GFP positive cells, the mean fluorescence intensity (MFI) value and the percentage of non-apoptotic cells have been determined with respect to cells transfected with a GFP expressing vector.

Results

On the basis of FIG. 21A it can be seen that the conjugated laser-ablated gold nanoparticles have the ability to transfect mammalian liver cells, in particular primary rat hepatocytes. Furthermore, the mean fluorescence intensity (MFI) of the GFP in the cell has been determined (FIG. 21B). The determination of the mean fluorescence intensity also confirms that the conjugated gold nanoparticles obtained by laser ablation according to the present invention are suitable for the transfection of liver cells, in particular rat hepatocytes. Furthermore, on the basis of FIG. 21B it can be seen that on the basis of conjugated gold nanoparticles significantly higher amounts of nucleic acid molecules are present in the GFP positive cells. Furthermore, as can be seen from FIG. 21C, all approaches were linked with a sufficient cell viability.

FIG. 21D shows the results of the determination of the factor level of coagulation factor FIX. From the results obtained on the basis of the determination of the active factor level, it is evident that the use of conjugated laser-ablated gold nanoparticles leads to a significantly improved production of coagulation factor FIX in liver cells. In this context, with the vector pCDNA3F9Pco an active factor level of 48.5% was achieved, wherein transfection with the vector pEFi43F9Pco led to 13.4% active factor level, which is a still promising approach with respect to the realization of a therapeutic concept, in particular gene therapy, for the treatment of hemophilia.

Simultaneous Laser-Ablation and Conjugation

Furthermore, transfection studies have been performed in HLF cells and HT1080 cells in order to investigate the transfection efficiency of conjugated gold nanoparticles according to the present invention, wherein conjugation of the nanoparticles with polyethylenimine has been performed simultaneously with laser-ablation of the gold nanoparticles.

Preparation of PEI-conjugated gold nanoparticles

For this purpose, PEI-conjugated gold nanoparticles have been produced as described above according to the general experimental procedures. In this context, the buffer which has been used for pulsed-laser ablation in liquid contained different concentrations of branched polyethylenimine with a molecular mass of 25 kDa, namely concentrations of 10 μg/ml, 25 μg/ml, 50 μg/ml or 100 μg/ml. The gold nanoparticles as such had an average particle diameter of 5 nm, wherein conjugation during laser ablation increased the hydrodynamic diameter to a range of 14 to 22 nm, determined by dynamic light scattering.

Transfection Procedure

300,000 cells/well (HT1080, HLF) in a 6-well format have been transfected with conjugated gold nanoparticles on the basis of 30 μg laser-ablated and PEI-conjugated gold nanoparticles obtained as described above and 3 μg, 6 μg or 9 μg nucleic acid molecules (pEPI1-SM-S according to FIG. 3B).

Test Procedure

With respect to cultivation of the cells, 4 hours and 24 hours after transfection, the cell culture medium was exchanged and cells were kept in culture for additional three days. Thereafter, cells were collected and analyzed by flow cytometry in order to determine the percentage of GFP positive cells, the mean fluorescence intensities (MFI) and the percentage of non-apoptotic cells.

Results

The respective results with respect to the HLF cells are depicted in FIGS. 22A, 22B and 22C. FIG. 22A shows the percentage of GFP positive cells three days after transfection. A particularly efficient gene transfer was achieved with gold nanoparticles that were generated in solutions with 25 μg/ml, 50 μg/ml or 100 μg/ml of the polyethylenimine. Additionally, it can be seen that higher amounts of nucleic acid molecules are not necessarily linked with a higher percentage of GFP positive cells. Furthermore, the mean fluorescence index of the GFP positive cells was determined. In this context, transfection with gold nanoparticles generated in solutions with higher polyethylenimine concentrations led to higher mean fluorescence intensities in GFP positive cells, as depicted in FIG. 22B. Furthermore, as can be seen from FIG. 22C, all approaches led to a sufficient cell viability. Nevertheless, conjugated gold nanoparticles generated in higher concentrations of polyethylenimine were associated with slightly higher toxicity effects.

The results with respect to the HT1080 cells are depicted in FIGS. 23A, 23B and 23C. FIG. 23A relates to the percentage of GFP positive cells three days after transfection. The best results, i.e. the most efficient gene transfer, were achieved with conjugated gold nanoparticles generated in solutions containing 50 μg/ml or 100 μg/ml polyethylenimine. Furthermore, FIG. 23B shows that transfection with gold nanoparticles generated in solutions with higher polyethylenimine concentrations led to higher MFI values in GFP positive cells. Nevertheless, cells transfected with gold nanoparticles generated in the highest polyethylenimine concentration (100 μg/ml ) showed a decreasing MFI level. Against this background, overall, a favorable concentration of polyethylenimine in the solution for laser ablation in liquid seems to be 50 μg/ml. FIG. 23C contains the results with respect to the analysis of the cell viability on the basis of the determination of the percentage of non-apoptotic cells three days after transfection. It can be seen that all approaches led to a sufficient cell viability. Nevertheless, an increasing concentration of the polyethylenimine in the liquid for laser ablation is linked with a slightly increased cell toxicity.

Transfection Efficiency of Conjugated Gold Nanoparticles Comprising a Layer-By-Layer Assembly

Transfection studies have been performed in HLF and HT1080 cells in order to determine the transfection efficiency of conjugated laser-ablated gold nanoparticles comprising a layer-by-layer assembly with respect to the transfection reagents. In particular, it was analyzed whether a second layer of transfection reagent leads to higher transfection and/or gene transfer efficiencies.

Preparation of PEI-Conjugated Gold Nanoparticles

Laser-ablated gold nanoparticles with an average particle diameter of 5 nm have been prepared according to the general protocol for laser ablation. In a first step, the laser-ablated gold nanoparticles have been conjugated and/or coated with a first (inner) polyethylenimine layer comprising Transporter5™ (Polysciences Europe GmbH, Hirschberg an der Bergstraße, DE). After adding nucleic acid molecules on the basis of the vector pEPI-SM-S (cf. FIG. 3B), a second transfection reagent (either galactose-conjugated jetPEI®-hepatocyte or Transporter5™) as second (outer) layer has been added. With respect to the preparation of conjugated gold nanoparticles with a layer-by-layer assembly, reference is also made to FIG. 1B.

Transfection Procedure

300,000 cells/well (HT1080, HLF) in a 6-well format have been transfected with the above described conjugated gold nanoparticles on the basis of 30 μg laser-ablated gold nanoparticles, 9 μg Transporter5™ as first (inner) transfection reagent, 3 μg nucleic acid molecules (pEPI-SM-S, cf. FIG. 3B) and 0 μg, 0.1 μg, 0.3 μg, 1 μg, 3 μg or 9 μg of the second (outer) transfection reagent (either Transporter5™ or jetPEI®-hepatocyte).

Test Procedure

With respect to cultivation, the cell medium was exchanged 4 and 24 hours after transfection. Cells were kept in culture for two additional days and then analyzed by flow cytometry to determine the percentage of GFP expressing cells in order to determine the percentage of GFP positive cells, the mean fluorescence intensities (MFI) and the percentage of non-apoptotic cells.

Results

The results regarding HLF cells are depicted in FIGS. 24A, 24B and 24C. FIG. 24A shows the percentage of GFP positive cells three days after transfection. It can be seen that both variants of conjugated gold nanoparticles led to similar amounts of GFP positive cells. Nevertheless, slightly higher transfection rates with respect to amounts 1 μg, 3 μg or 9 μg with respect to the second transfection reagent were achieved with jetPEI®-hepatocyte (45%, 57%, 62% with jetPEI®-hepatocyte compared to 40%, 56% and 58% with Transporter5™). Overall, higher amounts of polyethylenimine led to larger amounts of GFP positive cells, independently from the polyethylenimine variant. The results with respect to the MFI values are depicted in FIG. 24B and confirm the results shown in FIG. 24A. Furthermore, it can be seen from FIG. 24C that higher amounts of polyethylenimine led to a higher toxicity and induced more apoptosis. Nevertheless, conjugated gold nanoparticles according to a preferred embodiment of the present invention on the basis of a layer-by-layer assembly are linked with sufficient cell viability.

FIG. 25 relates to the results obtained with HT1080 cells. FIG. 25A shows the percentage of GFP positive cells. It can be seen that both variants of polyethylenimine in the outer PEI layer led to similar results concerning the amount of GFP positive cells. The highest amount of GFP positive cells was achieved with the highest amount of polyethylenimine complexed with the gold nanoparticles. With respect to the MFI values, it can be seen from FIG. 25B that all approaches led to a sufficient amount of transferred DNA. Particularly high MFI levels were obtained with conjugated gold nanoparticles comprising Transporter5™ as outer layer in amounts of 3 μg or 9 μg. With respect to the cell viability, higher amounts of polyethylenimine were linked with a higher percentage of apoptotic cells (cf. FIG. 25C). Against this background, the use of smaller amounts of polyethylenimine in the outer layer seems to be more favorable.

Promoter Activity of SERPINA1 and hAAT Promoters

Transfection studies in HLF and HT1080 cells have been performed in order to investigate and compare the activity of the promoters SERPINA1 and hAAT in different target cell types. For this purpose, the target cells have been transfected with either the vector peAATEG according to FIG. 3M (hAAT promoter) or the vector peSEREG according to FIG. 3D (SERPINA1 promoter)

Transfection Procedure

300,000 cells/well (HT1080, HLF) in a 6-well format were transfected by admixing 3 μg or 6 μg nucleic acids and 18 μg transfection agent (Transporter5™, Polysciences Europe GmbH, Hirschberg an der Bergstraße, DE) to the cells.

Test procedure

With respect to cultivation, the cell medium was exchanged 4 and 24 hours after transfection. Cells were kept in culture for two additional days and then analyzed by flow cytometry to determine the percentage of GFP expressing cells, the mean fluorescence intensity value (MFI) and the percentage of non-apoptotic cells.

Results

FIGS. 26A, 26B and 26C relate to the studies in HLF cells. As can be seen from FIG. 26A, higher amounts of GFP positive cells were achieved with the vector peAATEG, despite the transfected DNA amount. Also with respect to the MFI value, the hAAT-promoter led to a higher expression level of the marker gene GFP in comparison to the SERPINA1-promoter (cf. FIG. 26B). As can be seen from FIG. 26C, sufficient cell viabilities were achieved on the basis of both approaches. Nevertheless, the combination of 18 μg Transporter5™ as the transfection reagent and an amount 3 μg DNA showed slightly higher toxicity effects with respect to both vectors.

FIGS. 27A, 27B and 27C relate to the studies in HT1080 cells. As can be seen from FIG. 27A, similar percentages of GFP positive cells have been achieved with the two test vectors. Nevertheless, slightly higher amounts of GFP positive cells were achieved with the vector carrying the hAAT-promoter. Furthermore, with respect to both vectors the lower amount of transfected vector DNA (3 μg/well) led to higher amounts of GFP positive cells. With respect to the mean fluorescence intensity values (MFIs) as depicted in FIG. 27B, higher values were observed in cells transfected with peSEREG. FIG. 27C shows the results of the analysis of the cell viability on the basis of the determination of the percentage of non-apoptotic cells three days after transfection. In this context, no relevant toxicity effects were observed with respect to both approaches.

TEM-Analyses of Laser-Ablated Gold Nanoparticles

In order to compare 5 nm chemically synthesized and laser-ablated gold nanoparticles with respect to their surface properties and size distribution, analyses on the basis of transmission electron microscopy (TEM) have been performed before and after conjugation.

For the purpose of TEM-analysis, unconjugated laser-ablated particles (FIG. 28A, bottom row) were diluted in phosphate buffer without any need of further stabilization. Chemically synthesized gold nanoparticles (FIG. 28A, upper row) have been stabilized in a solution comprising sodium citrate. Nevertheless, both methods lead to naked, unconjugated gold nanoparticles with an even particle size distribution (FIG. 28A).

Furthermore, TEM analyses have been performed after conjugating the particles with 25 kDa linear polyethylenimine as the ligand. For chemically synthesized gold nanoparticles, the transfection reagent was covalently bound to the surface of the nanoparticles by thiol groups. In contrast to this, the binding of the transfection reagent to the laser-ablated gold nanoparticles was based on electrostatic interactions. The TEM images (cf. FIG. 28B) revealed that the size distribution of the conjugated particles based on chemically synthesized gold nanoparticles varied in wide ranges (cf. FIG. 28B, upper row). In contrast to this, gold nanoparticles conjugated with polyethylenimine on the basis of laser-ablated gold nanoparticles comprise an even size distribution, which is particularly advantageous with respect to the use in gene therapy (cf. FIG. 28B, bottom row).
3. Conclusions

The current standard therapy for haemophilia comprises a life-long prophylactic administration of recombinant factors FVIII or FIX. However, frequent and expensive applications of the factors are necessary due to the short plasma half-life. On the basis of the present invention, a novel non-viral gene therapy approach for haemophilia B by transferring a normal copy of the mutated FVIII and/or FIX gene into the target cells, preferably hepatic cells, has been developed. This novel approach enables the target cells to produce the missing protein. Furthermore, this approach is applicable for any other monogenetic disorder associated with a lack of certain liver-specific or liver-expressed proteins due to a mutation coding for the gene of the respective protein.

According to the present invention, laser-ablated gold nanoparticles (AuNPs) as carriers for the vector DNA have been proven as superior over chemically produced gold nanoparticles with respect to the DNA transfer, compatibility and non-toxicity. Furthermore, the conjugated laser-ablated gold nanoparticles also are non-toxic, non-immunogenic and likely safer when compared to approaches with viral vectors.

With respect to the polyethylenimine, a particularly stable bond of the DNA to the gold nanoparticles as well as an efficient endosomal release of the DNA after cellular uptake has been achieved with linear PEI, preferably with a molecular weight of about 25 kDa.

Furthermore, a high-level production of clotting factors FVIII and/or FIX has been achieved by gene expression of the transgene under the control of different promoters, optimized for expression by in-/excluding introns, activating mutations and/or codon-optimization.

In order to further enhance specificity and efficiency of gene transfer, according to a particularly preferred embodiment of the present invention a layer-by-layer approach has been established, where two layers of PEI have been used. On this basis transfection efficiency is surprisingly increased. Furthermore, on the basis of such layer-by-layer approach or assembly the specificity of the conjugated gold nanoparticles with respect to the target cells can be improved. In particular, a layer-by-layer approach allows for cell specific targeting. In this context, an outer or second layer on the basis of a PEI variant that carries galactose residues (for example JetPEI®-Hepatocyte) to target the asialoglycoprotein receptor (ASGPR) has been proven suitable for an efficient targeting of the gold nanoparticles to hepatic cells or hepatocytes. Such second layer is not detrimental and can even increase gene transfer efficiency further.

Additionally, an improved method for the conjugation of laser-ablated gold nanoparticles has been found wherein conjugation is performed simultaneously with laser-ablation of the gold nanoparticles as such.

Finally, it was also found that vectors comprising the hAAT-promoter derived from human alpha-1 antitrypsin direct an efficient expression of coding sequences in different cell types, in particular liver cells and fibroblasts.

### LIST OF REFERENCE SIGNS

1 conjugated gold nanoparticles
1A conjugated gold nanoparticles with layer by layer assembly
2 gold nanoparticle
3 polyethylenimine
3A first polyethylenimine
3B second polyethylenimine
4 nucleic acid molecules
5 target cell (membrane)
6 endosome
7 importin
8 nuclear pore
9 nucleus

1. Conjugated gold nanoparticles, preferably for the use in the treatment, in particular a non-viral gene therapy, of a monogenetic disorder resulting from a mutation in a gene coding for a liver-specific and/or liver-expressed protein, comprising:
(a) laser-ablated gold nanoparticles;
(b) polyethylenimine (PEI) and/or derivatives and/or salts thereof; and
(c) at least one nucleic acid molecule, especially a vector, comprising (i) a promoter, preferably a promoter directing gene expression in mammalian, especially human cells and (ii) a coding sequence containing a nucleic acid sequence coding for a liver-specific and/or liver-expressed protein and/or preferably physiologically active domains and/or fragments thereof.

2. Conjugated gold nanoparticles according to claim 1, wherein the laser-ablated gold nanoparticles are obtained by pulse laser ablation in liquid (PLAL), especially wherein the pulsed laser irradiation has a wavelength in the range from 330 to 1,500 nm, preferably in the range from 800 to 1,200 nm.

3. Conjugated gold nanoparticles according claim 1 or 2, wherein the gold nanoparticles before conjugation have an average particle diameter dₚ [nm] in the range from 0.01 to 100 nm, in particular 0.05 to 80 nm, preferably 0.1 to 50 nm, particularly preferred 0.5 to 30 nm, even more preferred 1 to 15 nm, especially preferred 2 to 10 nm, preferably determined by analytical disc centrifugation (ADC) and/or transmission electron microscopy (TEM) and/or UV/VIS spectra.

4. Conjugated gold nanoparticles according to any of the preceding claims, wherein the gold nanoparticles before conjugation have a gold surface, wherein at least 90%, preferably at least 95% of said gold surface is freely accessible and not attached to any molecules.

5. Conjugated gold nanoparticles according to any of the preceding claims, wherein the conjugated gold nanoparticles have an average hydrodynamic diameter d_{hd} [nm] in the range from 0.05 to 150 nm, in particular 0.1 to 100 nm, preferably 0.5 to 80 nm, particularly preferred 1 to 50 nm, even more preferred 2 to 40 nm, especially preferred 10 to 30 nm, preferably determined by the method of dynamic light-scattering.

6. Conjugated gold nanoparticles according any of the preceding claims, wherein the polyethylenimine and/or derivatives and/or salts thereof are bound to the gold nanoparticles, preferably through electrostatic interaction with the surface of the gold nanoparticles.

7. Conjugated gold nanoparticles according to any of the preceding claims, wherein the polyethylenimine and/or derivatives and/or salts thereof are selected from the group of (i) linear polyethylenimines and/or derivatives and/or salts thereof; (ii) branched polyethylenimines and/or derivatives and/or salts thereof; and/or (iii) monosaccharide-conjugated, preferably galactose-conjugated polyethylenimines and/or derivatives and/or salts thereof.

8. Conjugated gold nanoparticles according to any of the preceding claims,
wherein the conjugated gold nanoparticles comprise at least two layers of polyethylenimine and/or derivatives and/or salts thereof; and/or
wherein the conjugated gold nanoparticles comprise alternating layers of polyethylenimine and/or derivatives and/or salts thereof and nucleic acid molecules, in particular an inner and an outer layer comprising polyethylenimine and/or derivatives and/or salts thereof with nucleic acid molecules assembled between the inner and the outer layer.

9. Conjugated gold nanoparticles according to claim 8, wherein the inner layer comprises linear and/or branched, preferably linear polyethylenimines and/or derivatives and/or salts thereof, and/or wherein the outer layer comprises linear, branched and/or monosaccharide-conjugated, preferably monosaccharide-conjugated polyethylenimines and/or derivatives and/or salts thereof.

10. Conjugated gold nanoparticles according to any of the preceding claims, wherein the polyethylenimine and/or derivatives and/or salts thereof have a number average molecular weight Mₙ in the range from 10 Da to 200 kDa, in particular from 100 kDa to 150 kDa, especially from 1 kDa to 100 kDa, particularly from 2 kDa to 50 kDa, preferably from 5 kDa to 40 kDa, more preferably from 8 kDa to 30 kDa, for example determined by means of gel permeation chromatography and/or according to DIN 55672-3:2016-03.

11. Conjugated gold nanoparticles according to any of the preceding claims, wherein the vector is a non-viral and/or a not integrating vector.

12. Conjugated gold nanoparticles according to any of the preceding claims, wherein the promoter is inducible and/or constitutive in mammalian cells, in particular human cells, preferably liver cells and/or fibroblasts, and/or wherein the promoter directs a tissue-specific, in particular liver-specific expression of the coding sequence.

13. Conjugated gold nanoparticles according to any of the preceding claims, wherein the promoter is derived from the gene coding for human Elongation Factor-1 alpha (EF1a) and/or wherein the promoter is derived from the human SERPINA1 promoter and/or wherein the promoter is derived from the hAAT (human alpha 1-antitrypsin) promoter and/or wherein the promoter is derived from Cytomegalovirus (CMV) and/or wherein the promoter is the CMV promoter.

14. Conjugated gold nanoparticles according to any of the preceding claims, wherein the promoter comprises a nucleotide sequence according to SEQ ID NO. 1, SEQ ID NO. 2, SEQ ID NO. 3, SEQ ID NO. 4 and/or SEQ ID NO. 5, preferably SEQ ID NO. 2, SEQ ID NO. 3 and/or SEQ ID NO. 4, and/or wherein the promoter comprises a nucleic acid sequence having at least 85%, in particular at least 90%, preferably at least 95% identity with SEQ ID NO. 1, SEQ ID NO. 2, SEQ ID NO. 3, SEQ ID NO. 4 and/or SEQ ID NO. 5, preferably SEQ ID NO. 2, SEQ ID NO. 3 and/or SEQ ID NO. 4.

15. Conjugated gold nanoparticles according to any of the preceding claims, wherein the vector contains at least one further cis-regulatory element, especially at least one further transcriptional enhancer.

16. Conjugated gold nanoparticles according to claim 15, wherein the cis-regulatory element is derived from the apolipoprotein E gene, in particular the apolipoprotein E hepatic locus control region and/or wherein the cis-regulatory element has a nucleotide sequence according to SEQ ID NO. 6 and/or wherein the cis-regulatory element has a nucleic acid sequence having at least 85%, in particular at least 90%, preferably at least 95% identity with SEQ ID NO. 6.

17. Conjugated gold nanoparticles according to any of the preceding claims, wherein the nucleic acid sequence of the coding sequence is codon-optimized for human gene expression and/or human codon usage.

18. Conjugated gold nanoparticles according to any of the preceding claims, wherein the coding sequence comprises a nucleic acid sequence coding for a liver-specific and/or liver-expressed protein selected from proteins produced and/or predominantly expressed in the liver.

19. Conjugated gold nanoparticles according to any of the preceding claims, wherein the coding sequence comprises a nucleic acid sequence coding for a liver-specific and/or liver-expressed protein selected from the group of:
(i) major plasma proteins, in particular human serum albumin, alpha-fetoprotein, soluble plasma fibronectin, C-reactive protein and/or preferably physiologically active domains and/or fragments thereof;
(ii) stimulators and/or factors for coagulation, preferably coagulation factor FVII, FVIII, FIX, FX, FXI, FXII, FXIII and/or preferably physiologically active domains and/or fragments thereof, preferably FVIII, FIX and/or preferably physiologically active domains and/or fragments thereof;
(iii) inhibitors of coagulation, preferably alpha2-macroglobulin, alpha1-antitrypsin, antithrombin III, protein S, protein C and/or preferably physiologically active domains and/or fragments thereof;
(iv) stimulators of fibrinolysis, preferably plasminogen and/or preferably physiologically active domains and/or fragments thereof; and/or
(v) inhibitors of fibrinolysis, preferably alpha2-antiplasmin and/or preferably physiologically active domains and/or fragments thereof; and/or
(vi) proteins of the amino acid metabolism, in particular fumarylacetoacetate hydrolase, p-hydroxyphenylpyruvate hydroxylase and/or phenylalanine-4-hydroxylase; and/or
(vii) antiproteases, in particular alpha-1 antitrypsin; and/or
(viii) proteins of the bilirubin metabolism, in particular uridine diphospho-glucuronosyltransferase; and/or
(ix) proteins of the urea cycle, in particular arginase, argininosuccinate synthase and/or ornithine transcarbamylase; and/or
(x) proteins of the carbohydrate metabolism, in particular alpha-glucan phosphorylase, amylo-1,6-glucosidase and/or glucose-6-phosphatase; and/or
(xi) proteins of the proteoglycan metabolism, in particular idursulfase; and/or
(xii) proteins of the sphingolipid metabolism, in particular glucocerebrosidase; and/or
(xiii) proteins involved in transport processes, in particular p-type ATPase, cystic fibrosis transmembrane regulator and/or low-density lipoprotein (LDL) receptor; and/or
(xiv) proteins involved in lipometabolism and/or proteins linked with monogenetic lipometabolic disorders.

20. Conjugated gold nanoparticles according to any of the preceding claims, wherein the coding sequence comprises a nucleic acid sequence coding for a coagulation factor, in particular coagulation factor FVII, FVIII, FIX, FX, FXI, FXII, FXIII and/or preferably physiologically active domains and/or fragments thereof, preferably coagulation factor FVIII, FIX and/or preferably physiologically active domains and/or fragments thereof.

21. Conjugated gold nanoparticles according to any of the preceding claims,
wherein the coding sequence has a nucleotide sequence coding for coagulation factor FVIII and/or preferably physiologically active domains and/or fragments thereof and/or
wherein the coding sequence has a nucleotide sequence according to SEQ ID NO. 7 an/or SEQ ID NO. 8, preferably SEQ ID NO. 8, and/or wherein the coding sequence has a nucleotide sequence having at least 85%, in particular at least 90%, preferably at least 95% identity with SEQ ID NO. 7 and/or SEQ ID NO. 8, preferably SEQ ID NO. 8, and/or
wherein the coding sequence has a nucleic acid sequence corresponding to the nucleic acid sequence of the native cDNA coding for human coagulation factor FVIII and/or wherein the coding sequence codes for a protein having an amino acid sequence according to SEQ ID NO. 9 and/or an amino acid sequence having at least 85%, in particular at least 90%, preferably at least 95% identity with SEQ ID NO. 9.

22. Conjugated gold nanoparticles according to any of the preceding claims,
wherein the coding sequence comprises a nucleic acid sequence coding for coagulation factor FIX and/or preferably physiologically active domains and/or fragments thereof; and/or
wherein the coding sequence has a nucleotide acid sequence according to SEQ ID NO. 10, SEQ ID NO. 11 and/or SEQ ID NO. 12 and/or a nucleotide sequence having at least 85%, in particular at least 90%, preferably at least 95% identity with SEQ ID NO. 10, SEQ ID NO. 11 and/or SEQ ID NO. 12; and/or
wherein the coding sequence has a nucleotide sequence corresponding to the nucleotide sequence of the native cDNA coding for human coagulation factor FIX and/or wherein the coding sequence codes for a protein having an amino acid sequence according to SEQ ID NO. 13 and/or SEQ ID NO. 14 and/or an amino acid sequence having at least 85%, in particular at least 90%, preferably at least 95% identity with SEQ ID NO. 13 and/or SEQ ID NO. 14.

23. Conjugated gold nanoparticles according to any of the preceding claims, wherein the coding sequence has a nucleotide sequence coding for a fusion protein on the basis of a coagulation factor and/or preferably physiologically active domains and/or fragments thereof, in particular coagulation factor FVIII and/or FIX, preferably coagulation factor FIX, and an albumin and/or domains and/or fragments thereof.

24. Conjugated gold nanoparticles according to any of the preceding claims,
wherein the coding sequence has a nucleotide sequence according to SEQ ID NO. 15 and/or SEQ ID NO. 16 and/or a nucleotide sequence having at least 85%, in particular at least 90%, preferably at least 95% identity with SEQ ID NO. 15 and/or SEQ ID NO. 16 and/or
wherein the coding sequence codes for a protein having an amino acid sequence according to SEQ ID NO. 17 and/or SEQ ID NO. 18 and/or an amino acid sequence having at least 85%, in particular at least 90%, preferably at least 95% identity with SEQ ID NO. 17 and/or SEQ ID NO. 18.

25. Conjugated gold nanoparticles according to any of the preceding claims, wherein the vector comprises a scaffold/matrix attachment region, in particular a scaffold/matrix attachment region derived from the gene coding for human Interferon-beta (IFN-beta).

26. Conjugated gold nanoparticles according to claim 25, wherein the scaffold/matrix attachment region has a nucleotide sequence according to SEQ ID NO. 19 and/or SEQ ID NO. 20, in particular SEQ ID NO. 20, and/or a nucleotide sequence having at least 85%, in particular at least 90%, preferably at least 95% identity with SEQ ID NO. 19 and/or SEQ ID NO. 20, in particular SEQ ID NO. 20.

27. Conjugated gold nanoparticles according to any of the preceding claims, wherein the weight related ratio of polyethylenimine to nucleic acid molecules is in the range of from 1:100 to 60:1, in particular from 1:50 to 40:1, especially from 1:30 to 20:1, preferably from 1:10 to 10:1, more preferred from 1:1 to 10:1, further preferred from 1:1 to 6:1.

28. Conjugated gold nanoparticles according to any of the preceding claims, wherein the weight related ratio of polyethylenimine and/or derivatives and/or salts thereof to gold nanoparticles is in the range of from 1:100 to 100: 1, especially from 1:50 to 50:1, preferably from 1:30 to 20:1, in particular preferred from 1:20 to 10:1, even more preferred from 1:10 to 1:1.

29. Conjugated gold nanoparticles according to any of the preceding claims for the use in the treatment, in particular a non-viral gene therapy, of a monogenetic disorder resulting from a mutation in a gene coding for a liver-specific and/or liver-expressed protein.

30. Conjugated gold nanoparticles according to claim 29, wherein the monogenetic disorder is associated with an impaired and/or reduced hemostasis and/or blood clotting, especially wherein the disorder is a hemophilia, in particular hemophilia A and/or hemophilia B.

31. Use of conjugated gold nanoparticles according to any of the preceding claims
in the treatment, in particular a non-viral gene therapy, of a monogenetic disorder resulting from a mutation in a gene coding for a liver-specific and/or liver-expressed protein, and/or
for the preparation of a medicament for the treatment, in particular a non-viral gene therapy, of a monogenetic disorder resulting from a mutation in a gene coding for a liver-specific and/or liver-expressed protein, preferably via transfection.

32. Use according to claim 31, wherein the monogenetic disorder is associated with an impaired and/or reduced hemostasis and/or blood clotting, especially wherein the disorder is a hemophilia, in particular hemophilia A and/or hemophilia B.

33. Method for the preparation of conjugated gold nanoparticles, wherein the gold nanoparticles comprise polyethylenimine (PEI) and/or derivatives and/or salts thereof, in particular conjugated gold nanoparticles according to any of claims 1 to 30, and
wherein the method comprises the following method steps:
(a) providing unconjugated (naked) gold nanoparticles by laser ablation, especially pulsed laser ablation in liquid (PLAL);
(b) conjugating the gold nanoparticles with polyethylenimine (PEI) and/or derivatives and/or salts thereof; and
(c) conjugating the gold nanoparticles with nucleic acid molecules, especially a vector, comprising (i) a promoter, preferably a promoter directing gene expression in mammalian, especially human cells, and (ii) a coding sequence containing a nucleic acid sequence coding for a liver-specific and/or liver-expressed protein and/or preferably physiologically active domains and/or fragments thereof, wherein mutations in the nucleic acid sequence coding for the liver-specific and/or liver-expressed protein are associated with a monogenetic disorder, preferably by admixing the gold nanoparticles with the nucleic acid molecules.

34. Method according to claim 33,
wherein the pulsed laser irradiation has a wavelength in the range from 330 to 1,500 nm, preferably in the range from 800 to 1,200 nm; and/or
wherein the pulse energy is in the range of 1 to 1,000 μJ, especially 5 to 500 μJ, particularly 10 to 250 μJ, preferably 50 to 200 μJ, even more preferred 90 to 150 μJ; and/or
wherein the pulse repetition rate is in the range of 1 to 1,000 kHz, especially 5 to 500 kHz, particularly 10 to 250 kHz, preferably 50 to 200 kHz, even more preferred 80 to 150 kHz; and/or
wherein the pulse duration is in the range of 0.1 to 500 ps, especially 0.5 to 100 ps, particularly 1 to 50 ps, preferably 2 to 25 ps, even more preferred 5 to 15 ps.

35. Method according to claim 33 or 34, wherein the gold nanoparticles are adjusted to an average particle diameter dₚ [nm] in the range from 0.01 to 100 nm, in particular 0.05 to 80 nm, preferably 0.1 to 60 nm, particularly preferred 0.5 to 50 nm, even more preferred 1 to 25 nm, especially preferred 2 to 10 nm, preferably determined by analytical disc centrifugation (ADC) and/or transmission electron microscopy (TEM) and/or UV/VIS spectra.

36. Method according to any of claims 33 to 35, wherein laser ablation is performed with a gold target, especially wherein the gold target has a thickness in the range of 0.1 to 20,000 μm, especially 1 to 15,000 μm, particularly 10 to 10,000 μm, preferably 50 to 8,000 μm, even more preferred 100 to 5,000 μm.

37. Method according to any of claims 33 to 36, wherein laser ablation, in particular pulsed laser ablation in liquid, is performed in (i) purified water and/or (ii) phosphate based buffer, preferably sodium phosphate buffer (NaPB) and/or phosphate buffer saline (PBS) as liquid.

38. Method according to any of claims 33 to 37, wherein conjugating the gold nanoparticles with polyethylenimine and/or derivatives and/or salts thereof is performed simultaneously with method step (a) and/or laser ablation of the unconjugated (naked) gold nanoparticles, wherein the laser ablation, in particular the pulsed laser ablation in liquid, is performed in the presence of polyethylenimine and/or derivatives and/or salts thereof.

39. Method according to claim 38, wherein polyethylenimine and/or derivatives and/or salts thereof is added to the liquid, especially wherein polyethylenimine and/or derivatives and/or salts thereof is added to a concentration in the range from 0.1 to 1.000 μg/ml, especially in the range from 0.5 to 800 μg/ml, preferably in the range from 5 to 500 μg/ml, in particular in the range from 10 to 300 μg/ml, particularly preferred in the range from 20 to 200 μg/ml, based on the liquid for pulsed laser ablation.

40. Method according to any of claims 33 to 37, wherein conjugating the gold nanoparticles with polyethylenimine and/or derivatives and/or salts thereof is performed by admixing the laser-ablated gold nanoparticles with polyethylenimine and/or derivatives and/or salts thereof, especially wherein admixing the gold nanoparticles with polyethylenimine and/or derivatives and/or salts thereof is performed as a separate method step and/or simultaneously with method step (c).

41. Method according to any of claims 33 to 40, wherein polyethylenimine and/or derivatives and/or salts thereof and gold nanoparticles are employed in a weight related ratio in the range from 1:100 to 100:1, especially from 1:50 to 50:1, preferably from 1:30 to 20:1, in particular preferred from 1:20 to 10:1, even more preferred from 1:10 to 1:1.

42. Method according to any of claims 33 to 41, wherein polyethylenimine and/or derivatives and/or salts thereof and nucleic acid molecules are employed in a weight related ratio of polyethylenimine and/or derivatives and/or salts thereof to nucleic acid molecules in the range from 1:100 to 150:1, especially from 1:50 to 100:1, preferably from 1:20 to 50:1, in particular preferred from, 1:10 to 20:1, even more preferred from 1:1 to 10:1.

43. Method according to any of claims 33 to 42, wherein subsequent to method steps (a) to (c) a method step further method step (d) is performed, wherein in method step (d) the particles obtained by method steps (a) to (c) are conjugated with a further outer layer comprising polyethylenimine and/or derivatives and/or salts thereof, preferably galactose-conjugated polyethylenimine and/or derivatives and/or salts thereof.

44. Nanoparticle-based delivery system for a coding sequence, preferably for the use in the treatment, in particular non-viral gene therapy, of a monogenetic disorder resulting from a mutation in a gene coding for a liver-specific and/or liver-expressed protein, wherein the delivery system comprises a multitude of conjugated gold nanoparticles according to the preceding claims and a physiologically and/or pharmaceutically acceptable carrier.

45. Nanoparticle-based delivery system, wherein the nanoparticle-based delivery system is prepared for a systemic application, in particular an intravenous and/or oral, preferably systemic application.

46. Nanoparticle-based delivery system according to claim 44 or 45, wherein the disorder is associated with an impaired and/or reduced hemostasis and/or blood clotting, especially wherein the disorder is a hemophilia, in particular hemophilia A and/or hemophilia B.

47. Use of a delivery system according to any of claims 44 to 46 in the treatment, in particular a non-viral gene therapy, of a monogenetic disorder resulting from a mutation in a gene coding for a liver-specific and/or liver-expressed protein and/or for the preparation of a medicament for the treatment of a monogenetic disorder resulting from a mutation in a gene coding for a liver-specific and/or liver-expressed protein.

48. Use according to claim 47, wherein the monogenetic disorder is associated with an impaired and/or reduced hemostasis and/or blood clotting, especially wherein the disorder is a hemophilia, in particular hemophilia A and/or hemophilia B.

49. Method for the transfection of target cells, especially mammalian cells, preferably human cells, preferably liver-cells and/or fibroblasts, wherein conjugated gold nanoparticles according to any of claims 1 to 30 are used in that method.

50. Transfected cell, preferably mammalian, in particular human cell, especially for the use in the treatment, in particular non-viral gene therapy, of a monogenetic disorder resulting from a mutation in a gene coding for a liver-specific and/or liver-expressed protein, wherein transfection has been performed with conjugated gold nanoparticles according to any of claims 1 to 30 and/or wherein the transfected cell comprises conjugated gold nanoparticles according to any of claims 1 to 30.

51. Vector, in particular non-viral vector, preferably for the expression of a liver-specific and/or liver-expressed protein and/or preferably physiologically active domains and/or fragments thereof in a patient suffering from a monogenetic disorder caused by a mutation in the gene coding for the liver-specific and/or liver-expressed protein, wherein the vector comprises:
(a) a promoter, wherein the promoter is derived from a human gene;
(b) a coding sequence containing a nucleic acid sequence coding for a liver-specific and/or liver-expressed protein and/or preferably physiologically active domains and/or fragments thereof, wherein mutations in the nucleic acid sequence coding for the liver-specific and/or liver-expressed protein are associated with a monogenetic disorder;
(c) a nucleic acid sequence derived from the scaffold/matrix attachment region of a eukaryotic, preferably human gene; and
(d) a transcriptional termination signal.

52. Vector according to claim 51 or 52, wherein the promoter is derived from the gene coding to human Elongation Factor-1 alpha (EF1a) and/or wherein the promoter is derived from the human SERPINA1 promoter and/or wherein the promoter is derived from the hAAT (human 1-antitrypsin) promoter.

53. Vector according to claim 51, wherein the promoter comprises a nucleotide sequence according to SEQ ID NO. 3, SEQ ID NO. 4 and/or SEQ ID NO. 5, especially SEQ ID NO. 3 and/or SEQ ID NO. 4, and/or wherein the promoter comprises a nucleic acid sequence having at least 85%, in particular at least 90%, preferably at least 95% identity with SEQ ID NO. 3, SEQ ID NO. 4 and/or SEQ ID NO. 5, especially SEQ ID NO. 3 and/or SEQ ID NO. 4.

54. Vector according to any of claims 51 to 53, wherein the vector contains at least one further cis-regulatory element, especially at least one further transcriptional enhancer.

55. Vector according to any of claims 51 to 54, wherein the cis-regulatory element is derived from the apolipoprotein E gene, in particular the apolipoprotein E hepatic locus control region and/or wherein the cis-regulatory element has a nucleotide sequence according to SEQ ID NO. 6 and/or wherein the promoter has a nucleic acid sequence having at least 85%, in particular at least 90%, preferably at least 95% identity with SEQ ID NO. 6.


SEQUENCE LISTING (22 sequences)

SEQ ID NO: 1 - DNA, 588 bp, Cytomegalovirus
  CMV Promoter (1)..(588): CMV Promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagat

SEQ ID NO: 2 - DNA, 458 bp, Homo sapiens
  EF1alpha_promoter_opt_splice_sites (1)..(458): promoter derived from human EF1alpha plus splice sites derived from human EF1alpha
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatcgcccttgcgtgccttgaattacttccacgcccctggctgcagtacgtgattcttgatcccgattctccttggaatttgccctttttgagtttggatcttggttcattctcaagcctcagacagtggttcaaagtttttttcttccatttcaggtgtcgaggcctgaattcgtcgactg

SEQ ID NO: 3 - DNA, 226 bp, Homo sapiens
  EFi43_promoter (1)..(226): core promoter EFi43 derived from human EF1-alpha
ctcgagcgatcgatggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgg

SEQ ID NO: 4 - DNA, 397 bp, Homo sapiens
  hAAT (1)..(397): promoter dervived from human alpha-1 antitrypsin
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 5 - DNA, 428 bp, Homo sapiens
  SERPINA1 (1)..(428): promoter derived from human SERPINA1
ccaggtcctatgacacacacctccccagtgcacacagacctgcccaactgtggggctgcccactgggcatttcataggtggctcagtcctcttccctctgcagctggccccagaaacctgccagttattggtgccaggtctgtgccaggagggcgaggcctgtcatttctagtaatcctctgggcagtgtgactgtacctcttgcggcaactcaaagggagagggtgacttgtcccgggtcacagagctgaaagggcaggtacaacaggtgacatgccgggctgtctgagtttatgagggcccagtcttgtgtctgccgggcaatgagcaaggctccttcctgtccaagctccccgcccctccccagcctactgcctccacccgaagtctacttcctgggtgggcaggaactgggcactgtgcccagg

SEQ ID NO: 6 - DNA, 154 bp, Homo sapiens
  ApoEHCR (1)..(154): hepatic control region derived from Apolipoprotein E
gtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggc

SEQ ID NO: 7 - DNA, 4374 bp, Homo sapiens
  FVIII_wt (1)..(4374): factor FVIII wt SQ with deleted B-domain
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaatccaccagtcttaaaaagacaccagagagaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 8 - DNA, 4374 bp, Homo sapiens
  FVIII (1)..(4374): coagulation factor FVIII SQ with deleted B-domain, codon optimized
atgcagatcgagctctctacctgcttcttcctgtgcctgctgcggttctgcttcagcgccaccagacggtactatctgggcgccgtggaactgagctgggactacatgcagagcgacctgggcgagctgcccgtggatgccagattccctccaagagtgcccaagagcttccccttcaacacctccgtggtgtataagaaaaccctgttcgtggagttcaccgaccacctgttcaatatcgccaagcccagacccccctggatgggcctgctgggacctacaattcaggccgaggtgtacgacaccgtcgtgatcaccctgaagaacatggccagccaccccgtgtctctgcatgccgtgggagtgtcctactggaaggcctctgagggcgccgagtacgacgatcagaccagccagcgcgagaaagaggacgacaaggtgttccctggcggcagccacacctacgtgtggcaggtgctgaaagaaaacggccccatggcctccgaccctctgtgcctgacatacagctacctgagccacgtggacctcgtgaaggacctgaacagcggcctgatcggagccctgctcgtgtgtagagagggcagcctggccaaagagaaaacccagaccctgcacaagttcatcctgctgttcgccgtgttcgacgagggcaagagctggcacagcgagacaaagaacagcctgatgcaggaccgggacgccgcctctgctagagcctggcccaaaatgcacaccgtgaacggctacgtgaacagaagcctgcccggactgatcggctgccaccggaagtctgtgtactggcacgtgatcggcatgggcaccacccctgaggtgcacagcatctttctggaaggacacacctttctcgtgcggaaccaccggcaggccagcctggaaatcagccctatcaccttcctgaccgcccagacactgctgatggacctgggccagtttctgctgttctgccacatcagctcccaccagcacgacggcatggaagcctacgtgaaggtggacagctgccccgaggaaccccagctgcggatgaagaacaacgaggaagccgaggactacgacgacgacctgaccgacagcgagatggacgtggtgcgcttcgacgacgataacagccccagcttcatccagatcagaagcgtggccaagaagcaccccaagacctgggtgcactatatcgccgccgaggaagaggactgggattacgcccctctggtgctggcccccgacgacagaagctacaagagccagtacctgaacaatggcccccagcggatcggccggaagtacaagaaagtgcggttcatggcctacaccgacgagacattcaagaccagagaggccatccagcacgagagcggcatcctgggccctctgctgtatggcgaagtgggcgacaccctgctgatcatcttcaagaaccaggccagcagaccctacaacatctaccctcacggcatcaccgacgtgcggcccctgtattctcggagactgcccaagggcgtgaaacacctgaaggacttccccatcctgcccggcgagatcttcaagtacaagtggaccgtgaccgtggaagatggccccaccaagagcgaccccagatgcctgacacggtactacagcagcttcgtgaacatggaacgggacctggcctccggcctgattggcccactgctgatctgctacaaagaaagcgtggaccagcggggcaaccagatcatgagcgacaagcggaacgtgatcctgtttagcgtgttcgatgagaaccggtcctggtatctgaccgagaatatccagcggttcctgcccaaccctgccggcgtgcagctggaagatcctgagttccaggcctccaacatcatgcactccatcaatggctatgtgttcgacagcctgcagctgagcgtgtgcctgcacgaggtggcctactggtacatcctgagcatcggggcccagaccgacttcctgtccgtgttcttctccggctacaccttcaagcacaagatggtgtacgaggataccctgaccctgttcccctttagcggcgaaaccgtgttcatgagcatggaaaaccccggcctgtggatcctgggctgccacaacagcgacttccggaacagaggcatgaccgccctgctgaaggtgtccagctgcgacaagaacaccggcgactactacgaggacagctatgaggacatcagcgcctacctgctgagcaagaacaatgccatcgagcccagaagcttcagccagaacccccccgtgctgaagcggcaccagagagagatcacccggaccaccctgcagtccgaccaggaagagatcgattacgacgacaccatcagcgtggaaatgaagaaagaagatttcgacatctacgacgaggacgagaaccagagcccccggtcctttcagaaaaagacccggcactacttcattgccgctgtggaacggctgtgggactacggcatgagcagcagccctcacgtgctgagaaacagggcccagagcggcagcgtgccccagttcaaaaaggtggtgttccaggagtttaccgacggcagcttcacccagcctctgtaccggggagagctgaacgagcacctgggactgctgggcccctatatcagagccgaagtggaagataacatcatggtcaccttccggaatcaggcctcccggccctacagcttctacagctccctgatcagctacgaagaggaccagagacagggcgctgagccccggaagaacttcgtgaagcccaacgagactaagacctacttttggaaggtgcagcaccacatggcccctacaaaggacgagttcgactgcaaggcctgggcctacttctccgatgtggacctggaaaaggacgtgcactctgggctgatcggccccctgctcgtgtgccacaccaacaccctgaatcccgcccacggcagacaagtgacagtgcaggaatttgccctgttcttcaccatcttcgacgaaacaaagagctggtacttcaccgaaaacatggaaagaaactgccgggctccctgcaacatccagatggaagatcccaccttcaaagagaactaccggttccacgccatcaacggctacatcatggacacactgcccggcctcgtgatggctcaggatcagcggatccggtggtatctgctgtccatgggctccaacgagaacatccacagcatccacttcagcggccacgtgttcaccgtgcggaaaaaagaagagtacaaaatggccctgtataacctgtaccccggcgtgttcgagacagtggaaatgctgcctagcaaggccggcatctggcgggtggaatgtctgatcggcgagcatctgcacgctgggatgagcacactgtttctggtgtactccaacaagtgccagacacctctgggcatggcctctggccacatccgggactttcagatcacagccagcggccagtatggccagtgggccccaaaactggccagactgcactacagcggcagcatcaacgcctggtccaccaaagagcccttcagctggatcaaggtggacctgctggctcccatgatcatccacggaatcaagacccagggcgccagacagaagttcagcagcctgtatatcagccagttcatcatcatgtactccctggacggcaagaagtggcagacctaccggggcaatagcaccggcaccctgatggtgttcttcggcaacgtggactccagcggcattaagcacaacatcttcaacccccccatcattgcccggtacatccggctgcaccccacccactacagcatccggtccaccctgagaatggaactgatgggctgcgacctgaactcctgcagcatgcccctggggatggaaagcaaggccatctccgacgcccagatcaccgcctccagctacttcaccaacatgttcgccacctggtcccccagcaaggcccggctgcatctgcagggcagaagcaatgcttggaggccccaagtgaacaaccccaaagaatggctgcaggtggacttccagaaaaccatgaaagtgaccggcgtgaccacccagggcgtgaagtctctgctgacctctatgtacgtgaaagagttcctgatctccagcagccaggacggccaccagtggaccctgtttttccagaacggcaaagtgaaagtgtttcaggggaaccaggactccttcacccccgtcgtgaatagcctggaccctccactgctgaccagataccttcgaatccaccctcagtcttgggtgcaccagattgctctgcggatggaagtgctgggatgcgaggcccaggacctgtactga

SEQ ID NO: 9 - protein, 1457 aa, Homo sapiens
  FVIII (1)..(1457): Factor FVIII SQ with deleted B-domain
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 10 - DNA, 1386 bp, Homo sapiens
  FIX_wt (1)..(1386): coagulation factor FIX SQ wt
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 11 - DNA, 1386 bp, Homo sapiens
  FIX (1)..(1386): Factor FIX, codon optimized
atgcaacgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaatggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgccgtgcctttcccatgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgccaagcctggacagttcccgtggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgagaagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagctgacatga

SEQ ID NO: 12 - DNA, 1386 bp, Homo sapiens
  FIX (1)..(1386): Factor FIX padua, codon optimized
atgcaacgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaatggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgccgtgcctttcccatgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgccaagcctggacagttcccgtggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgctgagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagcttacatga

SEQ ID NO: 13 - protein, 461 aa, Homo sapiens
  FIX (1)..(461): amino acid sequence factor FIX
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 14 - protein, 460 aa, Homo sapiens
  FIX_padua (1)..(460): amino acid sequence factor FIX padua
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKL

SEQ ID NO: 15 - DNA, 3195 bp, Homo sapiens
  FIX_albumin (1)..(3195): fusion of FIX and albumin, codon optimized
atgcaacgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaatggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgccgtgcctttcccatgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgccaagcctggacagttcccgtggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgagaagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagcttacacccgtgtctcagacctctaagctgacccgggctgaaactgtgtttccagatgtggacgcccacaagagcgaggtggcccacagattcaaggacctgggagaagagaacttcaaggctctggtgctgatcgccttcgctcagtacctccagcagtgcccattcgaggaccatgtgaagctggtcaacgaagtgaccgagttcgccaagacctgcgtggccgatgagtccgccgagaactgtgataagagcctgcacaccctgttcggcgacaagctgtgtacagtggccacactgagagaaacctacggcgagatggccgactgctgcgccaaacaagagcccgagagaaacgagtgcttcctccagcacaaggacgataaccccaacctgcctagactcgtgcggcccgaagtggatgtcatgtgcaccgccttccacgacaacgaggaaaccttcctgaagaagtacctgtacgagatcgccagacggcacccctacttttacgcccctgagctgctgttctttgccaagagatacaaggccgccttcaccgagtgttgccaggccgctgataaggccgcttgtctgctgcctaagctggatgagctgcgcgacgagggcaaagcctcttctgccaagcagagactgaagtgcgccagcctccagaagtttggcgagagagcctttaaggcctgggccgtcgctagactgagccagagatttcccaaggccgagtttgccgaggtgtccaagctggttaccgacctgaccaaggtgcacacagagtgctgtcacggcgatctgctggaatgcgccgacgatagagccgatctggccaagtacatctgcgagaatcaggacagcatcagctccaagctgaaagaatgctgcgagaagcccctgctcgaaaagagccactgtatcgctgaggtggaaaacgacgagatgcccgccgatctgccttctctggccgccgattttgtggaaagcaaggacgtgtgcaagaattacgccgaggccaaggatgtgttcctgggcatgtttctgtatgagtacgcccgcagacaccccgactactctgttgtgctgctgctgagactggccaaaacctacgagactaccctggaaaagtgctgtgccgccgctgatcctcacgagtgttacgccaaagtgttcgacgagttcaagccactggtggaagaaccccagaacctgatcaaacagaactgcgaactgttcgagcagctgggcgagtacaagttccagaacgccctgctcgtgcggtacaccaagaaggtgccccaggtttcaacccctacactggttgaggtgtcccggaacctgggcaaagtgggcagcaagtgttgcaagcaccctgaggccaagagaatgccctgcgccgaggattacctgagcgtcgtgctgaatcagctgtgcgtgctgcacgagaaaacccctgtgtccgacagagtgaccaagtgctgtaccgagagcctggtcaacagacggccttgctttagcgcccttgaggtggacgagacatacgtgcccaaagagttcaacgccgagacattcaccttccacgccgacatctgtaccctgagcgagaaagagcggcagatcaagaaacagaccgctctggtggaactggtcaagcacaagcccaaggccaccaaagaacaactgaaggccgtgatggacgacttcgccgcctttgtcgagaagtgctgcaaggccgatgacaaagagacatgcttcgccgaagagggaaagaaactggtggccgcctctcaagccgctctgggactttaa

SEQ ID NO: 16 - DNA, 3195 bp, Homo sapiens
  FIX_albumin (1)..(3195): fusion of FIX padua and albumin, codon optimized
atgcaacgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaatggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgccgtgcctttcccatgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgccaagcctggacagttcccgtggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgctgagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagcttacacccgtgtctcagacctctaagctgacccgggctgaaactgtgtttccagatgtggacgcccacaagagcgaggtggcccacagattcaaggacctgggagaagagaacttcaaggctctggtgctgatcgccttcgctcagtacctccagcagtgcccattcgaggaccatgtgaagctggtcaacgaagtgaccgagttcgccaagacctgcgtggccgatgagtccgccgagaactgtgataagagcctgcacaccctgttcggcgacaagctgtgtacagtggccacactgagagaaacctacggcgagatggccgactgctgcgccaaacaagagcccgagagaaacgagtgcttcctccagcacaaggacgataaccccaacctgcctagactcgtgcggcccgaagtggatgtcatgtgcaccgccttccacgacaacgaggaaaccttcctgaagaagtacctgtacgagatcgccagacggcacccctacttttacgcccctgagctgctgttctttgccaagagatacaaggccgccttcaccgagtgttgccaggccgctgataaggccgcttgtctgctgcctaagctggatgagctgcgcgacgagggcaaagcctcttctgccaagcagagactgaagtgcgccagcctccagaagtttggcgagagagcctttaaggcctgggccgtcgctagactgagccagagatttcccaaggccgagtttgccgaggtgtccaagctggttaccgacctgaccaaggtgcacacagagtgctgtcacggcgatctgctggaatgcgccgacgatagagccgatctggccaagtacatctgcgagaatcaggacagcatcagctccaagctgaaagaatgctgcgagaagcccctgctcgaaaagagccactgtatcgctgaggtggaaaacgacgagatgcccgccgatctgccttctctggccgccgattttgtggaaagcaaggacgtgtgcaagaattacgccgaggccaaggatgtgttcctgggcatgtttctgtatgagtacgcccgcagacaccccgactactctgttgtgctgctgctgagactggccaaaacctacgagactaccctggaaaagtgctgtgccgccgctgatcctcacgagtgttacgccaaagtgttcgacgagttcaagccactggtggaagaaccccagaacctgatcaaacagaactgcgaactgttcgagcagctgggcgagtacaagttccagaacgccctgctcgtgcggtacaccaagaaggtgccccaggtttcaacccctacactggttgaggtgtcccggaacctgggcaaagtgggcagcaagtgttgcaagcaccctgaggccaagagaatgccctgcgccgaggattacctgagcgtcgtgctgaatcagctgtgcgtgctgcacgagaaaacccctgtgtccgacagagtgaccaagtgctgtaccgagagcctggtcaacagacggccttgctttagcgcccttgaggtggacgagacatacgtgcccaaagagttcaacgccgagacattcaccttccacgccgacatctgtaccctgagcgagaaagagcggcagatcaagaaacagaccgctctggtggaactggtcaagcacaagcccaaggccaccaaagaacaactgaaggccgtgatggacgacttcgccgcctttgtcgagaagtgctgcaaggccgatgacaaagagacatgcttcgccgaagagggaaagaaactggtggccgcctctcaagccgctctgggactttaa

SEQ ID NO: 17 - protein, 1064 aa, Homo sapiens
  FIX_albumin_fusion (1)..(1064): amino acid sequence FIX_albumin fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLTPVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 18 - protein, 1064 aa, Homo sapiens
  FIX_padua_albumin (1)..(1064): amino acid sequence FIX_padua albumin fusion
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLLSTKFTIYNNMFCAGFHEGGRDSCQGDSGGPHVTEVEGTSFLTGIISWGEECAMKGKYGIYTKVSRYVNWIKEKTKLTPVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - DNA, 1983 bp, Homo sapiens
  S/MAR_long (1)..(1983): S/MAR_long derived from IFN-beta
gatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacaacagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatttgatag

SEQ ID NO: 20 - DNA, 742 bp, Homo sapiens
  S/MAR_short (1)..(742): S/MAR_short derived from IFN_beta
aattgagatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaatttgaatgatttaggaagtattccctctgcttctgtcttctgaaagagattgtagaaagttgatacaatttttttttctttaaatatttgatagaatt

SEQ ID NO: 21 - DNA, 587 bp, Encephalomyocarditis virus
  IRES2 (1)..(587): IRES2 sequence
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaac

SEQ ID NO: 22 - DNA, 54 bp, Thosea asigna virus
  T2A (1)..(54): T2A self-cleaving peptide
gagggccgcggaagtcttctaacatgcggtgacgtggaggagaatcccggaccg